(19) United States
(12) Patent Application Publication
(10) Pub. No.: US 2009/0069319 A1
(43) Pub. Date: Mar. 12, 2009

(54) IMIDAZOPYRIDINE ANALOGS AND THEIR USE AS AGONISTS OF THE WNT-BETA-CATENIN CELLULAR MESSAGING SYSTEM

(75) Inventors: Joseph Theodore Lundquist, IV, Limerick, PA (US); Stephen Marc Bowen, Mohnton, PA (US); John Francis Mehlmann, King of Prussia, PA (US); Jeffrey Claude Pelletier, Lafayette Hill, PA (US); Matthew Douglas Vera, Collegeville, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/198,354

(22) Filed: Aug. 26, 2008

Related U.S. Application Data
(60) Provisional application No. 60/966,330, filed on Aug. 27, 2007.

(51) Int. Cl. A61K 31/437; C07D 471/04; A61K 31/5377; A61P 35/00; A61P 19/08; A61P 25/28; C07D 413/14

(52) U.S. Cl. 514/234.2; 546/118; 544/127; 514/303

(57) ABSTRACT

The present invention relates to imidazopyridine analogs, methods of making imidazopyridine analogs, compositions comprising an imidazopyridine analog, and methods for treating canonical Wnt-β-catenin cellular messaging system-related disorders comprising administering to a subject in need thereof an effective amount of an imidazopyridine analog.

### CROSS REFERENCES TO RELATED APPLICATIONS

This application claims priority benefit of U.S. provisional patent application No. 60/966,330, filed Aug. 27, 2007, the entire disclosure of which is hereby incorporated by reference.

### FIELD OF THE INVENTION

The invention relates to imidazopyridine analogs, compositions comprising an imidazopyridine analog, and methods for treating or preventing disorder involving the canonical Wnt-β-catenin cellular messaging system comprising the administration of an effective amount of an imidazopyridine analog.

### BACKGROUND OF THE INVENTION

The Wnt-β-catenin cellular messaging system is essential in many biological processes. It regulates the fate of as-yet undeveloped cells in embryo form. The signals in the Wnt-β-catenin cellular messaging system also direct the development of stem cells in adult organisms (e.g. skin cell, bone cell, liver cell, etc.). At the cellular level, the canonical Wnt-β-catenin cellular messaging system regulates morphology, proliferation, motility and cell fate. The Wnt-β-catenin messaging system has a central role in tumorigenesis and inappropriate activation of this system is observed in several human cancers.

Wnt-β-catenin was first described in humans as a protein which interacts with the cytoplasmic domain of E-cadherin and with Wnt-β-catenin, anchoring the cadherin complex to the actin cytoskeleton. Then, an additional role for mammalian Wnt-β-catenin was discovered; namely, as the key mediator of Wnt-β-catenin messaging.

Chronic activation of the Wnt-β-catenin cellular messaging system has been implicated in the development of a variety of human malignancies, including colorectal carcinomas, hepatocellular carcinomas (HCCs), melanomas, and uterine and ovarian carcinomas.

The Wnt-β-catenin cellular messaging system also plays a role in degenerative disorders such as Alzheimer's disease (AD) and bone disorders.

AD is the most common age-related neurodegenerative disorder. A massive accumulation of beta-amyloid (Abeta) peptide aggregates is likely the pivotal event in AD. Abeta-induced toxicity is accompanied by a varied combination of events including oxidative stress. The Wnt-β-catenin pathway has multiple actions in the cascade of events triggered by Abeta, and drugs with Wnt-β-catenin activity can be therapeutics for AD treatment.

Various bone disorders are also associated with defects in the Wnt-β-catenin messaging system. Signaling through the Wnt-β-catenin pathway increases bone mass through a number of mechanisms, including renewal of stem cells, stimulation of preosteoblast replication, induction of osteoblastogenesis, and inhibition of osteoblast and osteocyte apoptosis.

As discussed above, agonists of the Wnt-β-catenin messaging system are expected to be medicaments useful against cell proliferation disorders, bone disorders, and Alzheimer's disease. Thus, it would be advantageous to have novel agonists of the Wnt-β-catenin messaging system as potential treatment regimens for Wnt-β-catenin messaging system-related disorders. The instant invention is directed to these and other important ends.

### SUMMARY OF THE INVENTION

In one aspect, the invention provides a compound of the Formula (A):

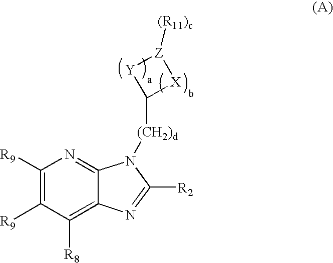

or a tautomer thereof or pharmaceutically acceptable salt thereof,

### or Formula (B)

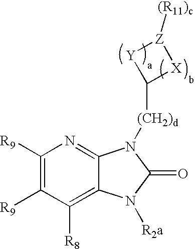

or a tautomer thereof or pharmaceutically acceptable salt thereof,
wherein
each R₁ is independently H, C₁-C₆ alkyl, C₂-C₆ alkenyl, C₂-C₆ alkynyl, C₁-C₆ alkyl carbonyl, C₃-C₈ cycloalkyl, fluorinated C₁-C₆ alkyl, CN, NO₂, halogen, COOR₃, OR₃, S(O)ₙR₃, NHC(O)C₁-C₆ alkyl, N(R₄)(R₅), SO₂N(R₄)(R₅), aryl, heteroaryl, or 3- to 7-membered heterocyclyl, wherein C₁-C₆ alkyl, C₂-C₆ alkenyl, C₂-C₆ alkynyl, C₁-C₆ alkoxyl, aryl, heteroaryl, or 3- to 7-membered heterocyclyl are optionally substituted with one or more R₉ groups;
R₂ is H; C₁-C₆ alkyl; C₃-C₈ cycloalkyl; C₂-C₆ alkenyl, C₂-C₆ alkynyl, CN, NO₂, CO—C₁-C₆ alkyl, halogen, COOR₃, OR₃, S(O)ₙR₃, NHC(O)C₁-C₆ alkyl, N(R₄)(R₅), SO₂N(R₄)(R₅), SH, aryl optionally fused to a heterocyclic ring, heteroaryl, 3- to 7-membered heterocyclyl, or fused aryl heterocyclyl, wherein C₁-C₆ alkyl, C₂-C₆ alkenyl, C₂-C₆ alkynyl, aryl, heteroaryl, or 3- to 7-membered heterocyclyl, fused arylheterocycle, or OR₃ are optionally substituted with one or more R₁ groups;
R₂ₐ is C₁-C₆ alkyl or C₃-C₈ cycloalkyl, wherein each R₂ₐ is optionally substituted with halogen, hydroxyl, or C₁-C₆ alkoxyl;
R₄ and R₅ are each independently H, aryl, heteroaryl, C₁-C₆ alkyl, C₃-C₈ cycloalkyl, 3- to 7-membered heterocyclyl, aryl-C₁-C₆ alkyl, heteroaryl-C₁-C₆ alkyl, C₃-C₈ cycloalkyl-C₁-C₆ alkyl, 3- to 7-membered-heterocyclyl-C₁-C₆ alkyl, 3- to 7-membered-heterocyclyl-C₁-C₆alkyl-aryl, 3- to 7-membered-heterocyclyl-alkoxyaryl, 3- to 7-membered heterocyclyl-C₁-C₆ alkyl-heteroaryl, 3- to 7-membered-heterocyclyl-C₁-C₆ alkoxy-heteroaryl, C₁-C₆ alkoxyaryl, C₁-C₆ alkylamine-aryl, 3- to 7-membered heterocyclyl-C₁-C₆ alkylamine-aryl, 3- to 7-membered heterocyclyl-C₁-C₆ alkylamine-heteroaryl; wherein all except H are optionally substituted with C₁-C₆ alkyl optionally substituted with halogen, hydroxyl, or C₁-C₆ alkoxyl; or R₄ and R₅ when taken together with the nitrogen to which they are attached form a 3- to 8-membered heterocyclyl having 0, 1, or 2 additional heteroatoms and optionally substituted with C₁-C₆ alkyl optionally substituted with halogen, hydroxyl, or C₁-C₆ alkoxyl;
R₆ and R₇ are each independently H, NH₂, CN, NO₂, C₁-C₆ alkyl, C₂-C₆ alkenyl, C₂-C₆ alkynyl, COOH, COOR₃, halogen, C₁-C₆ alkoxy, C₃-C₈ cycloalkyl, CF₃, S(O)ₙR₃ or OR₃; or R₆ and R₇ when taken together with the ring to which they are attached form C₅-C₇ cycloalkyl or C₆ aryl, optionally substituted with one or more R₁ groups;
R₈ is H or naphthyl optionally substituted with one or more R₁ groups;
each R₉ is independently H or C₆-C₁₀ aryl;
each R₁₁ is independently H, C₁-C₆ alkyl, C₂-C₆ alkenyl, C₂-C₆ alkynyl, C₁-C₆ alkyl carbonyl, C₃-C₈ cycloalkyl, fluorinated C₁-C₆ alkyl, CF₃, CN, NO₂, NH₂, halogen, COOR₃, OR₃, S(O)ₙR₃, NHC(O)C₁-C₆ alkyl, N(R₄)(R₅), SO₂N(R₄)(R₅), C(O)NR4R5, aryl, heteroaryl, and 3- to 7-membered heterocyclyl, wherein aryl, heteroaryl, heterocyclyl, C₁-C₆ alkyl, C₂-C₆ alkenyl, C₂-C₆ alkynyl, C₁-C₆ carbonyl, or cycloalkyl are optionally substituted with aryl, C₁-C₆ alkyl optionally substituted with halogen; halogen; hydroxyl; or C₁-C₆ alkoxy;
X is CR₆R₆ or CHR₆CHR₇;
Y is CR₁R₇;
Z is CH, N or O, with the proviso that when Z is O, c is zero;
a is 1, 2, 3 or 4;
b is 0, 1 or 2;
c is 0, 1 or 2;
d is 0, 1, 2 or 3; and
n is 0, 1 or 2.

In one aspect, the invention provides a compound of Formula (A₀):

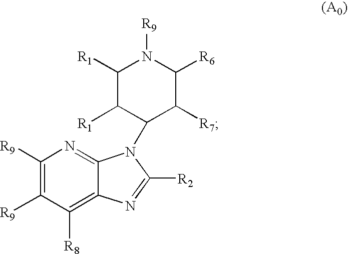

or a tautomer thereof or pharmaceutically acceptable salt thereof,

each R₁ is independently selected from H, C₁-C₆ alkyl, C₂-C₆ alkenyl, C₂-C₆ alkynyl, ═O, C₁-C₆ carbonyl, cycloalkyl, fluorinatedalkyl, CF₃, CN, NO₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, or heterocyclyl, wherein C₁-C₆ alkyl, C₂-C₆ alkenyl, C₂-C₆ alkynyl, C₁-C₆ alkoxy, aryl, heteroaryl, or heterocyclyl, are optionally substituted with one or more substituents selected from H, C₁-C₆ alkyl, C₂-C₆ alkenyl, C₂-C₆ alkynyl, ═O, C₁-C₆ carbonyl, cycloalkyl, fluorinatedalkyl, CF₃, CN, NO₂, halogen, COOR₃, C₁-C₆ alkoxy, OR₃, S(O)ₙ R₃, NHC(O)C₁-C₆ alkyl, N(R₄)(R₅), SO₂N(R₄)(R₅), aryl, heteroaryl, or heterocyclyl, wherein C₁-C₆ alkyl, C₂-C₆ alkenyl, C₂-C₆ alkynyl, C₁-C₆ alkoxy, aryl, heteroaryl, and heterocyclyl;

R₂ is selected from H, C₁-C₆ alkyl, C₂-C₆ alkenyl, C₂-C₆ alkynyl, C₁-C₆ carbonyl, cycloalkyl, fluorinatedalkyl, CF₃, CN, NO₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, heterocyclyl, or fused aryl-heterocyclyl, wherein C₁-C₆ alkyl, C₂-C₆ alkenyl, C₂-C₆ alkynyl, C₁-C₆ alkoxy, aryl, heteroaryl, heterocyclyl, or fused aryl-heterocyclyl are optionally substituted with one or more R₁ groups;

R₃ is H, CF₃, C₁-C₆ alkyl, C₃-C₇ cycloalkyl, heterocyclyl, heterocyclyl alkyl, aryl, arylalkyl, heteroaryl, whereas all except H and CF₃ are optionally substituted with zero, one or more R₁ groups;

R₄ and R₅ are each independently H, aryl, heteroaryl, C₁-C₈ alkyl, cycloalkyl, heterocyclyl, arylalkyl, heteroarylalkyl, cycloalkyl-alkyl, heterocyclyl-alkyl, heterocyclyl-alkyl-aryl, heterocyclyl-alkoxyaryl, heterocyclyl-alkyl-heteroaryl, heterocyclyl-alkoxy-heteroaryl, alkoxyaryl, alkylamine-aryl, heterocyclyl-alkylamine-aryl, heterocyclyl-alkylamine-heteroaryl, whereas all except H are optionally substituted with zero, one or more R₁ groups; or R₄ and R₅ when taken together with the nitrogen to which they are attached form a 4 to 8 membered ring with 0, 1, or 2 additional heteroatoms optionally substituted with one or more R₁ groups;

R₆ and R₇ are each independently H, NH₂, CN, NO₂, C₁-C₆ alkyl, C₂-C₆ alkenyl, C₂-C₆ alkynyl, COOH, COOR₃, halogen, C₁-C₆ alkoxy, cycloalkyl, CF₃, S(O)ₙR₃ or OR₃; or R₆ and R₇ when taken together with the ring to which they are attached form C₅-C₇ cycloalkyl or C₆ aryl, optionally substituted with one or more R₁ groups;

R₈ is naphthlyl optionally substituted with one or more R₁ groups;

each R₉ is independently selected from H, C₁-C₆ alkyl, C₂-C₆ alkenyl, C₂-C₆ alkynyl, C₁-C₆ carbonyl, cycloalkyl, fluorinatedalkyl, CF₃, CN, NO₂, NH₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, heterocyclyl, wherein aryl, heteroaryl, heterocyclyl, C₁-C₆ alkyl, C₂-C₆ alkenyl, C₂-C₆ alkynyl, C₁-C₆ carbonyl, fluorinatedalkyl, or cycloalkyl are optionally substituted with one or more R₁ groups; and

n is 0, 1, or 2.

In another aspect the invention provides compounds of the formula (A₁):

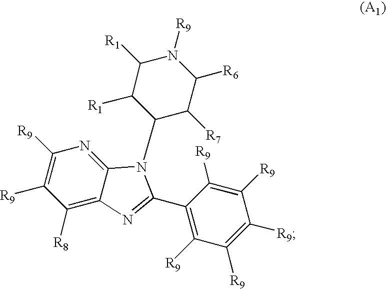

or tautomers thereof or pharmaceutically acceptable salts thereof, wherein R₁, R₆, R₇, R₈ and R₉ are defined as in Formula (A₀).

In other aspects, the invention provides pharmaceutical compositions comprising compounds or tautomers thereof or pharmaceutically acceptable salts of compounds of Formula A₀, and Formula A₁, and a pharmaceutically acceptable carrier.

In one aspect, the compounds or tautomers thereof or pharmaceutically acceptable salts of the compounds of Formula A₀ and Formula A₁ are useful as canonical Wnt-β-catenin cellular messaging system agonists.

In some embodiments, the invention provides methods for treating a canonical Wnt-β-catenin cellular messaging system related disorder, comprising administering to a mammal in need thereof a compound or a tautomer thereof or pharmaceutically acceptable salt of a compound of Formula A₀, and Formula A₁ in an amount effective to treat a canonical Wnt-β-catenin cellular messaging system related disorder.

### DETAILED DESCRIPTION OF THE INVENTION

The following definitions are used in connection with the imidazopyridine analogs of the present invention:

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 ring carbons. Examples of an aryl group include, but are not limited to, phenyl, naphthyl, anthracenyl, tetracenyl, and phenanthrenyl. An aryl group can be unsubstituted or substituted with one or more of the following groups: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Heteroaryl” refers to mono and bicyclic aromatic groups of 4 to 10 ring atoms containing at least one heteroatom, eg 1 to 4 heteroatoms, the same or different. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Arylalkyl” refers to an aryl group with at least one alkyl substitution. Examples of arylalkyl include, but are not limited to, benzyl, toluenyl, phenylethyl, xylenyl, phenylbutyl, phenylpentyl, and ethylnaphthyl. An arylalkyl group can be unsubstituted or substituted with one or more of the following groups: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Heteroarylalkyl” refers to a heteroaryl group with at least one alkyl substitution. A heteroarylalkyl group can be unsubstituted or substituted with one or more of the following groups: H, halogen, CN, OH, aryl, arylalkyl, heteroaryl, heteroarylalkyl, C₁₋₆ alkyl, C₂₋₆ alkenyl, C₂₋₆ alkynyl, C₁₋₃ fluorinatedalkyl, C₃₋₆ cycloalkyl, C₃₋₆cycloalkyl-C₁₋₃alkyl, NO₂, NH₂, NHC₁₋₆ alkyl, N(C₁₋₆ alkyl)₂, NHC₃₋₆ cycloalkyl, N(C₃₋₆ cycloalkyl)₂, NHC(O)C₁₋₆ alkyl, NHC(O)C₃₋₆ cycloalkyl, NHC(O)NHC₁₋₆ alkyl, NHC(O)NHC₃₋₆ cycloalkyl, SO₂NH₂, SO₂NHC₁₋₆ alkyl, SO₂NHC₃₋₆ cycloalkyl SO₂N(C₁₋₆ alkyl)₂, SO₂N(C₃₋₆ cycloalkyl)₂, NHSO₂C₁₋₆ alkyl, NHSO₂C₃₋₆ cycloalkyl, CO₂C₁₋₆ alkyl, CO₂C₃₋₆ cycloalkyl, CONHC₁₋₆ alkyl, CONHC₃₋₆ cycloalkyl, CON(C₁₋₆ alkyl)₂, CON(C₃₋₆ cycloalkyl)₂OH, OC₁₋₃ alkyl, C₁₋₃ fluorinatedalkyl, OC₃₋₆ cycloalkyl, OC₃₋₆ cycloalkyl-C₁₋₃ alkyl, SH, SOₓC₁₋₃ alkyl, C₃₋₆ cycloalkyl, or SOₓC₃₋₆ cycloalkyl-C₁₋₃ alkyl, where x is 0, 1, or 2.

“Alkylamine” refers to an alkyl group attached to the nitrogen of an amine. Examples of alkylamines include, but are not limited to, methylamine, ethylamine, propylamine, butylamine, diethylamine, and isobutylamine.

“Alkoxy” refers to an alkyl group linked to oxygen atom that is further linked to a carbon chain or ring. Examples of an alkoxy include, but are not limited to, methoxy, ethoxy, phenoxy, butoxy, isopropoxy, and butoxy.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, isopropyl, n-pentyl, isopentyl, neopentyl, and hexyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₃-C₆ alkenyl” refers to a straight or branched chain unsaturated hydrocarbon containing 3-6 carbon atoms and at least one double bond. Examples of a C₃-C₆ alkenyl group include, but are not limited to, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆-alkoxy include, but are not limited to, methoxy, ethoxy, isopropoxy, butoxy, n-pentoxy, isopentoxy, neopentoxy, and hexoxy.

“C₃-C₆ cycloalkyl” referres to a cyclic saturated hydrocarbon containing 3-6 carbon atoms. Examples of a C₃-C₆ cycloalkyl group include, but are not limited to, cyclopropane, cyclobutane, cyclopentane, and cyclohexane.

“C₃-C₆ cycloalkyl-C₁-C₃ alkyl” refers to a cyclic saturated hydrocarbon containing 3-6 carbon atoms that is further substituted with a straight or branched chain hydrocarbon containing 1-3 carbon atoms. Examples of a C₃-C₆ cycloalkyl-C₁-C₃ alkyl group include, but are not limited to, propylcyclopropane, propylcyclobutane, ethylcyclopropane, propylcyclopentane, and methylcyclohexane.

The term “3- to 7-membered heterocycle” refers to: (i) a 3- to 7-membered non-aromatic monocyclic cycloalkyl in which 1 of the ring carbon atoms has been replaced with an N, O or S atom; or (ii) a 5-, 6-, or 7-membered aromatic or non-aromatic monocyclic cycloalkyl in which 1-4 of the ring carbon atoms have been independently replaced with a N, O or S atom. The non-aromatic 3- to 7-membered monocyclic heterocycles can be attached via a ring nitrogen, sulfur, or carbon atom. The aromatic 3- to 7-membered monocyclic heterocycles are attached via a ring carbon atom. Representative examples of a 3- to 7-membered monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, triazolyl, In one embodiment, the 3- to 7-membered monocyclic heterocycle group is substituted with one or more of the following groups: -halo, —O—(C₁-C₆ alkyl), —OH, —CN, —COOR′, —OC(O)R′, aryl, alkylaryl, —N(R′)₂, —NHC(O)R′, —C(O)NHR′, —NHC(O)OR′, NH(SO₂R′), or NH(SO₂N(R′)₂) groups wherein each R′ is independently —H or unsubstituted —C₁-C₆ alkyl. In another embodiment, one or more of the ring nitrogens is substituted with R′, C(O)R′, C(O)H, C(NH)N(R′), C(O)OR′, C(O)N(R′), SO₂R′, heteroaryl, C(O)CF₃.

“C₁-C₃ fluorinated alkyl” refers to an saturated straight or branched chain hydrocarbon containing 1-3 carbon atoms that can be further substituted with other functional groups, eg., ═O. Examples of a C₁-C₃ fluorinated alkyl are trifluoromethyl, 1,1,1-trifluoroethyl, and trifluoroacetyl.

“Fused aryl-heterocyclyl” refers to an aryl ring system fused at least two positions to a heterocyclyl ring systems. Examples of a fused aryl-heterocyclyl include, but are not limited to, benzodioxinyl, and benzodioxolyl.

“Amino protecting group” refers to t-butyloxycarbonyl amino protecting group

A “subject” is a mammal, e.g., a human, mouse, rat, guinea pig, dog, cat, horse, cow, pig, or non-human primate, such as a monkey, chimpanzee, baboon or rhesus.

The invention also includes pharmaceutical compositions comprising an effective amount of an imidazopyridine analog and a pharmaceutically acceptable carrier. The invention includes an imidazopyridine analog when provided as a tautomer thereof or pharmaceutically acceptable salt, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection an imidazopyridine analog is an amount effective for treating or preventing a disorder associated with the canonical Wnt-β-catenin cellular messaging system.

As used herein, the recitations “zero, one or more” and “one or more” mean that the upper limit of the number of substituents in a given group is determined by the maximum number of hydrogens in that group.

The following abbreviations are used herein and have the indicated definitions: ACN is acetonitrile, DCM is dichloromethane, DMSO is dimethylsulfoxide, FBS is fetal bovine serum, HPLC is high pressure liquid chromatography, MeOH is methanol, MS is mass spectrometry, NMP is N-methyl-2-pyrrolidone, RPMI is Roswell Park Memorial Institute, TFA is trifluoroacetic acid, and VLUX is a device for measuring luminescence.

### The Imidazopyridine Analogs of Formula (A)

In one aspect, the invention provides a compound of the Formula (A):

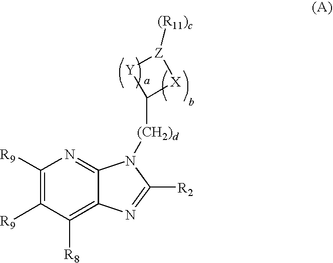

or a tautomer thereof or pharmaceutically acceptable salt thereof, wherein X, Y, Z, a, b, c, d, R₁, R₂, R₈, R₉ and R₁₁ are as defined above.

In one embodiment, Z is N.

In one embodiment, a is 1, 2 or 3.

In one embodiment, b is 1 or 2

In one embodiment, d is 0.

In one embodiment, d is 1.

In one embodiment, R₁, R₆ and R₇ are each independently H or C₁-C₆ alkyl, R₁ being optionally substituted as defined above.

In one embodiment, each R₉ is independently H or naphthyl (eg naphth-2-yl) such as 5-(naphth-2-yl) or 6-(naphth-2-yl).

In one embodiment, R₈ is H or naphthyl, e.g., naphth-2-yl.

In one embodiment, ring containing X and Y is piperidinyl, pyrrolidinyl or azetidinyl, each being optionally independently substituted as defined above.

In one embodiment, R₂ is H; C₁-C₆ alkyl; C₃-C₈ cycloalkyl; 5- to 10-membered hetroaryl optionally substituted with one, two or three substituents independently selected from halogen, CN, OH, OC₁-C₆ alkyl optionally substituted with halogen, OC₆-C₁₀ aryl, C₁-C₆ alkyl optionally substituted with halogen, C₂-C₆ alkenyl, NO2, heterocyclyl, NHC(O)C₁-C₆ alkyl, NR₄R₅; C₆-C₁₀ aryl or CO—C₁-C₆ alkyl; heterocyclyl optionally substituted with one, two or three COOC₁-C₆ alkyl; C₆-C₁₀ aryl optionally fused to a heterocyclic ring or optionally substituted with one, two or three substituents independently selected from halogen, CN, OH, OC₁-C₆ alkyl optionally substituted with halogen, OC₆-C₁₀ aryl, C₁-C₆ alkyl optionally substituted with halogen, C₂-C₆ alkenyl, NO₂, heterocyclyl, NHC(O)C₁-C₆ alkyl, NR₄R₅; OH; or SH.

In one embodiment, R₂ is methyl, ethyl, propyl, i-propyl, butyl, s-butyl, t-butyl or i-butyl.

In one embodiment, R₂ is cyclohexyl.

In one embodiment, R₂ is pyridyl, n-oxo pyridyl, pyrrolyl, indolyl, imidazolyl, thiophenyl, benzothiophenyl, quinolinyl, furanyl, or benzofuranyl, each being optionally independently substituted as defined above.

In one embodiment, R₂ is piperidinyl or pyrimidinedion-yl, each being optionally independently substituted as defined.

In one embodiment, R₂ is phenyl or naphthyl, each being optionally independently substituted as defined above.

In one embodiment, R₁₁ is H; COOR₃; C₁-C₆ alkyl carbonyl; CONR₄R₅; SO₂NR₄R₅; SO₂R₃; C₁-C₆ alkyl optionally substituted with one or two substituents independently selected from the group consisting of aryl and halogen; or NR₄R₅.

In one embodiment, R₁₁ is COO—C₁-C₆ alkyl.

In one embodiment, R₁₁ is SO₂aryl or SO₂C₁-C₆ alkyl.

In one aspect, the invention provides compounds of the Formula (A₂):

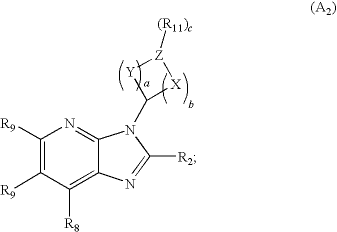

or tautomers thereof or pharmaceutically acceptable salts thereof, wherein R₂, R₈, R₉, R₁₁, X, Y, a, b, c and d are defined as above for Formula (A).
Illustrative compounds of Formula A are exemplified by the compounds in the following table:

Compound

7-(2-naphthyl)-3-[1-(trifluoroacetyl)piperidin-4-yl]-3H-imidazo[4,5-b]pyridine
methyl 4-[7-(2-naphthyl)-3H-imidazo[4,5-b]pyridin-3-yl]piperidine-1-carboxylate
N,N-dimethyl-4-[7-(2-naphthyl)-3H-imidazo[4,5-b]pyridin-3-yl]piperidine-1-carboxamide
N-ethyl-4-[7-(2-naphthyl)-3H-imidazo[4,5-b]pyridin-3-yl]piperidine-1-carboxamide
N-isopropyl-4-[7-(2-naphthyl)-3H-imidazo[4,5-b]pyridin-3-yl]piperidine-1-carboxamide
3-(1-benzylpiperidin-4-yl)-7-(2-naphthyl)-3H-imidazo[4,5-b]pyridine
3-[1-(methylsulfonyl)piperidin-4-yl]-7-(2-naphthyl)-3H-imidazo[4,5-b]pyridine
N-cyclohexyl-4-[7-(2-naphthyl)-3H-imidazo[4,5-b]pyridin-3-yl]piperidine-1-carboxamide
3-[1-(morpholin-4-ylcarbonyl)piperidin-4-yl]-7-(2-naphthyl)-3H-imidazo[4,5-b]pyridine
N-(3′,6′-dihydroxy-3-oxo-3H-spiro[2-benzofuran-1,9′-xanthen]-5-yl)-4-[7-(2-naphthyl)-3H-imidazo[4,5-
b]pyridin-3-yl]piperidine-1-carbothioamide
N,N-dimethyl-4-[7-(2-naphthyl)-3H-imidazo[4,5-b]pyridin-3-yl]piperidine-1-sulfonamide
7-(2-naphthyl)-3-(piperidin-4-ylmethyl)-3H-imidazo[4,5-b]pyridine
3-[3-(1H-imidazol-1-yl)propyl]-7-(2-naphthyl)-3H-imidazo[4,5-b]pyridine
2-methyl-7-(2-naphthyl)-3-[(3R)-pyrrolidin-3-yl]-3H-imidazo[4,5-b]pyridine
2-methyl-3-[2-(4-methylphenyl)ethyl]-7-(2-naphthyl)-3H-imidazo[4,5-b]pyridine
7-(2-naphthyl)-3-[(3S)-piperidin-3-yl]-3H-imidazo[4,5-b]pyridine
3-[2-(4-methylphenyl)ethyl]-7-(2-naphthyl)-3H-imidazo[4,5-b]pyridine
2-methyl-7-(2-naphthyl)-3-(piperidin-4-ylmethyl)-3H-imidazo[4,5-b]pyridine
3-azetidin-3-yl-7-(2-naphthyl)-3H-imidazo[4,5-b]pyridine
7-(2-naphthyl)-3-[(3R)-pyrrolidin-3-yl]-3H-imidazo[4,5-b]pyridine
7-(2-naphthyl)-3-[(3S)-pyrrolidin-3-yl]-3H-imidazo[4,5-b]pyridine
N-isopropyl-4-[7-(2-naphthyl)-2-oxo-1,2-dihydro-3H-imidazo[4,5-b]pyridin-3-yl]piperidine-1-
carboxamide
3-(1-acetylpiperidin-4-yl)-1-methyl-7-(2-naphthyl)-1,3-dihydro-2H-imidazo[4,5-b]pyridin-2-one
N,N-dimethyl-4-[1-methyl-7-(2-naphthyl)-2-oxo-1,2-dihydro-3H-imidazo[4,5-b]pyridin-3-yl]piperidine-
1-carboxamide
2-methyl-7-(2-naphthyl)-3-[(3S)-pyrrolidin-3-yl]-3H-imidazo[4,5-b]pyridine
2-methyl-7-(2-naphthyl)-3-[(3R)-piperidin-3-yl]-3H-imidazo[4,5-b]pyridine
3-[1-(morpholin-4-ylcarbonyl)piperidin-4-yl]-7-(2-naphthyl)-1,3-dihydro-2H-imidazo[4,5-b]pyridin-2-
one
7-(2-naphthyl)-3-[(3R)-piperidin-3-yl]-3H-imidazo[4,5-b]pyridine
3-azetidin-3-yl-2-methyl-7-(2-naphthyl)-3H-imidazo[4,5-b]pyridine
2-methyl-7-(2-naphthyl)-3-[(3S)-piperidin-3-yl]-3H-imidazo[4,5-b]pyridine
N-isopropyl-4-[1-methyl-7-(2-naphthyl)-2-oxo-1,2-dihydro-3H-imidazo[4,5-b]pyridin-3-yl]piperidine-
1-carboxamide
6-(2-naphthyl)-3-piperidin-4-yl-2-(1H-pyrrol-2-yl)-3H-imidazo[4,5-b]pyridine
2-(1-methyl-1H-indol-2-yl)-6-(2-naphthyl)-3-piperidin-4-yl-3H-imidazo[4,5-b]pyridine
2-(1H-indol-3-yl)-6-(2-naphthyl)-3-piperidin-4-yl-3H-imidazo[4,5-b]pyridine
6-(2-naphthyl)-3-piperidin-4-yl-3H-imidazo[4,5-b]pyridine
2-methyl-6-(2-naphthyl)-3-piperidin-4-yl-3H-imidazo[4,5-b]pyridine
4-[6-(2-naphthyl)-3-piperidin-4-yl-3H-imidazo[4,5-b]pyridin-2-yl]quinoline
2-(4-isopropylphenyl)-6-(2-naphthyl)-3-piperidin-4-yl-3H-imidazo[4,5-b]pyridine
2-(2,4-dichlorophenyl)-6-(2-naphthyl)-3-piperidin-4-yl-3H-imidazo[4,5-b]pyridine
2-(3-chlorophenyl)-6-(2-naphthyl)-3-piperidin-4-yl-3H-imidazo[4,5-b]pyridine
6-(2-naphthyl)-3-piperidin-4-yl-2-pyridin-4-yl-3H-imidazo[4,5-b]pyridine
6-(2-naphthyl)-3-piperidin-4-yl-2-(2-thienyl)-3H-imidazo[4,5-b]pyridine
2-(2-methoxypyridin-3-yl)-6-(2-naphthyl)-3-piperidin-4-yl-3H-imidazo[4,5-b]pyridine
6-(2-naphthyl)-2-(3-nitrophenyl)-3-piperidin-4-yl-3H-imidazo[4,5-b]pyridine
2-ethyl-6-(2-naphthyl)-3-piperidin-4-yl-3H-imidazo[4,5-b]pyridine
1-methyl-3-[1-(morpholin-4-ylcarbonyl)piperidin-4-yl]-7-(2-naphthyl)-1,3-dihydro-2H-imidazo[4,5-
b]pyridin-2-one
1-methyl-3-[1-(methylsulfonyl)piperidin-4-yl]-7-(2-naphthyl)-1,3-dihydro-2H-imidazo[4,5-b]pyridin-2-
one
6-(2-naphthyl)-3-piperidin-4-yl-2-pyridin-2-yl-3H-imidazo[4,5-b]pyridine
2-(3-methoxyphenyl)-6-(2-naphthyl)-3-piperidin-4-yl-3H-imidazo[4,5-b]pyridine
2-(4-methylphenyl)-6-(2-naphthyl)-3-piperidin-4-yl-3H-imidazo[4,5-b]pyridine
4-[6-(2-naphthyl)-3-piperidin-4-yl-3H-imidazo[4,5-b]pyridin-2-yl]phenol
2-butyl-6-(2-naphthyl)-3-piperidin-4-yl-3H-imidazo[4,5-b]pyridine
2-(2-fluorophenyl)-6-(2-naphthyl)-3-piperidin-4-yl-3H-imidazo[4,5-b]pyridine
2-isopropyl-6-(2-naphthyl)-3-piperidin-4-yl-3H-imidazo[4,5-b]pyridine
2-(2-furyl)-6-(2-naphthyl)-3-piperidin-4-yl-3H-imidazo[4,5-b]pyridine
2-(1-benzothien-2-yl)-6-(2-naphthyl)-3-piperidin-4-yl-3H-imidazo[4,5-b]pyridine
2-cyclohexyl-6-(2-naphthyl)-3-piperidin-4-yl-3H-imidazo[4,5-b]pyridine
6-(2-naphthyl)-3-piperidin-4-yl-2-propyl-3H-imidazo[4,5-b]pyridine
2-(2-methoxyphenyl)-6-(2-naphthyl)-3-piperidin-4-yl-3H-imidazo[4,5-b]pyridine
2-(2,4-difluorophenyl)-6-(2-naphthyl)-3-piperidin-4-yl-3H-imidazo[4,5-b]pyridine
2-(1-benzofuran-5-yl)-6-(2-naphthyl)-3-piperidin-4-yl-3H-imidazo[4,5-b]pyridine
2-(5-methyl-2-furyl)-6-(2-naphthyl)-3-piperidin-4-yl-3H-imidazo[4,5-b]pyridine
2-(1-benzofuran-2-yl)-6-(2-naphthyl)-3-piperidin-4-yl-3H-imidazo[4,5-b]pyridine
2-(4-tert-butylphenyl)-6-(2-naphthyl)-3-piperidin-4-yl-3H-imidazo[4,5-b]pyridine
2-(2,4-dimethylphenyl)-6-(2-naphthyl)-3-piperidin-4-yl-3H-imidazo[4,5-b]pyridine
2-(1-naphthyl)-6-(2-naphthyl)-3-piperidin-4-yl-3H-imidazo[4,5-b]pyridine
3-[6-(2-naphthyl)-3-piperidin-4-yl-3H-imidazo[4,5-b]pyridin-2-yl]phenol
6-(2-naphthyl)-3-piperidin-4-yl-2-pyridin-3-yl-3H-imidazo[4,5-b]pyridine
2-(4-morpholin-4-yl-3-nitrophenyl)-6-(2-naphthyl)-3-piperidin-4-yl-3H-imidazo[4,5-b]pyridine
8-[6-(2-naphthyl)-3-piperidin-4-yl-3H-imidazo[4,5-b]pyridin-2-yl]quinoline
5-[6-(2-naphthyl)-3-piperidin-4-yl-3H-imidazo[4,5-b]pyridin-2-yl]quinoline
2-(1-methyl-1H-indol-3-yl)-6-(2-naphthyl)-3-piperidin-4-yl-3H-imidazo[4,5-b]pyridine
2-isobutyl-6-(2-naphthyl)-3-piperidin-4-yl-3H-imidazo[4,5-b]pyridine
6-(2-naphthyl)-2-(2-nitrophenyl)-3-piperidin-4-yl-3H-imidazo[4,5-b]pyridine
6-(2-naphthyl)-2-phenyl-3-piperidin-4-yl-3H-imidazo[4,5-b]pyridine
2-(3-methylphenyl)-6-(2-naphthyl)-3-piperidin-4-yl-3H-imidazo[4,5-b]pyridine
2,6-di-2-naphthyl-3-piperidin-4-yl-3H-imidazo[4,5-b]pyridine
tert-butyl (3R)-3-[7-(2-naphthyl)-3H-imidazo[4,5-b]pyridin-3-yl]pyrrolidine-1-carboxylate
tert-butyl (3S)-3-[7-(2-naphthyl)-3H-imidazo[4,5-b]pyridin-3-yl]pyrrolidine-1-carboxylate
6-(2-naphthyl)-3-piperidin-4-yl-2-[2-(trifluoromethyl)phenyl]-3H-imidazo[4,5-b]pyridine
6-(2-naphthyl)-3-piperidin-4-yl-2-[4-(trifluoromethoxy)phenyl]-3H-imidazo[4,5-b]pyridine
4-[6-(2-naphthyl)-3-piperidin-4-yl-3H-imidazo[4,5-b]pyridin-2-yl]benzonitrile
2-(1H-imidazol-2-yl)-6-(2-naphthyl)-3-piperidin-4-yl-3H-imidazo[4,5-b]pyridine
2-(3,5-dichloropyridin-4-yl)-6-(2-naphthyl)-3-piperidin-4-yl-3H-imidazo[4,5-b]pyridine
2-(1,3-benzodioxol-5-yl)-6-(2-naphthyl)-3-piperidin-4-yl-3H-imidazo[4,5-b]pyridine
tert-butyl (3S)-3-[2-methyl-7-(2-naphthyl)-3H-imidazo[4,5-b]pyridin-3-yl]piperidine-1-carboxylate
5-(2-naphthyl)-3-piperidin-4-yl-3H-imidazo[4,5-b]pyridine
4-[6-(2-naphthyl)-3-piperidin-4-yl-3H-imidazo[4,5-b]pyridin-2-yl]-N,N-diphenylaniline
6-(2-naphthyl)-3-piperidin-4-yl-2-[4-(trifluoromethyl)phenyl]-3H-imidazo[4,5-b]pyridine
2-[2-chloro-3-(trifluoromethyl)phenyl]-6-(2-naphthyl)-3-piperidin-4-yl-3H-imidazo[4,5-b]pyridine
6-(2-naphthyl)-2-(4-nitrophenyl)-3-piperidin-4-yl-3H-imidazo[4,5-b]pyridine
2-(2-methyl-1H-imidazol-4-yl)-6-(2-naphthyl)-3-piperidin-4-yl-3H-imidazo[4,5-b]pyridine
6-(2-naphthyl)-3-piperidin-4-yl-2-[3-(trifluoromethoxy)phenyl]-3H-imidazo[4,5-b]pyridine
tert-butyl 3-[7-(2-naphthyl)-3H-imidazo[4,5-b]pyridin-3-yl]azetidine-1-carboxylate
tert-butyl (3R)-3-[7-(2-naphthyl)-3H-imidazo[4,5-b]pyridin-3-yl]piperidine-1-carboxylate
tert-butyl (3R)-3-[2-methyl-7-(2-naphthyl)-3H-imidazo[4,5-b]pyridin-3-yl]piperidine-1-carboxylate
2-(4-methoxyphenyl)-6-(2-naphthyl)-3-piperidin-4-yl-3H-imidazo[4,5-b]pyridine
6-(2-naphthyl)-3-piperidin-4-yl-2-[2-(trifluoromethoxy)phenyl]-3H-imidazo[4,5-b]pyridine
2-(2-chlorophenyl)-6-(2-naphthyl)-3-piperidin-4-yl-3H-imidazo[4,5-b]pyridine
2-(4-methyl-1H-imidazol-5-yl)-6-(2-naphthyl)-3-piperidin-4-yl-3H-imidazo[4,5-b]pyridine
2-(1-acetyl-1H-indol-3-yl)-6-(2-naphthyl)-3-piperidin-4-yl-3H-imidazo[4,5-b]pyridine
2-(2,3-dihydro-1,4-benzodioxin-6-yl)-6-(2-naphthyl)-3-piperidin-4-yl-3H-imidazo[4,5-b]pyridine
2-chloro-3-[6-(2-naphthyl)-3-piperidin-4-yl-3H-imidazo[4,5-b]pyridin-2-yl]quinoline
7-(2-naphthyl)-3-piperidin-4-yl-3H-imidazo[4,5-b]pyridine
2-methyl-7-(2-naphthyl)-3-piperidin-4-yl-3H-imidazo[4,5-b]pyridine
3-[6-(2-naphthyl)-3-piperidin-4-yl-3H-imidazo[4,5-b]pyridin-2-yl]benzene-1,2-diol
3-[6-(2-naphthyl)-3-piperidin-4-yl-3H-imidazo[4,5-b]pyridin-2-yl]-4-nitrophenol
2-(2,6-difluorophenyl)-6-(2-naphthyl)-3-piperidin-4-yl-3H-imidazo[4,5-b]pyridine
2-[4-fluoro-2-(trifluoromethyl)phenyl]-6-(2-naphthyl)-3-piperidin-4-yl-3H-imidazo[4,5-b]pyridine
2-(1-ethyl-5-methyl-1H-imidazol-4-yl)-6-(2-naphthyl)-3-piperidin-4-yl-3H-imidazo[4,5-b]pyridine
2-(5-methoxy-1H-indol-3-yl)-6-(2-naphthyl)-3-piperidin-4-yl-3H-imidazo[4,5-b]pyridine
2-(3-furyl)-6-(2-naphthyl)-3-piperidin-4-yl-3H-imidazo[4,5-b]pyridine
5-[6-(2-naphthyl)-3-piperidin-4-yl-3H-imidazo[4,5-b]pyridin-2-yl]pyrimidine-2,4(1H,3H)-dione
6-(2-naphthyl)-3-piperidin-4-yl-2-[3-(trifluoromethyl)phenyl]-3H-imidazo[4,5-b]pyridine
2-[2,5-bis(trifluoromethyl)phenyl]-6-(2-naphthyl)-3-piperidin-4-yl-3H-imidazo[4,5-b]pyridine
2-(4-fluorophenyl)-6-(2-naphthyl)-3-piperidin-4-yl-3H-imidazo[4,5-b]pyridine
N-{4-[6-(2-naphthyl)-3-piperidin-4-yl-3H-imidazo[4,5-b]pyridin-2-yl]phenyl}acetamide
2-(1-benzothien-3-yl)-6-(2-naphthyl)-3-piperidin-4-yl-3H-imidazo[4,5-b]pyridine
2-(5-methyl-1H-indol-3-yl)-6-(2-naphthyl)-3-piperidin-4-yl-3H-imidazo[4,5-b]pyridine
2-[6-(2-naphthyl)-3-piperidin-4-yl-3H-imidazo[4,5-b]pyridin-2-yl]quinoline
7-(2-naphthyl)-3-piperidin-4-yl-1,3-dihydro-2H-imidazo[4,5-b]pyridin-2-one
7-(2-naphthyl)-3-(2,2,6,6-tetramethylpiperidin-4-yl)-3H-imidazo[4,5-b]pyridine
3-cyclohexyl-7-(2-naphthyl)-3H-imidazo[4,5-b]pyridine
7-(2-naphthyl)-3-(tetrahydro-2H-pyran-4-yl)-3H-imidazo[4,5-b]pyridine
2-(3-bromophenyl)-6-(2-naphthyl)-3-piperidin-4-yl-3H-imidazo[4,5-b]pyridine
6-(2-naphthyl)-3-piperidin-4-yl-2-{3-[3-(trifluoromethyl)phenoxy]phenyl}-3H-imidazo[4,5-b]pyridine
6-(2-naphthyl)-3-piperidin-4-yl-2-(3-thienyl)-3H-imidazo[4,5-b]pyridine
2-(4,5-dimethyl-2-furyl)-6-(2-naphthyl)-3-piperidin-4-yl-3H-imidazo[4,5-b]pyridine
tert-butyl 3-[2-methyl-7-(2-naphthyl)-3H-imidazo[4,5-b]pyridin-3-yl]azetidine-1-carboxylate
tert-butyl (3R)-3-[2-methyl-7-(2-naphthyl)-3H-imidazo[4,5-b]pyridin-3-yl]pyrrolidine-1-carboxylate
1-methyl-7-(2-naphthyl)-3-piperidin-4-yl-1,3-dihydro-2H-imidazo[4,5-b]pyridin-2-one
2-[6-(2-naphthyl)-3-piperidin-4-yl-3H-imidazo[4,5-b]pyridin-2-yl]phenol
6-(2-naphthyl)-3-piperidin-4-yl-2-(3-vinylphenyl)-3H-imidazo[4,5-b]pyridine
2-(2-methoxy-1-naphthyl)-6-(2-naphthyl)-3-piperidin-4-yl-3H-imidazo[4,5-b]pyridine
2-(2,3-difluorophenyl)-6-(2-naphthyl)-3-piperidin-4-yl-3H-imidazo[4,5-b]pyridine
2-(4-chlorophenyl)-6-(2-naphthyl)-3-piperidin-4-yl-3H-imidazo[4,5-b]pyridine
6-(2-naphthyl)-2-(5-nitro-2-thienyl)-3-piperidin-4-yl-3H-imidazo[4,5-b]pyridine
6-(2-naphthyl)-2-(1-oxidopyridin-4-yl)-3-piperidin-4-yl-3H-imidazo[4,5-b]pyridine
7-(2-naphthyl)-3-piperidin-4-yl-1,3-dihydro-2H-imidazo[4,5-b]pyridine-2-thione
tert-butyl (3S)-3-[7-(2-naphthyl)-3H-imidazo[4,5-b]pyridin-3-yl]piperidine-1-carboxylate
tert-butyl 4-{[7-(2-naphthyl)-3H-imidazo[4,5-b]pyridin-3-yl]methyl}piperidine-1-carboxylate
tert-butyl (3S)-3-[2-methyl-7-(2-naphthyl)-3H-imidazo[4,5-b]pyridin-3-yl]pyrrolidine-1-carboxylate
2,2,6,6-tetramethyl-4-[7-(2-naphthyl)-3H-imidazo[4,5-b]pyridin-3-yl]piperidine-1-carbaldehyde
2-methoxy-4-[6-(2-naphthyl)-3-piperidin-4-yl-3H-imidazo[4,5-b]pyridin-2-yl]phenol
2-(2-bromophenyl)-6-(2-naphthyl)-3-piperidin-4-yl-3H-imidazo[4,5-b]pyridine
2-(4-bromophenyl)-6-(2-naphthyl)-3-piperidin-4-yl-3H-imidazo[4,5-b]pyridine
6-(2-naphthyl)-2-(3-phenoxyphenyl)-3-piperidin-4-yl-3H-imidazo[4,5-b]pyridine
2-(3-fluorophenyl)-6-(2-naphthyl)-3-piperidin-4-yl-3H-imidazo[4,5-b]pyridine
2-(1-methyl-1H-imidazol-2-yl)-6-(2-naphthyl)-3-piperidin-4-yl-3H-imidazo[4,5-b]pyridine
2-(1-methyl-1H-pyrrol-2-yl)-6-(2-naphthyl)-3-piperidin-4-yl-3H-imidazo[4,5-b]pyridine
2-(2-chloro-4-methyl-1-phenyl-1H-pyrrol-3-yl)-6-(2-naphthyl)-3-piperidin-4-yl-3H-imidazo[4,5-
b]pyridine
3-[6-(2-naphthyl)-3-piperidin-4-yl-3H-imidazo[4,5-b]pyridin-2-yl]quinoline
2-(3-ethylphenyl)-6-(2-naphthyl)-3-piperidin-4-yl-3H-imidazo[4,5-b]pyridine

In one aspect, the invention provides compounds of the Formula (A₀):

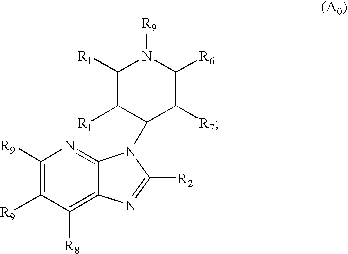

or tautomers thereof or pharmaceutically acceptable salts thereof, wherein R₁, R₂, R₆, R₇, R₈, and R₉ are as defined above for Formula (A₀).

In one embodiment, R₂ is selected from the group consisting of cyclopentyl, cyclohexyl, phenyl, methyl, ethyl, propyl, butyl, iso-butyl, benzodioxolyl, benzodioxinyl, benzothienyl, benzofuranyl, thienyl, pyrimidinyl, imidazolyl, indolyl, furyl, pyrrolyl, pyridinyl, and quinolinyl.

In one embodiment, R₂ is cycloalkyl. In one embodiment, R₂ is C₁-C₆ alkyl. In one embodiment, R₂ is fused aryl-heterocyclyl. In one embodiment, R₂ is benzodioxolyl. In one embodiment, R₂ is benzodioxinyl. In one embodiment, R₂ is heteroaryl. In one embodiment, R₂ is benzothienyl. In one embodiment, R₂ is benzofuranyl. In one embodiment, R₂ is thienyl. In one embodiment, R₂ is furyl. In one embodiment, R₂ is indolyl. In one embodiment, R₂ is pyrrolyl. In one embodiment, R₂ is pyridinyl. In one embodiment, R₂ is quinolinyl. In one embodiment, R₂ is imidazolyl. In one embodiment, R₂ is pyrimidinyl.

Illustrative compounds of Formula A₀ are exemplified by the compounds in the following table:

Compound

7-(2-naphthyl)-3-piperidin-4-yl-3H-imidazo[4,5-b]pyridine
2-methyl-7-(2-naphthyl)-3-piperidin-4-yl-3H-imidazo[4,5-b]pyridine
2-(2-methoxy-1-naphthyl)-7-(2-naphthyl)-3-piperidin-4-yl-3H-imidazo[4,5-b]pyridine
2-(1-ethyl-5-methyl-1H-imidazol-4-yl)-7-(2-naphthyl)-3-piperidin-4-yl-3H-imidazo[4,5-b]pyridine
2-(1-methyl-1H-imidazol-2-yl)-7-(2-naphthyl)-3-piperidin-4-yl-3H-imidazo[4,5-b]pyridine
2-(4-methyl-1H-imidazol-5-yl)-7-(2-naphthyl)-3-piperidin-4-yl-3H-imidazo[4,5-b]pyridine
2-(1H-imidazol-2-yl)-7-(2-naphthyl)-3-piperidin-4-yl-3H-imidazo[4,5-b]pyridine
2-(2-methyl-1H-imidazol-4-yl)-7-(2-naphthyl)-3-piperidin-4-yl-3H-imidazo[4,5-b]pyridine
7-(2-naphthyl)-2-(5-nitro-2-thienyl)-3-piperidin-4-yl-3H-imidazo[4,5-b]pyridine
7-(2-naphthyl)-3-piperidin-4-yl-2-(3-thienyl)-3H-imidazo[4,5-b]pyridine
2-(1-benzothien-3-yl)-7-(2-naphthyl)-3-piperidin-4-yl-3H-imidazo[4,5-b]pyridine
2-(5-methoxy-1H-indol-3-yl)-7-(2-naphthyl)-3-piperidin-4-yl-3H-imidazo[4,5-b]pyridine
2-(5-methyl-1H-indol-3-yl)-7-(2-naphthyl)-3-piperidin-4-yl-3H-imidazo[4,5-b]pyridine
2-(1-acetyl-1H-indol-3-yl)-7-(2-naphthyl)-3-piperidin-4-yl-3H-imidazo[4,5-b]pyridine
2-(4,5-dimethyl-2-furyl)-7-(2-naphthyl)-3-piperidin-4-yl-3H-imidazo[4,5-b]pyridine
7-(2-naphthyl)-2-(1-oxidopyridin-4-yl)-3-piperidin-4-yl-3H-imidazo[4,5-b]pyridine
2-(3,5-dichloropyridin-4-yl)-7-(2-naphthyl)-3-piperidin-4-yl-3H-imidazo[4,5-b]pyridine
2-(1-methyl-1H-pyrrol-2-yl)-7-(2-naphthyl)-3-piperidin-4-yl-3H-imidazo[4,5-b]pyridine
5-[7-(2-naphthyl)-3-piperidin-4-yl-3H-imidazo[4,5-b]pyridine-2-yl]pyrimidine-2,4(1H,3H)-dione
2-(benzo[1,3]dioxol-6-yl)-7-(2-naphthyl)-3-piperidin-4-yl-3H-imidazo[4,5-b]pyridine
2-(2,3-dihydro-1,4-benzodioxin-6-yl)-7-(2-naphthyl)-3-piperidin-4-yl-3H-imidazo[4,5-b]pyridine
2-(2-chloro-4-methyl-1-phenyl-1H-pyrrol-3-yl)-7-(2-naphthyl)-3-piperidin-4-yl-3H-imidazo[4,5-
b]pyridine
2-chloro-3-[7-(2-naphthyl)-3-piperidin-4-yl-3H-imidazo[4,5-b]pyridine-2-yl]quinoline
3-[7-(2-naphthyl)-3-piperidin-4-yl-3H-imidazo[4,5-b]pyridine-2-yl]quinoline
2-[7-(2-naphthyl)-3-piperidin-4-yl-3H-imidazo[4,5-b]pyridine-2-yl]quinoline
2-cyclopentyl-7-(2-naphthyl)-3-piperidin-4-yl-3H-imidazo[4,5-b]pyridine
7-(2-naphthyl)-3-piperidin-4-yl-2-pyridin-2-yl-3H-imidazo[4,5-b]pyridine
7-(2-naphthyl)-3-piperidin-4-yl-2-pyridin-3-yl-3H-imidazo[4,5-b]pyridine
7-(2-naphthyl)-3-piperidin-4-yl-2-pyridin-4-yl-3H-imidazo[4,5-b]pyridine
7-(2-naphthyl)-3-piperidin-4-yl-2-(1H-pyrrol-2-yl)-3H-imidazo[4,5-b]pyridine
2-(1-methyl-1H-indol-2-yl)-7-(2-naphthyl)-3-piperidin-4-yl-3H-imidazo[4,5-b]pyridine
7-(2-naphthyl)-3-piperidin-4-yl-2-(2-thienyl)-3H-imidazo[4,5-b]pyridine
9-[7-(2-naphthyl)-3-piperidin-4-yl-3H-imidazo[4,5-b]pyridine-2-yl]quinoline
6-[7-(2-naphthyl)-3-piperidin-4-yl-3H-imidazo[4,5-b]pyridine-2-yl]quinoline
2-(1-benzofuran-5-yl)-7-(2-naphthyl)-3-piperidin-4-yl-3H-imidazo[4,5-b]pyridine
2-(5-methyl-2-furyl)-7-(2-naphthyl)-3-piperidin-4-yl-3H-imidazo[4,5-b]pyridine
2-(1-methyl-1H-indol-3-yl)-7-(2-naphthyl)-3-piperidin-4-yl-3H-imidazo[4,5-b]pyridine
2-(2-methoxypyridin-3-yl)-7-(2-naphthyl)-3-piperidin-4-yl-3H-imidazo[4,5-b]pyridine
4-[7-(2-naphthyl)-3-piperidin-4-yl-3H-imidazo[4,5-b]pyridine-2-yl]quinoline
2-(2-furyl)-7-(2-naphthyl)-3-piperidin-4-yl-3H-imidazo[4,5-b]pyridine
2-(1-benzofuran-2-yl)-7-(2-naphthyl)-3-piperidin-4-yl-3H-imidazo[4,5-b]pyridine
2-(1-benzothien-2-yl)-7-(2-naphthyl)-3-piperidin-4-yl-3H-imidazo[4,5-b]pyridine
2-isobutyl-7-(2-naphthyl)-3-piperidin-4-yl-3H-imidazo[4,5-b]pyridine
2-cyclohexyl-7-(2-naphthyl)-3-piperidin-4-yl-3H-imidazo[4,5-b]pyridine
7-(2-naphthyl)-3-piperidin-4-yl-2-propyl-3H-imidazo[4,5-b]pyridine
2-butyl-7-(2-naphthyl)-3-piperidin-4-yl-3H-imidazo[4,5-b]pyridine
2-ethyl-7-(2-naphthyl)-3-piperidin-4-yl-3H-imidazo[4,5-b]pyridine
2-(1-naphthyl)-7-(2-naphthyl)-3-piperidin-4-yl-3H-imidazo[4,5-b]pyridine
2,7-di-2-naphthyl-3-piperidin-4-yl-3H-imidazo[4,5-b]pyridine

In one embodiment, the compound of Formula A₀ is 7-(naphthalen-2-yl)-3-(piperidin-4-yl)-3H-imidazo[4,5-b]pyridine.

In another aspect the invention provides compounds of the formula (A₁):

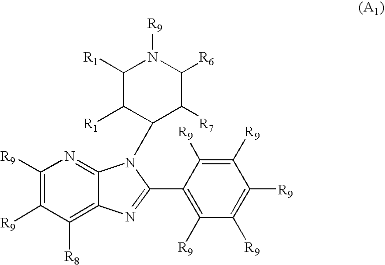

and tautomers thereof or pharmaceutically acceptable salts thereof, wherein R₁, R₆, R₇, R₈, and R₉ are as defined above as that for Formula (A₀).
Illustrative compounds of Formula A₁ are exemplified by the compounds in the following table:

Compound

4-[7-(2-naphthyl)-3-piperidin-4-yl-3H-imidazo[4,5-b]pyridin-2-yl]phenol
3-[7-(2-naphthyl)-3-piperidin-4-yl-3H-imidazo[4,5-b]pyridin-2-yl]phenol
2-[7-(2-naphthyl)-3-piperidin-4-yl-3H-imidazo[4,5-b]pyridin-2-yl]phenol
3-[7-(2-naphthyl)-3-piperidin-4-yl-3H-imidazo[4,5-b]pyridin-2-yl]benzene-1,2-diol
2-methoxy-4-[7-(2-naphthyl)-3-piperidin-4-yl-3H-imidazo[4,5-b]pyridin-2-yl]phenol
3-[7-(2-naphthyl)-3-piperidin-4-yl-3H-imidazo[4,5-b]pyridin-2-yl]-4-nitrophenol
2-(4-methoxyphenyl)-7-(2-naphthyl)-3-piperidin-4-yl-3H-imidazo[4,5-b]pyridine
2-(2-bromophenyl)-7-(2-naphthyl)-3-piperidin-4-yl-3H-imidazo[4,5-b]pyridine
2-(3-bromophenyl)-7-(2-naphthyl)-3-piperidin-4-yl-3H-imidazo[4,5-b]pyridine
2-(4-bromophenyl)-7-(2-naphthyl)-3-piperidin-4-yl-3H-imidazo[4,5-b]pyridine
7-(2-naphthyl)-3-piperidin-4-yl-2-(3-vinylphenyl)-3H-imidazo[4,5-b]pyridine
4-[7-(2-naphthyl)-3-piperidin-4-yl-3H-imidazo[4,5-b]pyridin-2-yl]-N,N-diphenylaniline
7-(2-naphthyl)-2-(3-phenoxyphenyl)-3-piperidin-4-yl-3H-imidazo[4,5-b]pyridine
7-(2-naphthyl)-3-piperidin-4-yl-2-{3-[3-(trifluoromethyl)phenoxy]phenyl}-3H-imidazo[4,5-b]pyridine
7-(2-naphthyl)-3-piperidin-4-yl-2-[4-(trifluoromethyl)phenyl]-3H-imidazo[4,5-b]pyridine
7-(2-naphthyl)-3-piperidin-4-yl-2-[3-(trifluoromethyl)phenyl]-3H-imidazo[4,5-b]pyridine
7-(2-naphthyl)-3-piperidin-4-yl-2-[2-(trifluoromethyl)phenyl]-3H-imidazo[4,5-b]pyridine
2-(2,6-difluorophenyl)-7-(2-naphthyl)-3-piperidin-4-yl-3H-imidazo[4,5-b]pyridine
2-(2,3-difluorophenyl)-7-(2-naphthyl)-3-piperidin-4-yl-3H-imidazo[4,5-b]pyridine
7-(2-naphthyl)-3-piperidin-4-yl-2-[4-(trifluoromethoxy)phenyl]-3H-imidazo[4,5-b]pyridine
7-(2-naphthyl)-3-piperidin-4-yl-2-[2-(trifluoromethoxy)phenyl]-3H-imidazo[4,5-b]pyridine
2-[2,5-bis(trifluoromethyl)phenyl]-7-(2-naphthyl)-3-piperidin-4-yl-3H-imidazo[4,5-b]pyridine
2-[4-fluoro-2-(trifluoromethyl)phenyl]-7-(2-naphthyl)-3-piperidin-4-yl-3H-imidazo[4,5-b]pyridine
2-[2-chloro-3-(trifluoromethyl)phenyl]-7-(2-naphthyl)-3-piperidin-4-yl-3H-imidazo[4,5-b]pyridine
2-(3-fluorophenyl)-7-(2-naphthyl)-3-piperidin-4-yl-3H-imidazo[4,5-b]pyridine
2-(2-chlorophenyl)-7-(2-naphthyl)-3-piperidin-4-yl-3H-imidazo[4,5-b]pyridine
2-(4-fluorophenyl)-7-(2-naphthyl)-3-piperidin-4-yl-3H-imidazo[4,5-b]pyridine
4-[7-(2-naphthyl)-3-piperidin-4-yl-3H-imidazo[4,5-b]pyridine-2-yl]benzonitrile
N-{4-[7-(2-naphthyl)-3-piperidin-4-yl-3H-imidazo[4,5-b]pyridine-2-yl]phenyl}acetamide
7-(2-naphthyl)-2-(4-nitrophenyl)-3-piperidin-4-yl-3H-imidazo[4,5-b]pyridine
2-(4-chlorophenyl)-7-(2-naphthyl)-3-piperidin-4-yl-3H-imidazo[4,5-b]pyridine
7-(2-naphthyl)-3-piperidin-4-yl-2-[3-(trifluoromethoxy)phenyl]-3H-imidazo[4,5-b]pyridine
2-(4-morpholin-4-yl-3-nitrophenyl)-7-(2-naphthyl)-3-piperidin-4-yl-3H-imidazo[4,5-b]pyridine
2-(4-isopropylphenyl)-7-(2-naphthyl)-3-piperidin-4-yl-3H-imidazo[4,5-b]pyridine
2-(4-tert-butylphenyl)-7-(2-naphthyl)-3-piperidin-4-yl-3H-imidazo[4,5-b]pyridine
2-(3-methoxyphenyl)-7-(2-naphthyl)-3-piperidin-4-yl-3H-imidazo[4,5-b]pyridine
7-(2-naphthyl)-2-(2-nitrophenyl)-3-piperidin-4-yl-3H-imidazo[4,5-b]pyridine
2-(4-methylphenyl)-7-(2-naphthyl)-3-piperidin-4-yl-3H-imidazo[4,5-b]pyridine
7-(2-naphthyl)-2-(3-nitrophenyl)-3-piperidin-4-yl-3H-imidazo[4,5-b]pyridine
7-(2-naphthyl)-2-phenyl-3-piperidin-4-yl-3H-imidazo[4,5-b]pyridine
2-(2-methoxyphenyl)-7-(2-naphthyl)-3-piperidin-4-yl-3H-imidazo[4,5-b]pyridine
2-(3-methylphenyl)-7-(2-naphthyl)-3-piperidin-4-yl-3H-imidazo[4,5-b]pyridine
2-(2,4-dimethylphenyl)-7-(2-naphthyl)-3-piperidin-4-yl-3H-imidazo[4,5-b]pyridine
2-(2,4-dichlorophenyl)-7-(2-naphthyl)-3-piperidin-4-yl-3H-imidazo[4,5-b]pyridine
2-(2,4-difluorophenyl)-7-(2-naphthyl)-3-piperidin-4-yl-3H-imidazo[4,5-b]pyridine
2-(3-chlorophenyl)-7-(2-naphthyl)-3-piperidin-4-yl-3H-imidazo[4,5-b]pyridine
2-(2-fluorophenyl)-7-(2-naphthyl)-3-piperidin-4-yl-3H-imidazo[4,5-b]pyridine

### Methods for Using Imidazopyridine Analogs

The imidazopyridine analogs of the present invention exhibit agonism of the canonical Wnt-β-catenin cellular messaging system and, therefore, can be utilized in order to inhibit abnormal cell growth and/or encourage healthy cell regeneration or healthy cell growth. Thus, the imidazopyridine analogs are effective in the treatment of disorders of the canonical Wnt-β-catenin cellular messaging system, including bone disorders. The imidazopyridine analogs may also be effective to treat other disorders of the canonical Wnt-β-catenin cellular messaging system including, cancer and neurological conditions. In particular, the imidazopyridine analogs of the present invention possess bone anabolic growth properties and have cancer cell growth inhibiting effects and are effective in treating cancers. Types of cancers that may be treated include, but are not limited to, solid cancers and malignant lymphomas, and also, leukemia, skin cancer, bladder cancer, breast cancer, uterus cancer, ovary cancer, prostate cancer, lung cancer, colon cancer, pancreas cancer, renal cancer, gastric cancer, brain tumor. Types of neurological conditions that may be treated include, but are not limited too, peripheral neuropathy, spinal cord injury, Parkinson's disease, memory loss, and Alzheimer's disease.

### Therapeutic Administration

When administered to an animal, the imidazopyridine analogs or tautomers thereof or pharmaceutically acceptable salts of the imidazopyridine analogs can be administered neat or as a component of a composition that comprises a physiologically acceptable carrier or vehicle. A composition of the invention can be prepared using a method comprising admixing the imidazopyridine analogs or a tautomer thereof or pharmaceutically acceptable salt of the imidazopyridine analogs and a physiologically acceptable carrier, excipient, or diluent. Admixing can be accomplished using methods well known for admixing a imidazopyridine analog or a tautomer thereof or pharmaceutically acceptable salt of the imidazopyridine analog and a physiologically acceptable carrier, exipient, or diluent.

The present compositions, comprising imidazopyridine analogs or tautomers thereof or pharmaceutically acceptable salts of the imidazopyridine analogs of the invention can be administered orally. The imidazopyridine analogs or tautomers thereof or pharmaceutically acceptable salts of imidazopyridine analogs of the invention can also be administered by any other convenient route, for example, by infusion or bolus injection, by absorption through epithelial or mucocutaneous linings (e.g., oral, rectal, vaginal, and intestinal mucosa) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravascular (e.g., intra-arterial or intravenous), subcutaneous, intranasal, epidural, oral, sublingual, intracerebral, intravaginal, transdermal, rectal, by inhalation, or topical, particularly to the ears, nose, eyes, or skin. In some instances, administration will result in release of the imidazopyridine analog or a tautomer thereof or pharmaceutically acceptable salt of the imidazopyridine analog into the bloodstream. A suitable mode of administration can be readily determined, and is left to the discretion of the practitioner.

In one embodiment, the imidazopyridine analog or a tautomer thereof or pharmaceutically acceptable salt of the imidazopyridine analog is administered orally.

In another embodiment, the imidazopyridine analog or a tautomer thereof or pharmaceutically acceptable salt of the imidazopyridine analog is administered intravenously.

In another embodiment, the imidazopyridine analog or a tautomer thereof or pharmaceutically acceptable salt of the imidazopyridine analog can be administered locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, the imidazopyridine analog or a tautomer thereof or pharmaceutically acceptable salt of the imidazopyridine analog can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the imidazopyridine analog or a tautomer thereof or pharmaceutically acceptable salt of the imidazopyridine analog can be formulated as a suppository, with traditional binders and excipients such as triglycerides.

In another embodiment, the imidazopyridine analog or a tautomer thereof or pharmaceutically acceptable salt of the imidazopyridine analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the imidazopyridine analog or a tautomer thereof or pharmaceutically acceptable salt of the imidazopyridine analog can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the imidazopyridine analog or a tautomer thereof or pharmaceutically acceptable salt of the imidazopyridine analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the imidazopyridine analog or a tautomer thereof or pharmaceutically acceptable salt of the imidazopyridine analogs is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The imidazopyridine analog or tautomer thereof or pharmaceutically acceptable salt of the imidazopyridine analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995, the disclosure of which is herein incorporated by reference).

In one embodiment, the imidazopyridine analog or a tautomer thereof or pharmaceutically acceptable salt of the imidazopyridine analog is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided imidazopyridine analog or tautomer thereof or pharmaceutically acceptable salt of the imidazopyridine analog. In tablets, the imidazopyridine analog or tautomer thereof or pharmaceutically acceptable salt of the imidazopyridine analog is mixed with a carrier having the necessary compression properties in suitable proportions and compacted in the shape and size desired. The powders and tablets can contain about 0.01% to 99% of the imidazopyridine analog or tautomer thereof or pharmaceutically acceptable salt of the imidazopyridine analog.

Capsules may contain mixtures of the imidazopyridine analogs or tautomers thereof or pharmaceutically acceptable salts of the imidazopyridine analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a tautomer thereof or pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the imidazopyridine analog or a tautomer thereof or pharmaceutically acceptable salt of the imidazopyridine analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the imidazopyridine analog or a tautomer thereof or pharmaceutically acceptable salt of the imidazopyridine analog is to be administered by infusion, it can be dispensed, for example, with an infusion bottle containing sterile pharmaceutical grade water or saline. Where the imidazopyridine analog or a tautomer thereof or pharmaceutically acceptable salt of the imidazopyridine analog is administered by injection, an ampule of sterile water for injection or saline can be provided so that the ingredients can be mixed prior to administration.

In another embodiment, the imidazopyridine analog or tautomer thereof or pharmaceutically acceptable salt of the imidazopyridine analog can be administered transdermally through the use of a transdermal patch. Transdermal administrations include administrations across the surface of the body and the inner linings of the bodily passages including epithelial and mucosal tissues. Such administrations can be carried out using the present imidazopyridine analogs or tautomers thereof or pharmaceutically acceptable salts of the imidazopyridine analogs, in lotions, creams, foams, patches, suspensions, solutions, and suppositories (e.g., rectal or vaginal).

Transdermal administration can be accomplished through the use of a transdermal patch containing the imidazopyridine analog or tautomer thereof or pharmaceutically acceptable salt of the imidazopyridine analog and a carrier that is inert to the imidazopyridine analog or tautomer thereof or pharmaceutically acceptable salt of the imidazopyridine analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the imidazopyridine analog or tautomer thereof or pharmaceutically acceptable salt of the imidazopyridine analog into the blood stream, such as a semi-permeable membrane covering a reservoir containing the imidazopyridine analog or tautomer thereof or pharmaceutically acceptable salt of the imidazopyridine analog with or without a carrier, or a matrix containing the active ingredient.

The imidazopyridine analogs or tautomers thereof or pharmaceutically acceptable salts of the imidazopyridine analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The imidazopyridine analog or a tautomer thereof or pharmaceutically acceptable salt of the imidazopyridine analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the imidazopyridine analog or a tautomer thereof or pharmaceutically acceptable salt of the imidazopyridine analog, and can thus reduce the occurrence of adverse side effects.

Controlled- or sustained-release compositions can initially release an amount of the imidazopyridine analog or a tautomer thereof or pharmaceutically acceptable salt of the imidazopyridine analog that promptly produces the desired therapeutic or prophylactic effect, and gradually and continually release other amounts of the imidazopyridine analog or a tautomer thereof or pharmaceutically acceptable salt of the imidazopyridine analog to maintain this level of therapeutic or prophylactic effect over an extended period of time. To maintain a constant level of the imidazopyridine analog or a tautomer thereof or pharmaceutically acceptable salt of the imidazopyridine analog in the body, the imidazopyridine analog or a tautomer thereof or pharmaceutically acceptable salt of the imidazopyridine analog can be released from the dosage form at a rate that will replace the amount of the imidazopyridine analog or a tautomer thereof or pharmaceutically acceptable salt of the imidazopyridine analog being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

In certain embodiments, the present invention is directed to prodrugs of the imidazopyridine analogs or tautomers thereof or pharmaceutically acceptable salts of imidazopyridine analogs of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975). The amount of the imidazopyridine analog or a tautomer thereof or pharmaceutically acceptable salt of the imidazopyridine analog that is effective for treating or preventing a canonical Wnt-β-catenin cellular messaging system-related disorder can be determined using standard clinical techniques. In addition, in vitro or in vivo assays can optionally be employed to help identify suitable dosage ranges. The precise dose to be employed can also depend on the route of administration, the condition, the seriousness of the condition being treated, as well as various physical factors related to the individual being treated, and can be decided according to the judgment of an ordinarily skilled health-care practitioner. The typical dose will range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be readily determined according to the judgment of an ordinarily skilled health-care practitioner. The effective dosage amounts described herein refer to total amounts administered; that is, if more than one imidazopyridine analog or a tautomer thereof or pharmaceutically acceptable salt of the imidazopyridine analog is administered, the effective dosage amounts correspond to the total amount administered.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, pre-filled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The imidazopyridine analog or a tautomer thereof or pharmaceutically acceptable salt of the imidazopyridine analog can be assayed in vitro or in vivo for the desired therapeutic or prophylactic activity prior to use in humans. Animal model systems can be used to demonstrate safety and efficacy.

The present methods for treating or preventing a canonical Wnt-β-catenin cellular messaging system-related disorder, can further comprise administering another therapeutic agent to the animal being administered the imidazopyridine analog or a tautomer thereof or pharmaceutically acceptable salt of the imidazopyridine analog.

Effective amounts of the other therapeutic agents are well known to those skilled in the art. However, it is well within the skilled artisan's purview to determine the other therapeutic agent's optimal effective amount range. The imidazopyridine analog or a tautomer thereof or pharmaceutically acceptable salt of the imidazopyridine analog and the other therapeutic agent can act additively or, in one embodiment, synergistically. In one embodiment, of the invention, where another therapeutic agent is administered to an animal, the effective amount of the imidazopyridine analog or a tautomer thereof or pharmaceutically acceptable salt of the imidazopyridine analog is less than its effective amount would be where the other therapeutic agent is not administered. In this case, without being bound by theory, it is believed that the imidazopyridine analog or a tautomer thereof or pharmaceutically acceptable salt of the imidazopyridine analog and the other therapeutic agent act synergistically.

Suitable other therapeutic agents useful in the methods and compositions of the present invention include, but are not limited to cancer agents, Alzheimer's agents, bone disorder agents, osteoporosis agents, rheumatoid arthritis agents, osteoarthritis agents, and hormone replacement agents.

Suitable cancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

Suitable Alzheimer's agents useful in the methods and compositions of the present invention include, but are not limited to donepezil, galantamine, memantine, niacin, rivastigmine, and tacrine.

Suitable bone disorder and/or osteoporosis agents useful in the methods and compositions of the present invention include, but are not limited to alendronate, bazedoxifene, calcitonin, clomifene, lasofoxifene, ormeloxifene, raloxifene, tamoxifen, and toremifene.

Suitable rheumatoid arthritis agents useful in the methods and compositions of the present invention include, but are not limited to abatacept, acetaminophen adalimumab, aspirin, auranofin, azathioprine, celecoxib, cyclophosphamide, cyclosporine, diclofenac, etanercept, hydroxychloroquine, ibuprofen, indomethacin, infliximab, ketoprofen, leflunomide, methotrexate, minocycline, nabumetone, naproxen, rituximab, and sulfasalazine.

Suitable osteoarthritis agents useful in the methods and compositions of the present invention include, but are not limited to acetaminophen, aspirin, celecoxib, cortisone, hyaluronic acid, ibuprofen, nabumetone, naproxen, rofecoxib, and valdecoxib.

Suitable hormone replacement therapy agents useful in the methods and compositions of the present invention include, but are not limited to estrogen, estradiol, medroxyprogesterone, norethindrone, and progesterone.

In one embodiment, the imidazopyridine analog or a tautomer thereof or pharmaceutically acceptable salt of the imidazopyridine analog is administered concurrently with another therapeutic agent.

In one embodiment, a composition comprising an effective amount of the imidazopyridine analog or a tautomer thereof or pharmaceutically acceptable salt of the imidazopyridine analog and an effective amount of another therapeutic agent within the same composition can be administered.

In another embodiment, a composition comprising an effective amount of the imidazopyridine analog or a tautomer thereof or pharmaceutically acceptable salt of the imidazopyridine analog and a separate composition comprising an effective amount of another therapeutic agent can be concurrently administered.

In another embodiment, an effective amount of the imidazopyridine analog or a tautomer thereof or pharmaceutically acceptable salt of the imidazopyridine analog is administered prior to or subsequent to administration of an effective amount of another therapeutic agent. In this embodiment, the imidazopyridine analog or a tautomer thereof or pharmaceutically acceptable salt of the imidazopyridine analog is administered while the other therapeutic agent exerts its therapeutic effect, or the other therapeutic agent is administered while the imidazopyridine analog or a tautomer thereof or pharmaceutically acceptable salt of the imidazopyridine analog exerts its preventative or therapeutic effect for treating or preventing a canonical Wnt-β-catenin cellular messaging system-related disorder.

In another embodiment, the pharmaceutically acceptable carrier is suitable for oral administration and the composition comprises an oral dosage form.

The imidazopyridine analogs and tautomers thereof or pharmaceutically acceptable salts of imidazopyridine analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

### Methods of Making Imidazopyridine Analogs

Methods useful for making the imidazopyridine analogs are set forth in the Examples below and generalized in Scheme 1.

The reagents used in the preparation of the compounds of this invention can be either commercially obtained or can be prepared by standard procedures described in the literature. In accordance with this invention, compounds of Formula A₀ are produced by the following reaction scheme.

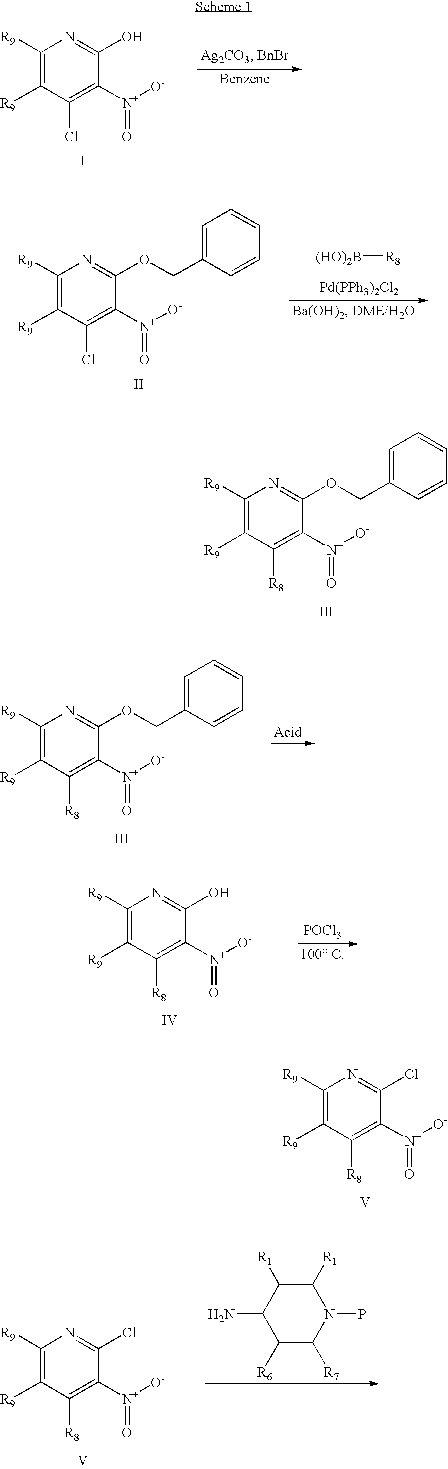

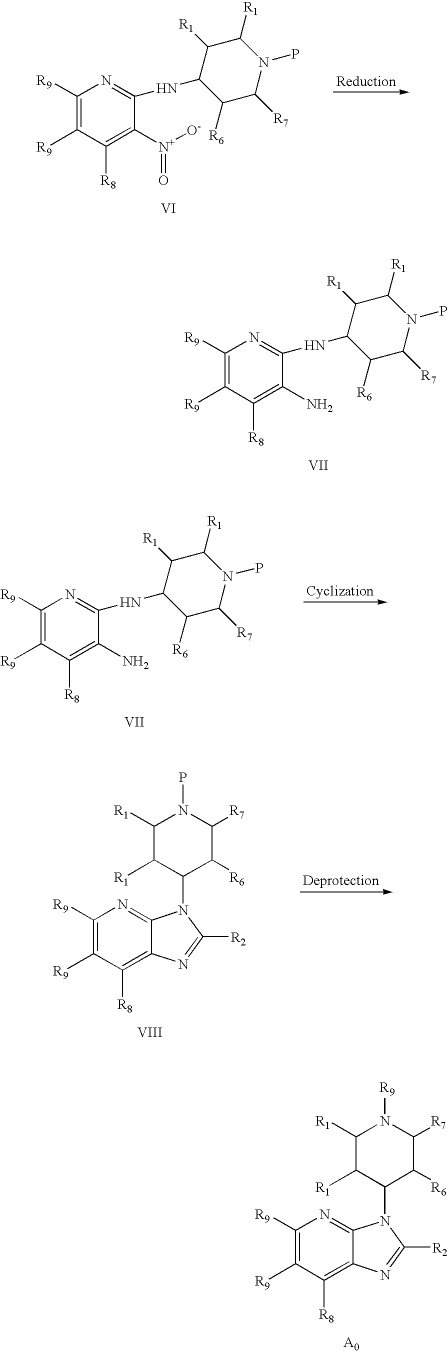

R₁, R₂, R₆, R₇, R₈, and R₉ are as defined above.

Compounds of formula A₀ can be prepared from compounds of formula VIII via any conventional method to effect removal of the t-butyloxycarbonyl amino protecting group (P). In the preferred embodiment of this invention, compounds of formula VIII (where, P=tert-butoxycarbonyl) are treated with trifluoroacetic acid in dichloromethane at room temperature to provide A₀.

Compounds of formula VIII can be prepared from compounds of formula VII via cyclization with any aldehyde, aldehyde equivalent or acid. The reaction is carried out by any conventional method effect cyclization. In the preferred embodiment of this invention, the diamine is treated with triethylorthoformate, triethylorthoacetate, or an aldehyde.

Compounds of formula VII can be prepared from compounds of formula VI via reduction of the nitro functionality to the amine. Any conventional method for reduction of a nitro group may be employed. In accordance with the preferred embodiment of this invention, compounds of formula VI are treated with hydrogen and palladium on carbon in methanol.

Compounds of formula VI can be prepared from compounds of formula V via substitution of the chloro functionality with 4-aminopiperidine suitably protected with the group (P) on the ring nitrogen. This temporary protecting group (P) is employed on the pendant amine to facilitate subsequent successful cyclization to prepare compounds of formula VIII (P=any conventional amine protecting group, for a review of use of suitable amine protecting groups see: Greene, T. W.; Wutts, P. G. M. Protective Groups in Organic Synthesis, 3rd ed.; Wiley and Sons: New York, 1999). In the preferred embodiment of this invention, P=tert-butoxycarbonyl, any conventional method for the deprotection of a carbamate can be utilized at the appropriate step in the synthesis. In the preferred embodiment of this invention the displacement of the chloro functionality with tert-butyl 4-aminopiperidine-1-carboxylate is carried out in acetonitrile or NMP with sodium carbonate and heating to provide compounds of the formula VI.

Compounds of formula V can be prepared from compounds of formula IV by any conventional method for converting an aromatic hydroxyl group to a chloro-functionality. In the preferred embodiment of this invention, this transformation is carried out using phosphorous oxychloride.

Compounds of formula IV can be prepared from compounds of formula III via debenzylation. The conversion of the benzyl ether to the alcohol be accomplished using any conventional method for benzyl group removal. In the preferred embodiment of this invention, compounds of formula III are treated with an aqueous solution of 6N hydrochloric acid and heated at 100° C. to remove the benzyl group.

Compounds of formula III can be prepared from compounds of formula II via a Suzuki reaction. Any conventional method to perform the Suzuki reaction may be employed. In the preferred embodiment of this invention, 2-naphthylboronicacid is used with barium hydroxide in 1,2-dimethoxyethane and water. The catalyst used was dichlorobis(triphenylphosphine)palladium(II) with heating at 100° C.

Compounds of formula II can be prepared as reported previously by Arvanitis et. Al. (Bioorg. Med. Chem. Lett. 2003, 13, 125-128) from compound I.

### EXAMPLES

The following general methods outline the synthesis of the imidazopyridine analogs of the present invention.

### HPLC and LC/MS Methods for the Following Examples and Intermediates

Method A: Column; Xterra MS C18, 5μ, 50×2.1 mm. Mobile phase: 90/10-5/95 water (0.1% formic acid)/acetonitrile (0.1% formic acid), 2 min, hold 1.5 min, 0.8 mL/min., 210-400 nm.
Method B: LC/MS: YMC CombiScreen ProC18 50×4.6 mm I.D. column, S-5 μm, 12 nm. Flow rate 1.0 mL/min. Gradient: 10/90 Acetonitrile/Water (0.1% TFA in both solvents) to 100% acetonitrile over 10 minutes. Hold 100% acetonitrile for 3 mins then back to 10/90 over 2 mins. MS detection using a ThermoFinnigan AQA mass spectrometer in ESI positive mode.
Method C: Column; Xterra RP18, 3.5μ, 150×4.6 mm. Mobile phase: 85/15-5/95 Phosphate buffer (pH=2.1)/ACN+MeOH (1:1) for 10 min, hold 4 mins, 1.2 mL/min., 210-370 nm.
Method D: YMC CombiPrep ProC18 50×20 mm I.D. column, S-5 □m, 12 nm. Flow rate 20 mL/min. Gradient: 10/90 Acetonitrile/Water (0.1% TFA in both solvents) to 100% acetonitrile over 10 minutes then hold for three minutes at 100% acetonitrile and ramp back to 10/90 acetonitrile/water over two minutes.
Method E: Column: Waters Atlantis C18, 5, 2×50 mm. Mobile phase: 95/5-5/95 water (10 mM ammonium acetate)/acetonitrile (10 mM ammonium acetate), 2.5 min., hold 1.5 min., 0.8 mL/min., 210-400 nm.
Method F: Column; Xterra RP18, 3.5μ, 150×4.6 mm. Mobile phase: 85/15-5/95 Ammonium formate buffer (pH=3.5)/ACN+MeOH (1:1) for 10 min, hold 4 mins, 1.2 mL/min., 210-370 nm.
Method G: Column; Xterra RP18, 3.5μ, 150×4.6 mm. Mobile phase: 85/15-5/95 Ammonium bicarbonate buffer (pH=9.5)/ACN+MeOH (1:1) for 10 min, hold 4 mins, 1.2 mL/min., 210-370 nm.
Method H: Column: Waters Atlantis C18, 5μ, 4.6×150 mm. Mobile phase: 95/5-5/95 water (0.1% formic acid)/acetonitrile (0.1% formic acid), 6 min., hold 1.2 min., 1.5 mL/min., 210-400 nm.
Method I: Column: Sunfire prep C18, 5μ, 19×50 mm. Flow rate 20 mL/min. Gradient: 10/90 Acetonitrile/Water to 100% acetonitrile over 10 minutes then hold for three minutes at 100% acetonitrile and ramp back to 10/90 acetonitrile/water over two minutes.
Method J: Waters Gemini C18 50×20 mm I.D. column, S-5 μm, 12 nm. Flow rate 20 mL/min. Gradient: 10/90 Acetonitrile/Water (0.05% ammonia in water) to 100% acetonitrile over 10 minutes then hold for three minutes at 100% acetonitrile and ramp back to 10/90 acetonitrile/water over two minutes.

### Example 1

### 7-(2-naphthyl)-3-piperidin-4-yl-3H-imidazo[4,5-b]pyridine

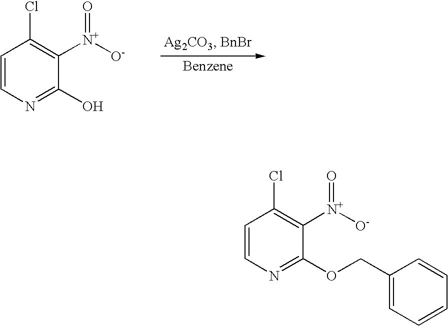

2-(benzyloxy)-4-chloro-3-nitropyridine. A mixture of 4-chloro-3-nitro-2-pyridine (4.0 g, 22.9 mmol), benzylbromide (3.3 mL, 27.5 mmol), and silver carbonate (7.6 g, 27.5 mmol) in benzene (80 mL) was added to a round bottom flask. The flask was wrapped in aluminum foil to keep the reaction in the dark and stirred at room temperature. After 3 days, the mixture was filtered through celite; the celite was washed with benzene and methanol. The filtrate and washings were combined, concentrated, and chromatographed on silica gel (0%-30% ethyl acetate:hexane gradient as the eluent) to yield the benzylated product. 5.1 g (84%). HPLC (method A): Rt=10.3 min. MS: [M+H]⁺=265.

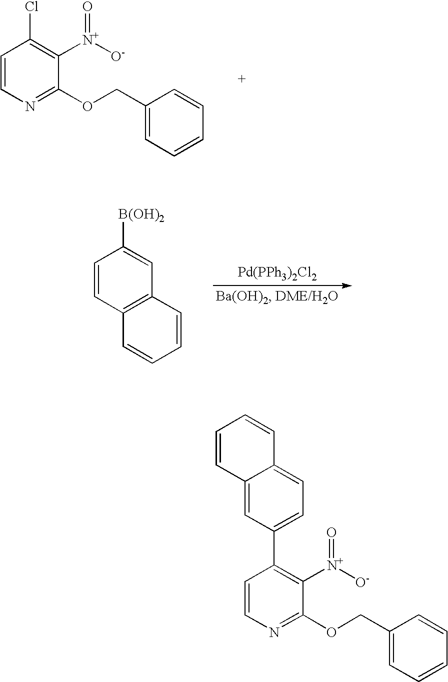

2-(benzyloxy)-4-(2-naphthyl)-3-nitropyridine. To a round bottom flask containing the aryl chloride (8.6 g, 32.6 mmol), 1,2-dimethoxyethane (200 mL), and water (80 mL) was added 2-naphthylboronic acid (5.6 g, 32.6 mmol) and barium hydroxide (10.6 g, 65.2 mmol). Nitrogen was bubbled through the reaction mixture for 10 min. The palladium catalyst was added, and the reaction was heated at 100° C. for 18 h. The reaction was partitioned between ethyl acetate and water and extracted with ethyl acetate (2×). The combined organic layers were washed with water (1×) and brine (1×), dried with MgSO₄, concentrated, and chromatographed on silica gel (10% ethyl acetate:hexane as eluent) to yield the naphylpyridine. 7.5 g (65%). HPLC (method A): Rt=11.5 min. MS: [M+H]⁺=357.

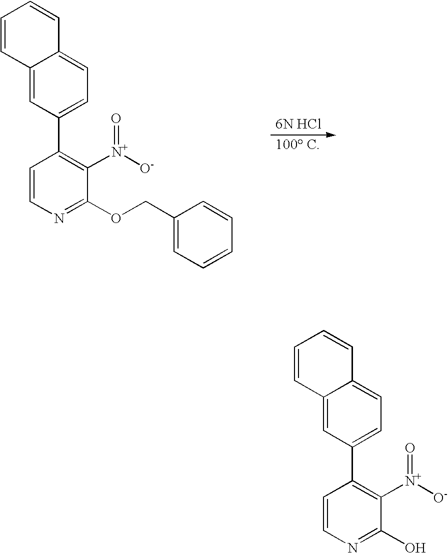

4-(2-naphthyl)-3-nitropyridin-2-ol. A solution of 2-(benzyloxy)-4-(2-naphthyl)-3-nitropyridine (100 mg, 0.28 mmol) in 6 N HCl (5 mL) was heated at 100° C. for 18 h. The reaction was poured into a large Erlenmeyer flask, and ice was added. The solution was neutralized to pH 7 using 6 N KOH and extracted with ethyl acetate (4×). The combined organic extracts were dried with MgSO₄, concentrated, and chromatographed on silica gel (20%-100% ethyl acetate:hexane gradient as the eluent) to yield the de-benzylated material. 43 mg (58%). HPLC (method A): Rt=8.3 min. MS: [M+H]⁺=267.

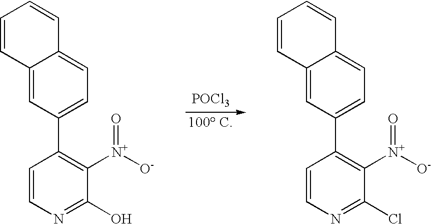

2-chloro-4-(2-naphthyl)-3-nitropyridine. A solution of 4-(2-naphthyl)-3-nitropyridin-2-ol (1.3 g, 4.9 mmol) in phosphorus oxychloride (50 mL) was heated at 100° C. for 3 days. The reaction was cooled to 0° C. The cooled solution was poured slowly onto ice in an oversized Erlenmeyer flask. CAUTION: The phosphorus oxylchloride reacts violently with water/ice. Use extreme care. The aqueous solution was extracted with ethyl acetate (2×), dried with MgSO₄, concentrated, and chromatographed on silica gel (20% ethyl acetate:hexane as eluent) to yield the chloride. 905 mg (65%). HPLC (method A): Rt=10.6 min. MS: [M+H]⁺=285.

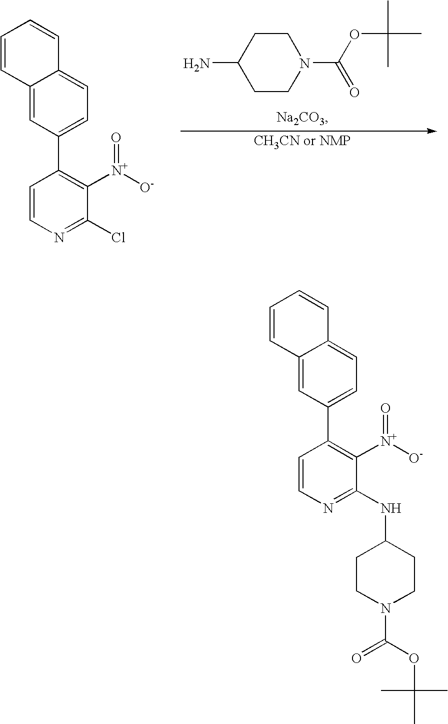

tert-butyl 4-{[4-(2-naphthyl)-3-nitropyridin-2-yl]amino}piperidine-1-carboxylate. To a solution of 2-chloro-4-(2-naphthyl)-3-nitropyridine (3.5 g, 12.3 mmol) and 4-amino-1-boc-piperidine (3.2 g, 16.0 mmol) in acetonitrile (80 mL) was added sodium carbonate (3.3 g, 30.8 mmol). The reaction was heated at 70° C. After 3 weeks, the reaction was 80% complete. Additional 4-amino-1-boc-piperidine (1.7 g, 8.5 mmol) and sodium carbonate (1.7 g, 16.0 mmol) were added, and the reaction was heated at 70° C. for 1 week. (NOTE: Subsequent displacement reactions were performed using NMP as the solvent which lowered the reaction time to 18 h and eliminated the need for additional aliquots of sodium carbonate and boc-piperidine) The reaction was partitioned between ethyl acetate and water and extracted with ethyl acetate (4×). The combined organic extracts were washed with water (1×) and brine (1×), dried with MgSO₄, concentrated, and chromatographed on silica gel (25%-100% ethyl acetate:hexane gradient as the eluent) to yield the piperidinyl pyridine. 5.2 g (94%). HPLC (method A): Rt=11.5 min. MS: [M+H]⁺=449.

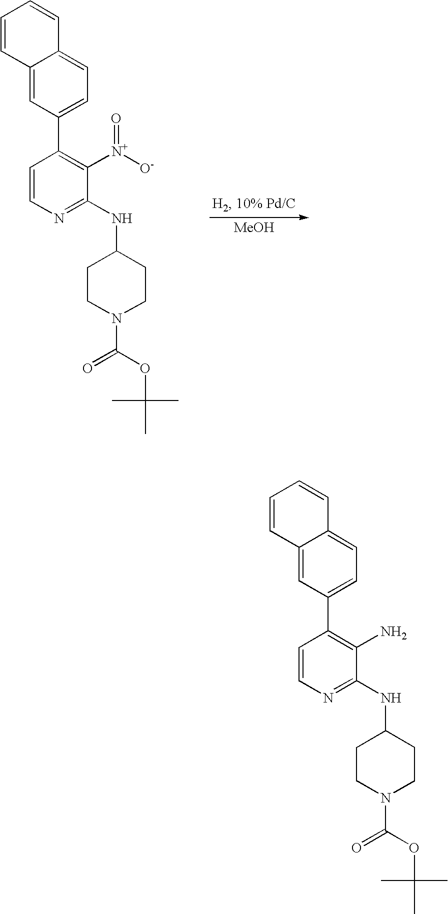

tert-butyl 4-{[3-amino-4-(2-naphthyl)pyridin-2-yl]amino}piperidine-1-carboxylate. A solution of tert-butyl 4-{[4-(2-naphthyl)-3-nitropyridin-2-yl]amino}piperidine-1-carboxylate (600 mg, 1.34 mmol) in methanol (10 mL) was flushed with nitrogen (3×). The palladium catalyst was added, and the reaction was flushed with nitrogen (3×). A hydrogen balloon was affixed to the reaction flask, and the reaction was flushed with hydrogen (3×). After 2 h of stirring at room temperature under a hydrogen atmosphere, the reaction was filtered through celite (2×) and concentrated to yield the pure amine. 533 mg (95%). HPLC (method A): Rt=9.2 min. MS: [M+H]⁺=419.

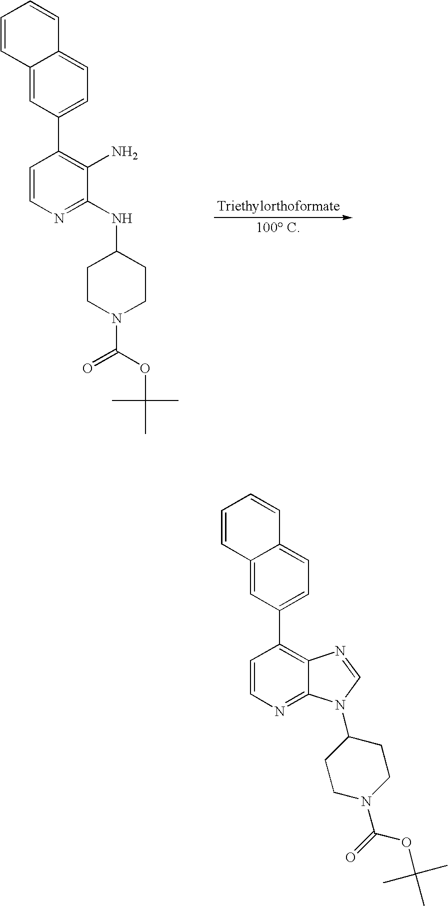

tert-Butyl 4-[7-(2-naphthyl)-3H-imidazo[4,5-b]pyridin-3-yl]piperidine-1-carboxylate. A solution of tert-butyl 4-{[3-amino-4-(2-naphthyl)pyridin-2-yl]amino}piperidine-1-carboxylate (235 mg, 0.562 mmol) in triethylorthoformate (5 mL) was heated at 100° C. for 2 h. The reaction was concentrated to dryness and chromatographed on silica gel (20%-80% ethyl acetate:hexane gradient as the eluent) to yield the cyclized product. 220 mg (94%). HPLC (method A): Rt=10.9 min. MS: [M+H]⁺=429.

tert-butyl 4-(2-methyl-7-(naphthalen-2-yl)-3H-imidazo[4,5-b]pyridin-3-yl)piperidine-1-carboxylate was prepared using triethylorthoacetate in place of triethylorthoformate. HPLC (method A): Rt=11.4 min. MS: [M+H]⁺=443.

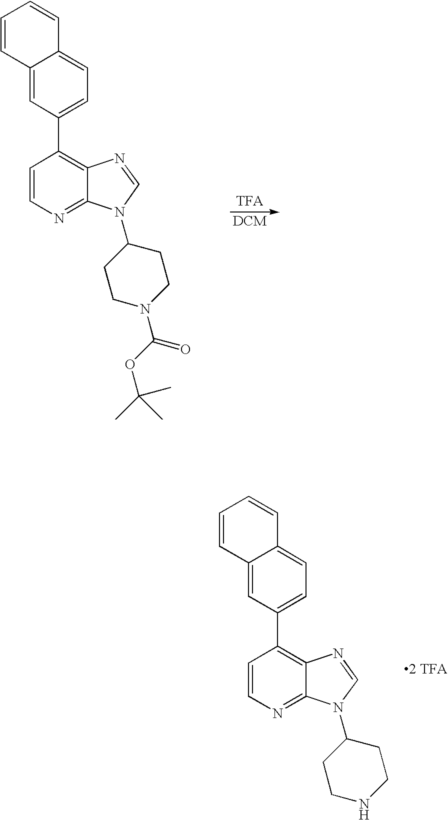

7-(2-naphthyl)-3-piperidin-4-yl-3H-imidazo[4,5-b]pyridine. To a solution of tert-butyl 4-[7-(2-naphthyl)-3H-imidazo[4,5-b]pyridin-3-yl]piperidine-1-carboxylate (3.17 g, 7.40 mmol) in dichloromethane (80 mL) was added trifluoroacetic acid (80 mL). After 2 h of stirring at room temperature, the reaction was concentrated. Toluene was added and the mixture was concentrated again to remove residual TFA. The toluene wash was repeated 4 additional times, and the resulting solid was lyophilized to yield pure piperidine as the di-TFA salt. 4.1 g (99%). HPLC (method A): Rt=7.4 min. MS: [M+H]⁺=329. Compounds in the following table were prepared in the similar way as in Example 1.

| Example | | | R.t | HPLC |
|---|---|---|---|---|
| no. | Compound | MS | (mins.) | Method |
| 1 | 7-(2-naphthyl)-3-piperidin-4-yl-3H-imidazo[4,5-b]pyridine | 329 | 7.4 | A |
| 2 | 2-methyl-7-(2-naphthyl)-3-piperidin-4-yl-3H-imidazo[4,5- | 343 | 9.4 | A |
| | b]pyridine | | | |
| 3 | 2,2,6,6-tetramethyl-4-[7-(2-naphthyl)-3H-imidazo[4,5- | 413 | 10.7 | A |
| | b]pyridin-3-yl]piperidine-1-carbaldehyde | | | |
| 4 | 7-(2-naphthyl)-3-(2,2,6,6-tetramethylpiperidin-4-yl)-3H- | 385 | 8.5 | A |
| | imidazo[4,5-b]pyridine | | | |
| 5 | 7-(2-naphthyl)-3-(tetrahydro-2H-pyran-4-yl)-3H- | 330 | 9.9 | A |
| | imidazo[4,5-b]pyridine | | | |
| 6 | 3-cyclohexyl-7-(2-naphthyl)-3H-imidazo[4,5-b]pyridine | 328 | 11.4 | A |

### Example 7

### tert-butyl 4-[7-(2-naphthyl)-3H-imidazo[4,5-b]pyridin-3-yl]piperidine-1-carboxylate

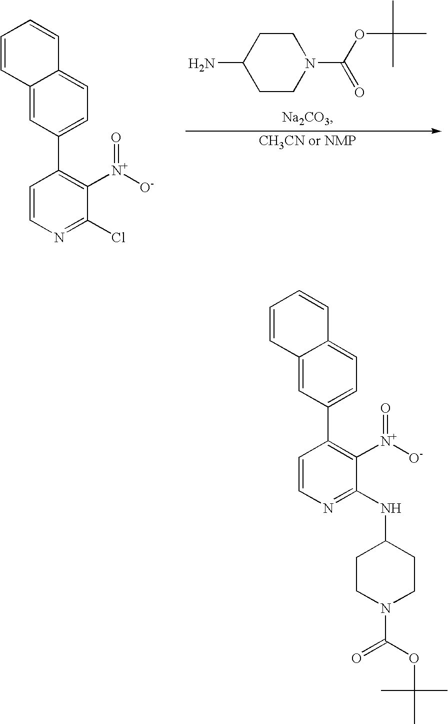

tert-butyl 4-{[4-(2-naphthyl)-3-nitropyridin-2-yl]amino}piperidine-1-carboxylate. To a solution of 2-chloro-4-(2-naphthyl)-3-nitropyridine (3.5 g, 12.3 mmol) and 4-amino-1-boc-piperidine (3.2 g, 16.0 mmol) in acetonitrile (80 mL) was added sodium carbonate (3.3 g, 30.8 mmol). The reaction was heated at 70° C. After 3 weeks, the reaction was 80% complete. Additional 4-amino-1-boc-piperidine (1.7 g, 8.5 mmol) and sodium carbonate (1.7 g, 16.0 mmol) were added, and the reaction was heated at 70° C. for 1 week. (NOTE: Subsequent displacement reactions were performed using NMP as the solvent which lowered the reaction time to 18 h and eliminated the need for additional aliquots of sodium carbonate and boc-piperidine) The reaction was partitioned between ethyl acetate and water and extracted with ethyl acetate (4×). The combined organic extracts were washed with water (1×) and brine (1×), dried with MgSO₄, concentrated, and chromatographed on silica gel (25%-100% ethyl acetate:hexane gradient as the eluent) to yield the piperidinyl pyridine. 5.2 g (94%). HPLC (method A): Rt=11.5 min. MS: [M+H]⁺=449.

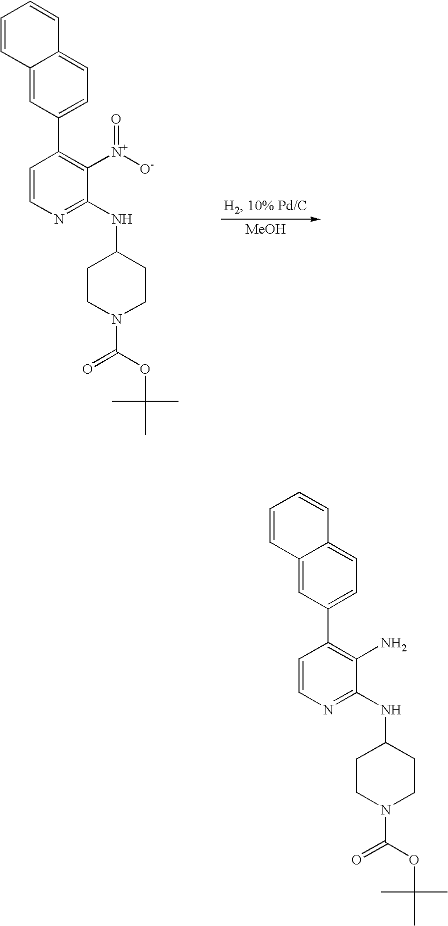

tert-butyl 4-{[3-amino-4-(2-naphthyl)pyridin-2-yl]amino}piperidine-1-carboxylate. A solution of tert-butyl 4-{[4-(2-naphthyl)-3-nitropyridin-2-yl]amino}piperidine-1-carboxylate (600 mg, 1.34 mmol) in methanol (10 mL) was flushed with nitrogen (3×). The palladium catalyst was added, and the reaction was flushed with nitrogen (3×). A hydrogen balloon was affixed to the reaction flask, and the reaction was flushed with hydrogen (3×). After 2 h of stirring at room temperature under a hydrogen atmosphere, the reaction was filtered through celite (2×) and concentrated to yield the pure amine. 533 mg (95%). HPLC (method A): Rt=9.2 min. MS: [M+H]⁺=419.

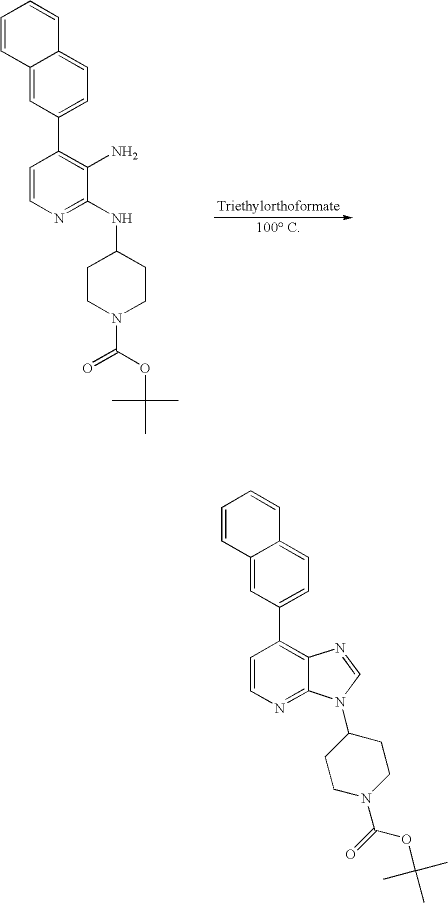

tert-butyl 4-[7-(2-naphthyl)-3H-imidazo[4,5-b]pyridin-3-yl]piperidine-1-carboxylate. A solution of tert-butyl 4-{[3-amino-4-(2-naphthyl)pyridin-2-yl]amino}piperidine-1-carboxylate (235 mg, 0.562 mmol) in triethylorthoformate (5 mL) was heated at 100° C. for 2 h. The reaction was concentrated to dryness and chromatographed on silica gel (20%-80% ethyl acetate:hexane gradient as the eluent) to yield the cyclized product. 220 mg (94%). HPLC (method A): Rt=10.9 min. MS: [M+H]⁺=429. Examples 8-27 in the following table were prepared with the similar procedures as in Example 7. Triethylorthoacetate in place of triethylorthoformate were used in the last step for Examples 17 to 27.

| Example | | | | HPLC |
|---|---|---|---|---|
| no. | Compound | MS | RT (mins.) | Method |
| 8 | tert-butyl 4-[2-methyl-7-(2-naphthyl)-3H-imidazo[4,5- | 443 | 11.4 | A |
| | b]pyridin-3-yl]piperidine-1-carboxylate | | | |
| 11 | tert-butyl 3-[7-(2-naphthyl)-3H-imidazo[4,5-b]pyridin-3- | 401 | 10.9 | A |
| | yl]azetidine-1-carboxylate | | | |
| 12 | tert-butyl (3R)-3-[7-(2-naphthyl)-3H-imidazo[4,5-b]pyridin-3- | 415 | 11.0 | A |
| | yl]pyrrolidine-1-carboxylate | | | |
| 13 | tert-butyl (3S)-3-[7-(2-naphthyl)-3H-imidazo[4,5-b]pyridin-3- | 415 | 11.0 | A |
| | yl]pyrrolidine-1-carboxylate | | | |
| 14 | tert-butyl (3R)-3-[7-(2-naphthyl)-3H-imidazo[4,5-b]pyridin-3- | 429 | 11.3 | A |
| | yl]piperidine-1-carboxylate | | | |
| 15 | tert-butyl (3S)-3-[7-(2-naphthyl)-3H-imidazo[4,5-b]pyridin-3- | 429 | 11.3 | A |
| | yl]piperidine-1-carboxylate | | | |
| 16 | tert-butyl 4-{[7-(2-naphthyl)-3H-imidazo[4,5-b]pyridin-3- | 443 | 11.3 | A |
| | yl]methyl}piperidine-1-carboxylate | | | |
| 19 | tert-butyl 3-[2-methyl-7-(2-naphthyl)-3H-imidazo[4,5-b]pyridin- | 415 | 11.1 | A |
| | 3-yl]azetidine-1-carboxylate | | | |
| 20 | tert-butyl (3R)-3-[2-methyl-7-(2-naphthyl)-3H-imidazo[4,5- | 429 | 11.4 | A |
| | b]pyridin-3-yl]pyrrolidine-1-carboxylate | | | |
| 21 | tert-butyl (3S)-3-[2-methyl-7-(2-naphthyl)-3H-imidazo[4,5- | 429 | 11.4 | A |
| | b]pyridin-3-yl]pyrrolidine-1-carboxylate | | | |
| 22 | tert-butyl (3R)-3-[2-methyl-7-(2-naphthyl)-3H-imidazo[4,5- | 443 | 11.7 | A |
| | b]pyridin-3-yl]piperidine-1-carboxylate | | | |
| 23 | tert-butyl (3S)-3-[2-methyl-7-(2-naphthyl)-3H-imidazo[4,5- | 443 | 11.7 | A |
| | b]pyridin-3-yl]piperidine-1-carboxylate | | | |
| 24 | tert-butyl 4-{[2-methyl-7-(2-naphthyl)-3H-imidazo[4,5-b]pyridin- | 457 | 11.4 | A |
| | 3-yl]methyl}piperidine-1-carboxylate | | | |
| 25 | 3-[3-(1H-imidazol-1-yl)propyl]-7-(2-naphthyl)-3H-imidazo[4,5- | 354 | 1.13 | B |
| | b]pyridine | | | |
| 26 | 3-[2-(4-methylphenyl)ethyl]-7-(2-naphthyl)-3H-imidazo[4,5- | 364 | 2.38 | B |
| | b]pyridine | | | |
| 27 | 2-methyl-3-[2-(4-methylphenyl)ethyl]-7-(2-naphthyl)-3H- | 378 | 2.33 | B |
| | imidazo[4,5-b]pyridine | | | |

### Example 28

### 7-(2-naphthyl)-3-piperidin-4-yl-3H-imidazo[4,5-b]pyridine

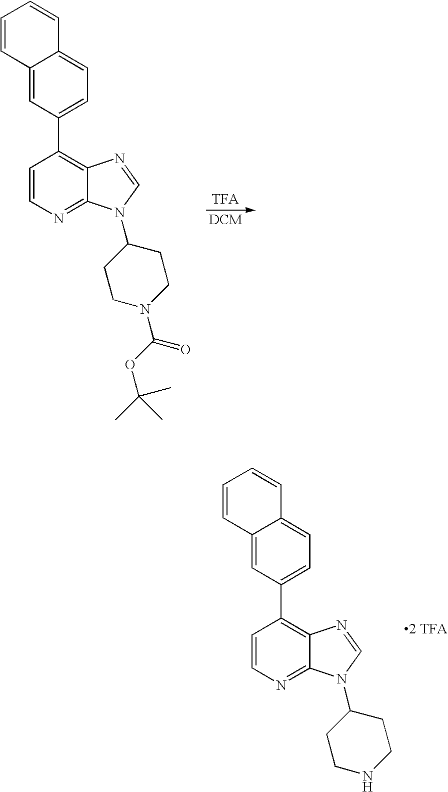

7-(2-naphthyl)-3-piperidin-4-yl-3H-imidazo[4,5-b]pyridine. To a solution of tert-butyl 4-[7-(2-naphthyl)-3H-imidazo[4,5-b]pyridin-3-yl]piperidine-1-carboxylate (3.17 g, 7.40 mmol) in dichloromethane (80 mL) was added trifluoroacetic acid (80 mL). After 2 h of stirring at room temperature, the reaction was concentrated. Toluene was added and the mixture was concentrated again to remove residual TFA. The toluene wash was repeated 4 additional times, and the resulting solid was lyophilized to yield pure piperidine as the di-TFA salt. 4.1 g (99%). HPLC (method A): Rt=7.4 min. MS: [M+H]⁺=329. Examples 29-45 in the following table were prepared in the similar procedures as in Example 28.

| Example | | | RT | HPLC |
|---|---|---|---|---|
| no. | Compound | MS | (mins.) | Method |
| 29 | 2-methyl-7-(2-naphthyl)-3-piperidin-4-yl-3H-imidazo[4,5- | 343 | 9.4 | A |
| | b]pyridine | | | |
| 32 | 3-azetidin-3-yl-7-(2-naphthyl)-3H-imidazo[4,5-b]pyridine | 301 | 1.15 | B |
| 33 | 7-(2-naphthyl)-3-[(3R)-pyrrolidin-3-yl]-3H-imidazo[4,5-b]pyridine | 315 | 1.16 | B |
| 34 | 7-(2-naphthyl)-3-[(3S)-pyrrolidin-3-yl]-3H-imidazo[4,5-b]pyridine | 315 | 1.23 | B |
| 35 | 7-(2-naphthyl)-3-[(3R)-piperidin-3-yl]-3H-imidazo[4,5-b]pyridine | 329 | 1.22 | B |
| 36 | 7-(2-naphthyl)-3-[(3S)-piperidin-3-yl]-3H-imidazo[4,5-b]pyridine | 329 | 1.21 | B |
| 37 | 7-(2-naphthyl)-3-(piperidin-4-ylmethyl)-3H-imidazo[4,5-b]pyridine | 343 | 1.18 | B |
| 40 | 3-azetidin-3-yl-2-methyl-7-(2-naphthyl)-3H-imidazo[4,5-b]pyridine | 315 | 1.19 | B |
| 41 | 2-methyl-7-(2-naphthyl)-3-[(3R)-pyrrolidin-3-yl]-3H-imidazo[4,5- | 329 | 1.21 | B |
| | b]pyridine | | | |
| 42 | 2-methyl-7-(2-naphthyl)-3-[(3S)-pyrrolidin-3-yl]-3H-imidazo[4,5- | 329 | 1.17 | B |
| | b]pyridine | | | |
| 43 | 2-methyl-7-(2-naphthyl)-3-[(3R)-piperidin-3-yl]-3H-imidazo[4,5- | 343 | 1.24 | B |
| | b]pyridine | | | |
| 44 | 2-methyl-7-(2-naphthyl)-3-[(3S)-piperidin-3-yl]-3H-imidazo[4,5- | 343 | 1.24 | B |
| | b]pyridine | | | |
| 45 | 2-methyl-7-(2-naphthyl)-3-(piperidin-4-ylmethyl)-3H-imidazo[4,5- | 357 | 1.18 | B |
| | b]pyridine | | | |

### Example 46

### 3-(1-acetylpiperidin-4-yl)-7-(2-naphthyl)-3H-imidazo[4,5-b]pyridine

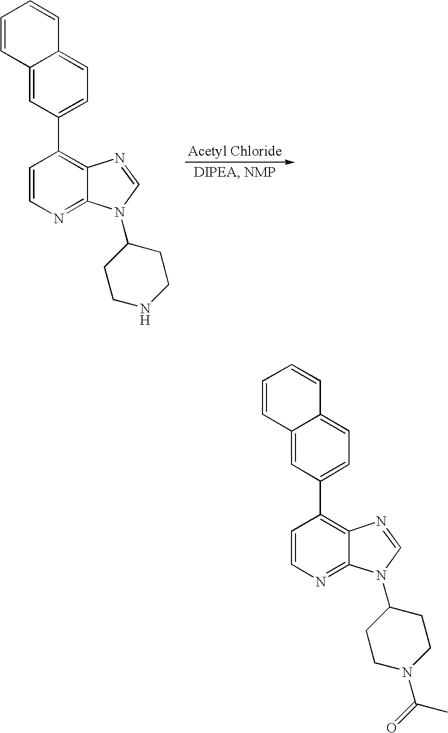

3-(1-acetylpiperidin-4-yl)-7-(2-naphthyl)-3H-imidazo[4,5-b]pyridine. To a solution of 7-(2-naphthyl)-3-piperidin-4-yl-3H-imidazo[4,5-b]pyridine (16 mg, 28.6 μmol) in NMP (1 mL)was added diisopropylethylamine (19.9 μL, 114.4 μmmol) followed by acetyl chloride (8.1 μL, 114 μmmol). The reaction was stirred for 16 hours at room temperature. Water (0.1 mL) was added and the mixture was purified by HPLC (Method D) to yield acylated piperidine. 7.1 mg (66%). HPLC (method B): Rt=1.60 min. MS: [M+H]⁺=371. Examples 47-57 in the following table were prepared in the similar procedures as in Example 46. except that acetyl chloride was substituted by the requisite electrophile.

| Example | | | | HPLC |
|---|---|---|---|---|
| no. | Compound | MS | RT (mins.) | Method |
| 47 | 7-(2-naphthyl)-3-[1-(trifluoroacetyl)piperidin-4-yl]-3H- | 425 | 1.97 | B |
| | imidazo[4,5-b]pyridine | | | |
| 48 | methyl 4-[7-(2-naphthyl)-3H-imidazo[4,5-b]pyridin-3- | 387 | 1.79 | B |
| | yl]piperidine-1-carboxylate | | | |
| 49 | N,N-dimethyl-4-[7-(2-naphthyl)-3H-imidazo[4,5-b]pyridin-3- | 400 | 1.67 | B |
| | yl]piperidine-1-carboxamide | | | |
| 50 | N-ethyl-4-[7-(2-naphthyl)-3H-imidazo[4,5-b]pyridin-3- | 400 | 1.60 | B |
| | yl]piperidine-1-carboxamide | | | |
| 51 | N-isopropyl-4-[7-(2-naphthyl)-3H-imidazo[4,5-b]pyridin-3- | 414 | 1.67 | B |
| | yl]piperidine-1-carboxamide | | | |
| 52 | N-cyclohexyl-4-[7-(2-naphthyl)-3H-imidazo[4,5-b]pyridin-3- | 454 | 1.90 | B |
| | yl]piperidine-1-carboxamide | | | |
| 53 | 3-[1-(morpholin-4-ylcarbonyl)piperidin-4-yl]-7-(2-naphthyl)- | 442 | 1.62 | B |
| | 3H-imidazo[4,5-b]pyridine | | | |
| 54 | N-(3′,6′-dihydroxy-3-oxo-3H-spiro[2-benzofuran-1,9′- | 718 | 1.85 | B |
| | xanthen]-5-yl)-4-[7-(2-naphthyl)-3H-imidazo[4,5-b]pyridin-3- | | | |
| | yl]piperidine-1-carbothioamide | | | |
| 55 | N,N-dimethyl-4-[7-(2-naphthyl)-3H-imidazo[4,5-b]pyridin-3- | 436 | 1.87 | B |
| | yl]piperidine-1-sulfonamide | | | |
| 56 | 3-[1-(methylsulfonyl)piperidin-4-yl]-7-(2-naphthyl)-3H- | 407 | 1.71 | B |
| | imidazo[4,5-b]pyridine | | | |
| 57 | 3-(1-benzylpiperidin-4-yl)-7-(2-naphthyl)-3H-imidazo[4,5- | 419 | 1.27 | B |
| | b]pyridine | | | |

### Example 58

### tert-butyl 4-[5-(2-naphthyl)-3H-imidazo[4,5-b]pyridin-3-yl]piperidine-1-carboxylate

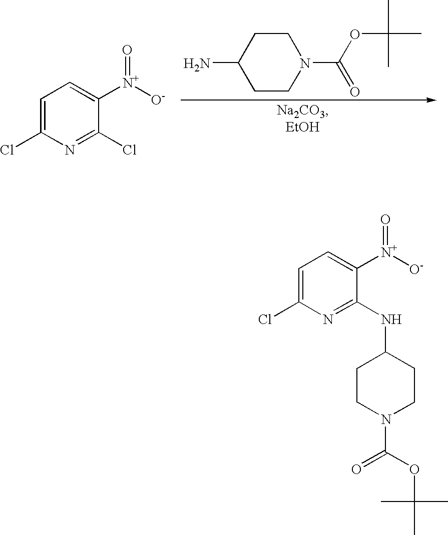

tert-butyl 4-[(6-chloro-3-nitropyridin-2-yl)amino]piperidine-1-carboxylate. To a solution of 2,6-dichloro-3-nitropyridine (600 mg, 3.1 mmol) and sodium carbonate (821 mg, 7.75 mmol) in ethanol (7 mL) at 0° C. was added dropwise 4-amino-1-boc-piperidine (940 mg, 4.65 mmol) in ethanol (0.5 mL). The reaction was warmed to room temperature overnight and concentrated under reduced pressure. The crude was partitioned between ethyl acetate and water and extracted with ethyl acetate. The ethyl acetate layer was washed with brine (1×) and dried with MgSO₄, concentrated, and chromatographed on silica gel (50%-50% ethyl acetate:hexane as the eluent) to yield the titled compound 770 mg (70%) as a yellow solid. HPLC (method A): Rt=10.8 min. MS: [M−H]⁻=355.

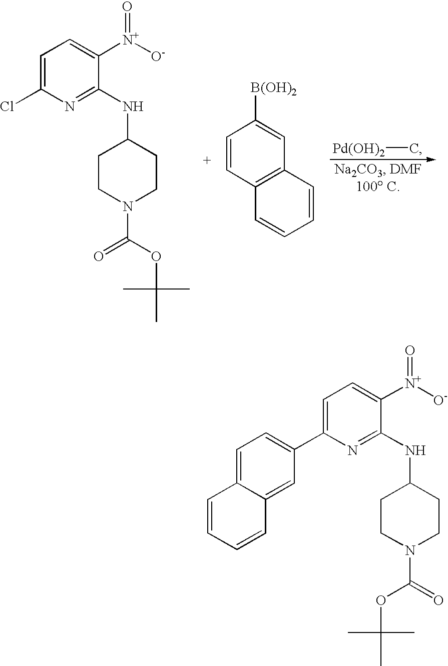

tert-butyl 4-{[6-(2-naphthyl)-3-nitropyridin-2-yl]amino}piperidine-1-carboxylate. To a vial equipped with a teflon-coated cap was added tert-butyl 4-[(6-chloro-3-nitropyridin-2-yl)amino]piperidine-1-carboxylate (320 mg, 0.9 mmol), sodium carbonate (95.4 mg, 0.9 mmol) and 2-naphthylboronic acid (185 mg, 1.08 mmol) in anhydrous DMF (4 mL). Nitrogen was bubbled through the reaction mixture for 10 min. Palladium hydroxide (20% on carbon, 20 mg) was added, and the reaction was sealed and heated at 100° C. for 18 h on a shaker block. The reaction was cooled to room temperature and diluted with ethyl acetate (100 mL) and washed with brine (1×) and water (3×). The ethyl acetate layer was dried over MgSO₄, concentrated, and chromatographed on silica gel (20% ethyl acetate:hexane as eluent) to yield the titled compound 354 mg (88%) as a yellow solid. HPLC (method A): Rt=12.3 min. MS: [M+H-tbutyl]⁺=393.

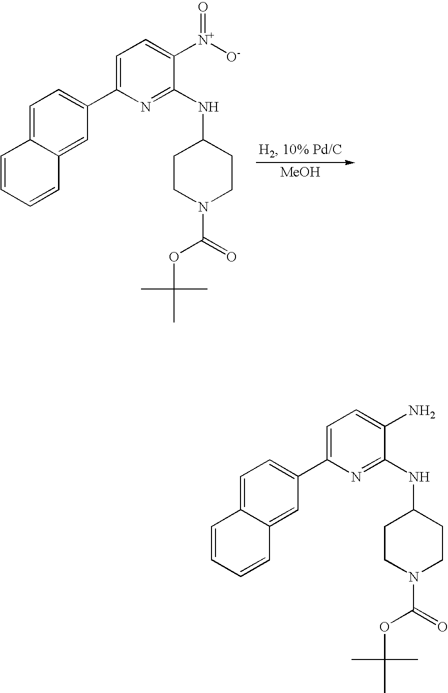

tert-butyl 4-{[3-amino-6-(2-naphthyl)pyridin-2-yl]amino}piperidine-1-carboxylate. A solution of tert-butyl 4-{[6-(2-naphthyl)-3-nitropyridin-2-yl]amino}piperidine-1-carboxylate (354 mg, 789 μmol) in methanol (10 mL) was flushed with nitrogen (3×). The palladium catalyst (10% on carbon, 60 mg) was added, and the reaction was flushed with nitrogen (3×). A hydrogen balloon was affixed to the reaction flask using a 3-way stopcock, and the reaction was flushed with hydrogen (3×) under vacuum. After 1 h of stirring at room temperature under a hydrogen atmosphere, the reaction was filtered through celite with rinsing using methanol (3×10 mL) and ethyl acetate (3×10 mL). The combined filtrate was concentrated to yield the titled amine. The amine was purified by flash chromatography (5% methanol:dichloromethane as eluent) yielding 320 mg (97%) of the desired tan solid. HPLC (method A): Rt=11.3 min. MS: [M+H]⁺=419.

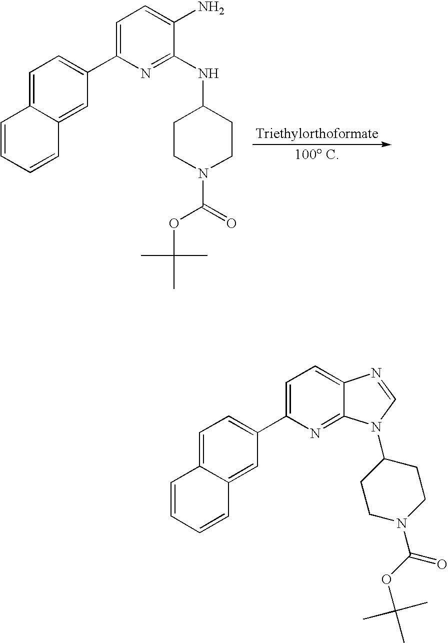

tert-butyl 4-[5-(2-naphthyl)-3H-imidazo[4,5-b]pyridin-3-yl]piperidine-1-carboxylate. A solution of tert-butyl 4-{[3-amino-6-(2-naphthyl)pyridin-2-yl]amino}piperidine-1-carboxylate (150 mg, 0.358 mmol) in triethylorthoformate (5 mL) was heated at 100° C. for 18 h. Analysis of the crude reaction by LC/MS revealed a 1:1 mixture of desired product and diamine starting material. Longer reaction times were unsuccessful at improving this ratio. TLC analysis of the crude using 2.5% MeOH in dichloromethane as the solvent revealed one product (Rf=0.8). Since the LC/MS buffer contained 0.1% formic acid and provided the desired product with starting diamine in a 1:1 ratio, it was envisioned that a pre-cyclized dimer joined by one carbon unit cyclized under the acidic conditions providing the 1:1 ratio. The crude reaction was concentrated to dryness and treated with formic acid: acetonitrile: water (1:1:2, 4 mL) for 30 min. TLC revealed formation of stating material and the new desired product in a 1:1 ratio. The crude reaction was concentrated and purified by RP-HPLC (Method D) to yield the titled product 60 mg (39%) as a white solid. HPLC (method A): Rt=11.2 min. MS: [M+H]⁺=429.

### Example 59

### 5-(2-naphthyl)-3-piperidin-4-yl-3H-imidazo[4,5-b]pyridine

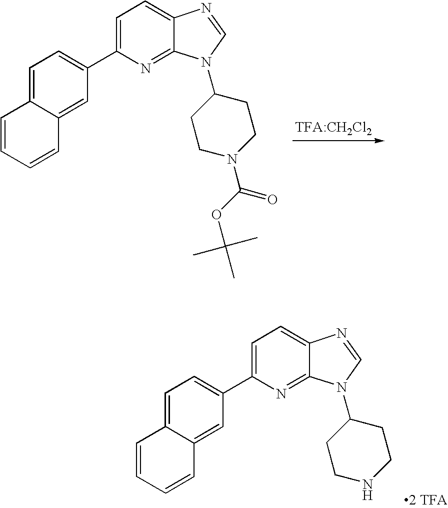

5-(2-naphthyl)-3-piperidin-4-yl-3H-imidazo[4,5-b]pyridine. To a solution of tert-butyl 4-[5-(2-naphthyl)-3H-imidazo[4,5-b]pyridin-3-yl]piperidine-1-carboxylate (14 mg, 32.6 μmol) in dichloromethane (2.5 mL) was added trifluoroacetic acid (2.5 mL). After 1 h of stirring at room temperature, the reaction was concentrated. Toluene was added and the mixture was concentrated again to remove residual TFA. The toluene wash was repeated 4 additional times, and the resulting solid was lyophilized to yield 18 mg of desired tan solid as the di-TFA salt. HPLC (method A): Rt=7.6 min. MS: [M+H]⁺=329.

### Example 60

### tert-butyl 4-[3-methyl-5-(2-naphthyl)-3H-imidazo[4,5-b]pyridin-2-yl]piperidine-1-carboxylate

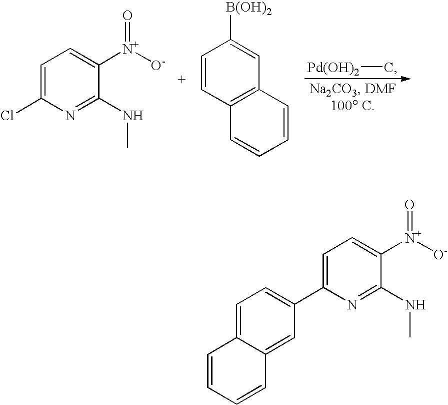

N-methyl-6-(2-naphthyl)-3-nitropyridin-2-amine. To a vial equipped with a teflon-coated cap was added 6-chloro-N-methyl-3-nitropyridin-2-amine (180 mg, 0.96 mmol), sodium carbonate (102 mg, 0.96 mmol) and 2-naphthylboronic acid (198 mg, 1.15 mmol) in anhydrous DMF (5 mL). Nitrogen was bubbled through the reaction mixture for 10 min. Palladium hydroxide (20% on carbon, 13 mg) was added, and the reaction was sealed and heated at 100° C. for 9 h on a shaker block. The reaction was cooled to room temperature and diluted with water (1 mL) and methanol (1 mL). The product precipitated and was collected to yield the titled compound 190 mg (71%) as a gold solid. HPLC (method A): Rt=11.6 min. MS: [M+H-tbutyl]⁺=280.

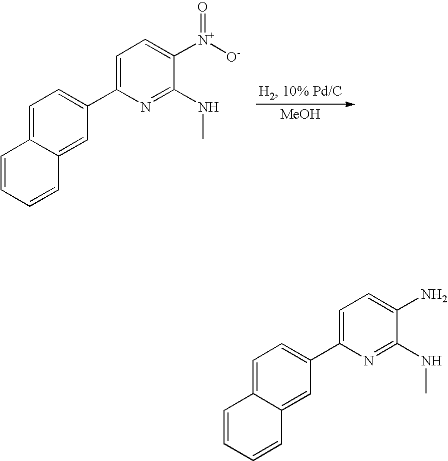

N2-methyl-6-(naphthalen-2-yl)pyridine-2,3-diamine. A solution of N-methyl-6-(2-naphthyl)-3-nitropyridin-2-amine (150 mg, 537 μmol) in methanol (5 mL) was flushed with nitrogen (3×). The palladium catalyst (10% on carbon, 50 mg) was added, and the reaction was flushed with nitrogen (3×). A hydrogen balloon was affixed to the reaction flask using a 3-way stopcock, and the reaction was flushed with hydrogen (3×) under vacuum. After 1 h of stirring at room temperature under a hydrogen atmosphere, the reaction was filtered through celite with rinsing using methanol (3×50 mL). The combined filtrate was concentrated to yield the titled amine 130 mg (93.5%), which was used in the next reaction without further purification. HPLC (method B): Rt=1.17 min. MS: [M+H]⁺=250.

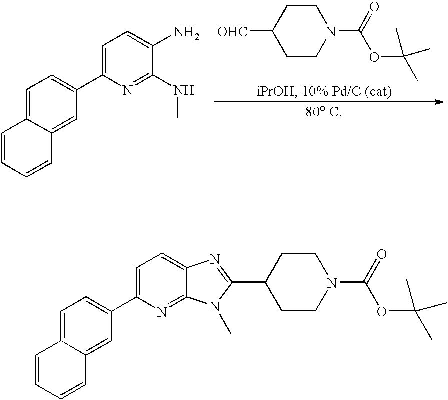

tert-butyl 4-[3-methyl-5-(2-naphthyl)-3H-imidazo[4,5-b]pyridin-2-yl]piperidine-1-carboxylate. A solution of N2-methyl-6-(naphthalen-2-yl)pyridine-2,3-diamine (80 mg, 0.32 mmol) in isopropanol (5 mL) was added to a round bottom flask containing tert-butyl 4-formylpiperidine-1-carboxylate (82 mg, 0.384 mmol) followed by the palladium catalyst (10% on carbon, 5 mg), and the reaction was heated at 80° C. open to ambient atmosphere with stirring over an oil bath for 2 days. Analysis by LC/MS revealed that the reaction was about 20% complete. Additional tert-butyl 4-formylpiperidine-1-carboxylate (164 mg, 0.768 mmol) was added and the reaction was stirred at 80° C. for an additional day. The reaction was then removed from the heat and allowed to cool to room temperature and filtered through Celite. The Celite was rinsed with methanol (3×5 mL) and the combined filtrate was concentrated under reduced pressure. The crude residue was purified by semi-prep HPLC (Method D) to obtain the titled compound (21.8 mg, 15.2%) as a white solid. HPLC (method A): Rt=11.3 min. MS: [M+H]⁺=443.
Alternative procedure for preparation of tert-butyl 4-[3-methyl-5-(2-naphthyl)-3H-imidazo[4,5-b]pyridin-2-yl]piperidine-1-carboxylate.

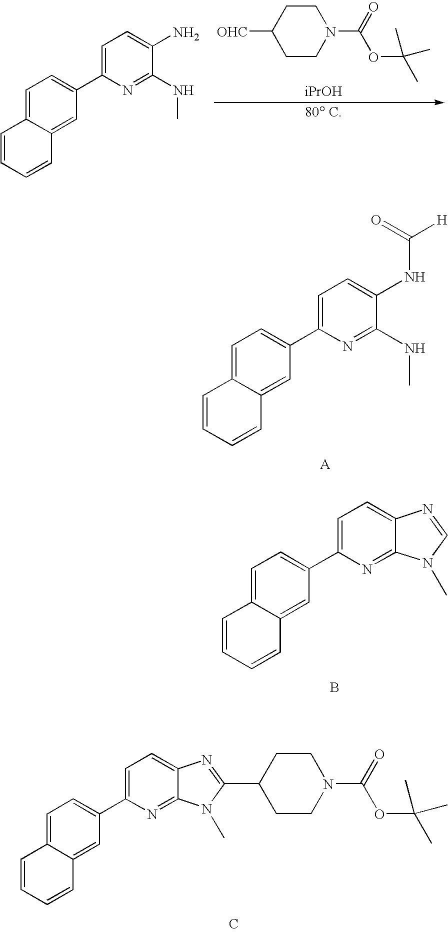

tert-butyl 4-[3-methyl-5-(2-naphthyl)-3H-imidazo[4,5-b]pyridin-2-yl]piperidine-1-carboxylate. A solution of N2-methyl-6-(naphthalen-2-yl)pyridine-2,3-diamine (40 mg, 0.16 mmol) in isopropanol (3 mL) was added to a round bottom flask containing tert-butyl 4-formylpiperidine-1-carboxylate (68.3 mg, 0.32 mmol) and heated at 80° C. open to ambient atmosphere with stirring over an oil bath for 16 h. The reaction was removed from the heat and allowed to cool to room temperature and concentrated under reduced pressure.
N-[2-(methylamino)-6-(2-naphthyl)pyridin-3-yl]formamide. Compound A was obtained during the purification as a white solid (13.5 mg, 30.4%). HPLC (method A): Rt=9.6 min. MS: [M+H]⁺=278, [M−H]⁻=276.
3-methyl-5-(2-naphthyl)-3H-imidazo[4,5-b]pyridine. Compound B was obtained during the purification as an off-white solid (7 mg, 16.9%). HPLC (method A): Rt=97 min. MS: [M+H]⁺=260.

### Example 62

### 4-[7-(2-Naphthyl)-3-piperidin-4-yl-3H-imidazo[4,5-b]pyridin-2-yl]phenol

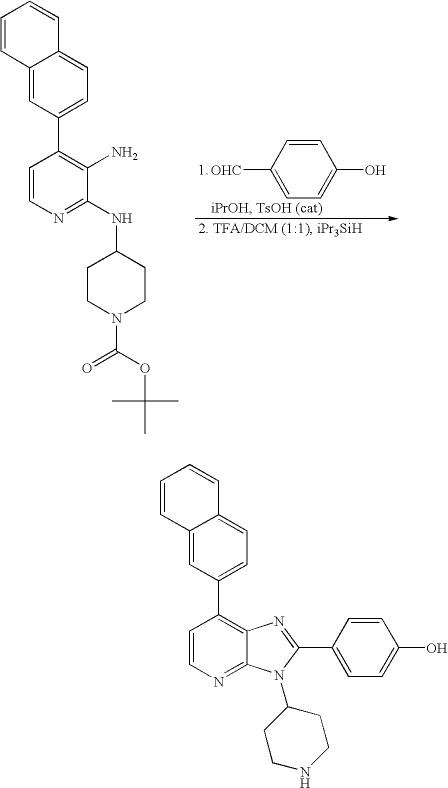

4-[7-(2-Naphthyl)-3-piperidin-4-yl-3H-imidazo[4,5-b]pyridin-2-yl]phenol. An aliquot of a stock solution of the diamine (2 mL of 10 mg/mL solution, 0.0478 mmol, 0.020 mg) in isopropanol was added to a glass vial containing 4-hydroxybenzaldehyde (6.4 mg, 0.0526 mmol) followed by p-toluenesulfonic acid monohydrate (2.3 mg, 0.0119 mmol). The vial was sealed with a teflon-lined cap. The mixture was heated at 80° C. in an Orbital shaker for 3 h, removed from the heat and allowed to cool while open to air. The lid was tightened, and the vial was heated at 80° C. overnight. The solvent was removed in a Genevac evaporator, and the residue was re-dissolved in a mixture of TFA/DCM (1:1) containing triisopropylsilane (15 μL, 0.0574 mmol). The mixture was shaken at room temperature for 30 to 60 min, and then concentrated to a residue using a Genevac evaporator. The crude residue was purified by semi-prep HPLC (Method D) to obtain the TFA salt of the product (21 mg, 58%) as an oily residue. Examples 63-155 in the following table were prepared with the similar procedures as in as Example 62.

| | | | R.t | HPLC |
|---|---|---|---|---|
| Example No. | Compound | MS | (mins.) | Method |
| 62 | 4-[7-(2-naphthyl)-3-piperidin-4-yl-3H- | 421 | 3.59 | C |
| | imidazo[4,5-b]pyridin-2-yl]phenol | | | |
| 63 | 3-[7-(2-naphthyl)-3-piperidin-4-yl-3H- | 421 | 3.60 | C |
| | imidazo[4,5-b]pyridin-2-yl]phenol | | | |
| 64 | 2-[7-(2-naphthyl)-3-piperidin-4-yl-3H- | 421 | 3.71 | C |
| | imidazo[4,5-b]pyridin-2-yl]phenol | | | |
| 65 | 3-[7-(2-naphthyl)-3-piperidin-4-yl-3H- | 437 | 3.53 | C |
| | imidazo[4,5-b]pyridin-2-yl]benzene-1,2-diol | | | |
| 66 | 2-methoxy-4-[7-(2-naphthyl)-3-piperidin-4-yl- | 451 | 3.60 | C |
| | 3H-imidazo[4,5-b]pyridin-2-yl]phenol | | | |
| 67 | 3-[7-(2-naphthyl)-3-piperidin-4-yl-3H- | 466 | 3.70 | C |
| | imidazo[4,5-b]pyridin-2-yl]-4-nitrophenol | | | |
| 68 | 2-(4-methoxyphenyl)-7-(2-naphthyl)-3- | 435 | 3.87 | C |
| | piperidin-4-yl-3H-imidazo[4,5-b]pyridine | | | |
| 69 | 2-(2-bromophenyl)-7-(2-naphthyl)-3-piperidin- | 483 | 3.96 | C |
| | 4-yl-3H-imidazo[4,5-b]pyridine | | | |
| 70 | 2-(3-bromophenyl)-7-(2-naphthyl)-3-piperidin- | 483 | 4.14 | C |
| | 4-yl-3H-imidazo[4,5-b]pyridine | | | |
| 71 | 2-(4-bromophenyl)-7-(2-naphthyl)-3-piperidin- | 483 | 4.12 | C |
| | 4-yl-3H-imidazo[4,5-b]pyridine | | | |
| 72 | 7-(2-naphthyl)-3-piperidin-4-yl-2-(3- | 431 | 4.10 | C |
| | vinylphenyl)-3H-imidazo[4,5-b]pyridine | | | |
| 73 | 4-[7-(2-naphthyl)-3-piperidin-4-yl-3H- | 572 | 4.92 | C |
| | imidazo[4,5-b]pyridin-2-yl]-N,N- | | | |
| | diphenylaniline | | | |
| 74 | 2-(2-methoxy-1-naphthyl)-7-(2-naphthyl)-3- | 485 | 4.13 | C |
| | piperidin-4-yl-3H-imidazo[4,5-b]pyridine | | | |
| 75 | 7-(2-naphthyl)-2-(3-phenoxyphenyl)-3- | 497 | 4.37 | C |
| | piperidin-4-yl-3H-imidazo[4,5-b]pyridine | | | |
| 76 | 7-(2-naphthyl)-3-piperidin-4-yl-2-{3-[3- | 565 | 4.61 | C |
| | (trifluoromethyl)phenoxy]phenyl}-3H- | | | |
| | imidazo[4,5-b]pyridine | | | |
| 77 | 7-(2-naphthyl)-3-piperidin-4-yl-2-[4- | 473 | 4.22 | C |
| | (trifluoromethyl)phenyl]-3H-imidazo[4,5- | | | |
| | b]pyridine | | | |
| 78 | 7-(2-naphthyl)-3-piperidin-4-yl-2-[3- | 473 | 4.21 | C |
| | (trifluoromethyl)phenyl]-3H-imidazo[4,5- | | | |
| | b]pyridine | | | |
| 79 | 7-(2-naphthyl)-3-piperidin-4-yl-2-[2- | 473 | 4.02 | C |
| | (trifluoromethyl)phenyl]-3H-imidazo[4,5- | | | |
| | b]pyridine | | | |
| 80 | 2-(2,6-difluorophenyl)-7-(2-naphthyl)-3- | 441 | 3.88 | C |
| | piperidin-4-yl-3H-imidazo[4,5-b]pyridine | | | |
| 81 | 2-(2,3-difluorophenyl)-7-(2-naphthyl)-3- | 441 | 3.99 | C |
| | piperidin-4-yl-3H-imidazo[4,5-b]pyridine | | | |
| 82 | 7-(2-naphthyl)-3-piperidin-4-yl-2-[4- | 489 | 4.29 | C |
| | (trifluoromethoxy)phenyl]-3H-imidazo[4,5- | | | |
| | b]pyridine | | | |
| 83 | 7-(2-naphthyl)-3-piperidin-4-yl-2-[2- | 489 | 4.09 | C |
| | (trifluoromethoxy)phenyl]-3H-imidazo[4,5- | | | |
| | b]pyridine | | | |
| 84 | 2-[2,5-bis(trifluoromethyl)phenyl]-7-(2- | 541 | 4.32 | C |
| | naphthyl)-3-piperidin-4-yl-3H-imidazo[4,5- | | | |
| | b]pyridine | | | |
| 85 | 2-[4-fluoro-2-(trifluoromethyl)phenyl]-7-(2- | 491 | 4.09 | C |
| | naphthyl)-3-piperidin-4-yl-3H-imidazo[4,5- | | | |
| | b]pyridine | | | |
| 86 | 2-[2-chloro-3-(trifluoromethyl)phenyl]-7-(2- | 507 | 4.25 | C |
| | naphthyl)-3-piperidin-4-yl-3H-imidazo[4,5- | | | |
| | b]pyridine | | | |
| 87 | 2-(3-fluorophenyl)-7-(2-naphthyl)-3-piperidin- | 423 | 3.94 | C |
| | 4-yl-3H-imidazo[4,5-b]pyridine | | | |
| 88 | 2-(2-chlorophenyl)-7-(2-naphthyl)-3-piperidin- | 439 | 3.93 | C |
| | 4-yl-3H-imidazo[4,5-b]pyridine | | | |
| 89 | 2-(4-fluorophenyl)-7-(2-naphthyl)-3-piperidin- | 423 | 3.94 | C |
| | 4-yl-3H-imidazo[4,5-b]pyridine | | | |
| 90 | 4-[7-(2-naphthyl)-3-piperidin-4-yl-3H- | 430 | 3.83 | C |
| | imidazo[4,5-b]pyridin-2-yl]benzonitrile | | | |
| 91 | N-{4-[7-(2-naphthyl)-3-piperidin-4-yl-3H- | 462 | 3.58 | C |
| | imidazo[4,5-b]pyridin-2-yl]phenyl}acetamide | | | |
| 92 | 7-(2-naphthyl)-2-(4-nitrophenyl)-3-piperidin-4- | 450 | 3.98 | C |
| | yl-3H-imidazo[4,5-b]pyridine | | | |
| 93 | 2-(4-chlorophenyl)-7-(2-naphthyl)-3-piperidin- | 439 | 4.09 | C |
| | 4-yl-3H-imidazo[4,5-b]pyridine | | | |
| 94 | 2-(1-ethyl-5-methyl-1H-imidazol-4-yl)-7-(2- | 437 | 3.64 | C |
| | naphthyl)-3-piperidin-4-yl-3H-imidazo[4,5- | | | |
| | b]pyridine | | | |
| 95 | 2-(1-methyl-1H-imidazol-2-yl)-7-(2-naphthyl)- | 409 | 3.64 | C |
| | 3-piperidin-4-yl-3H-imidazo[4,5-b]pyridine | | | |
| 96 | 2-(4-methyl-1H-imidazol-5-yl)-7-(2-naphthyl)- | 409 | 3.22 | C |
| | 3-piperidin-4-yl-3H-imidazo[4,5-b]pyridine | | | |
| 97 | 2-(1H-imidazol-2-yl)-7-(2-naphthyl)-3- | 395 | 3.51 | C |
| | piperidin-4-yl-3H-imidazo[4,5-b]pyridine | | | |
| 98 | 2-(2-methyl-1H-imidazol-4-yl)-7-(2-naphthyl)- | 409 | 2.91 | C |
| | 3-piperidin-4-yl-3H-imidazo[4,5-b]pyridine | | | |
| 99 | 7-(2-naphthyl)-2-(5-nitro-2-thienyl)-3- | 456 | 4.14 | C |
| | piperidin-4-yl-3H-imidazo[4,5-b]pyridine | | | |
| 100 | 7-(2-naphthyl)-3-piperidin-4-yl-2-(3-thienyl)- | 411 | 3.79 | C |
| | 3H-imidazo[4,5-b]pyridine | | | |
| 101 | 2-(1-benzothien-3-yl)-7-(2-naphthyl)-3- | 461 | 4.18 | C |
| | piperidin-4-yl-3H-imidazo[4,5-b]pyridine | | | |
| 102 | 2-(5-methoxy-1H-indol-3-yl)-7-(2-naphthyl)-3- | 474 | 3.88 | C |
| | piperidin-4-yl-3H-imidazo[4,5-b]pyridine | | | |
| 103 | 2-(5-methyl-1H-indol-3-yl)-7-(2-naphthyl)-3- | 458 | 4.07 | C |
| | piperidin-4-yl-3H-imidazo[4,5-b]pyridine | | | |
| 104 | 2-(1-acetyl-1H-indol-3-yl)-7-(2-naphthyl)-3- | 486 | 4.14 | C |
| | piperidin-4-yl-3H-imidazo[4,5-b]pyridine | | | |
| 105 | 2-(4,5-dimethyl-2-furyl)-7-(2-naphthyl)-3- | 423 | 4.09 | C |
| | piperidin-4-yl-3H-imidazo[4,5-b]pyridine | | | |
| 106 | 7-(2-naphthyl)-2-(1-oxidopyridin-4-yl)-3- | 422 | 3.22 | C |
| | piperidin-4-yl-3H-imidazo[4,5-b]pyridine | | | |
| 107 | 2-(3,5-dichloropyridin-4-yl)-7-(2-naphthyl)-3- | 474 | 3.89 | C |
| | piperidin-4-yl-3H-imidazo[4,5-b]pyridine | | | |
| 108 | 2-(1-methyl-1H-pyrrol-2-yl)-7-(2-naphthyl)-3- | 408 | 3.85 | C |
| | piperidin-4-yl-3H-imidazo[4,5-b]pyridine | | | |
| 109 | 5-[7-(2-naphthyl)-3-piperidin-4-yl-3H- | 438 | 3.14 | C |
| | imidazo[4,5-b]pyridin-2-yl]pyrimidine- | | | |
| | 2,4(1H,3H)-dione | | | |
| 110 | 2-(2,3-dihydro-1,4-benzodioxin-6-yl)-7-(2- | 449 | 3.86 | C |
| | naphthyl)-3-piperidin-4-yl-3H-imidazo[4,5- | | | |
| | b]pyridine | | | |
| 111 | 2-(2-chloro-4-methyl-1-phenyl-1H-pyrrol-3-yl)- | 463 | 3.87 | C |
| | 7-(2-naphthyl)-3-piperidin-4-yl-3H- | | | |
| | imidazo[4,5-b]pyridine | | | |
| 112 | 2-(2-chloro-4-methyl-1-phenyl-1H-pyrrol-3-yl)- | 519 | 4.20 | C |
| | 7-(2-naphthyl)-3-piperidin-4-yl-3H- | | | |
| | imidazo[4,5-b]pyridine | | | |
| 113 | 2-chloro-3-[7-(2-naphthyl)-3-piperidin-4-yl-3H- | 490 | 4.09 | C |
| | imidazo[4,5-b]pyridin-2-yl]quinoline | | | |
| 114 | 3-[7-(2-naphthyl)-3-piperidin-4-yl-3H- | 456 | 3.85 | C |
| | imidazo[4,5-b]pyridin-2-yl]quinoline | | | |
| 115 | 2-[7-(2-naphthyl)-3-piperidin-4-yl-3H- | 456 | 4.34 | C |
| | imidazo[4,5-b]pyridin-2-yl]quinoline | | | |
| 116 | 2-cyclopentyl-7-(2-naphthyl)-3-piperidin-4-yl- | 397 | 4.10 | C |
| | 3H-imidazo[4,5-b]pyridine | | | |
| 117 | 7-(2-naphthyl)-3-piperidin-4-yl-2-[3- | 489 | 4.26 | C |
| | (trifluoromethoxy)phenyl]-3H-imidazo[4,5- | | | |
| | b]pyridine | | | |
| 118 | 7-(2-naphthyl)-3-piperidin-4-yl-2-pyridin-2-yl- | 406 | 3.76 | C |
| | 3H-imidazo[4,5-b]pyridine | | | |
| 119 | 7-(2-naphthyl)-3-piperidin-4-yl-2-pyridin-3-yl- | 406 | 3.42 | C |
| | 3H-imidazo[4,5-b]pyridine | | | |
| 120 | 7-(2-naphthyl)-3-piperidin-4-yl-2-pyridin-4-yl- | 406 | 3.39 | C |
| | 3H-imidazo[4,5-b]pyridine | | | |
| 121 | 7-(2-naphthyl)-3-piperidin-4-yl-2-(1H-pyrrol-2- | 394 | 4.34 | C |
| | yl)-3H-imidazo[4,5-b]pyridine | | | |
| 122 | 2-(1-methyl-1H-indol-2-yl)-7-(2-naphthyl)-3- | 458 | 3.92 | C |
| | piperidin-4-yl-3H-imidazo[4,5-b]pyridine | | | |
| 123 | 7-(2-naphthyl)-3-piperidin-4-yl-2-(2-thienyl)- | 411 | 3.92 | C |
| | 3H-imidazo[4,5-b]pyridine | | | |
| 124 | 2-(4-morpholin-4-yl-3-nitrophenyl)-7-(2- | 535 | 3.76 | C |
| | naphthyl)-3-piperidin-4-yl-3H-imidazo[4,5- | | | |
| | b]pyridine | | | |
| 125 | 8-[7-(2-naphthyl)-3-piperidin-4-yl-3H- | 456 | 3.61 | C |
| | imidazo[4,5-b]pyridin-2-yl]quinoline | | | |
| 126 | 5-[7-(2-naphthyl)-3-piperidin-4-yl-3H- | 456 | 3.61 | C |
| | imidazo[4,5-b]pyridin-2-yl]quinoline | | | |
| 127 | 2-(1-benzofuran-5-yl)-7-(2-naphthyl)-3- | 445 | 4.07 | C |
| | piperidin-4-yl-3H-imidazo[4,5-b]pyridine | | | |
| 128 | 2-(5-methyl-2-furyl)-7-(2-naphthyl)-3- | 409 | 3.93 | C |
| | piperidin-4-yl-3H-imidazo[4,5-b]pyridine | | | |
| 129 | 2-(1-methyl-1H-indol-3-yl)-7-(2-naphthyl)-3- | 458 | 4.18 | C |
| | piperidin-4-yl-3H-imidazo[4,5-b]pyridine | | | |
| 130 | 2-(2-methoxypyridin-3-yl)-7-(2-naphthyl)-3- | 436 | 3.81 | C |
| | piperidin-4-yl-3H-imidazo[4,5-b]pyridine | | | |
| 131 | 4-[7-(2-naphthyl)-3-piperidin-4-yl-3H- | 456 | 3.77 | C |
| | imidazo[4,5-b]pyridin-2-yl]quinoline | | | |
| 132 | 2-(2-furyl)-7-(2-naphthyl)-3-piperidin-4-yl-3H- | 395 | 3.71 | C |
| | imidazo[4,5-b]pyridine | | | |
| 133 | 2-(1-benzofuran-2-yl)-7-(2-naphthyl)-3- | 445 | 4.17 | C |
| | piperidin-4-yl-3H-imidazo[4,5-b]pyridine | | | |
| 134 | 2-(1-benzothien-2-yl)-7-(2-naphthyl)-3- | 461 | 4.28 | C |
| | piperidin-4-yl-3H-imidazo[4,5-b]pyridine | | | |
| 135 | 2-isobutyl-7-(2-naphthyl)-3-piperidin-4-yl-3H- | 385 | 3.76 | C |
| | imidazo[4,5-b]pyridine | | | |
| 136 | 2-cyclohexyl-7-(2-naphthyl)-3-piperidin-4-yl- | 411 | 4.16 | C |
| | 3H-imidazo[4,5-b]pyridine | | | |
| 137 | 7-(2-naphthyl)-3-piperidin-4-yl-2-propyl-3H- | 371 | 3.59 | C |
| | imidazo[4,5-b]pyridine | | | |
| 138 | 2-butyl-7-(2-naphthyl)-3-piperidin-4-yl-3H- | 385 | 3.8 | C |
| | imidazo[4,5-b]pyridine | | | |
| 139 | 2-ethyl-7-(2-naphthyl)-3-piperidin-4-yl-3H- | 357 | 3.43 | C |
| | imidazo[4,5-b]pyridine | | | |
| 140 | 2-(4-isopropylphenyl)-7-(2-naphthyl)-3- | 447 | 4.28 | C |
| | piperidin-4-yl-3H-imidazo[4,5-b]pyridine | | | |
| 141 | 2-(4-tert-butylphenyl)-7-(2-naphthyl)-3- | 461 | 4.39 | C |
| | piperidin-4-yl-3H-imidazo[4,5-b]pyridine | | | |
| 142 | 2-(3-methoxyphenyl)-7-(2-naphthyl)-3- | 435 | 3.87 | C |
| | piperidin-4-yl-3H-imidazo[4,5-b]pyridine | | | |
| 143 | 7-(2-naphthyl)-2-(2-nitrophenyl)-3-piperidin-4- | 450 | 3.8 | C |
| | yl-3H-imidazo[4,5-b]pyridine | | | |
| 144 | 2-(4-methylphenyl)-7-(2-naphthyl)-3-piperidin- | 419 | 3.97 | C |
| | 4-yl-3H-imidazo[4,5-b]pyridine | | | |
| 145 | 7-(2-naphthyl)-2-(3-nitrophenyl)-3-piperidin-4- | 450 | 3.92 | C |
| | yl-3H-imidazo[4,5-b]pyridine | | | |
| 146 | 7-(2-naphthyl)-2-phenyl-3-piperidin-4-yl-3H- | 405 | 3.8 | C |
| | imidazo[4,5-b]pyridine | | | |
| 147 | 2-(2-methoxyphenyl)-7-(2-naphthyl)-3- | 435 | 3.81 | C |
| | piperidin-4-yl-3H-imidazo[4,5-b]pyridine | | | |
| 148 | 2-(3-methylphenyl)-7-(2-naphthyl)-3-piperidin- | 419 | 3.97 | C |
| | 4-yl-3H-imidazo[4,5-b]pyridine | | | |
| 149 | 2-(2,4-dimethylphenyl)-7-(2-naphthyl)-3- | 433 | 4.04 | C |
| | piperidin-4-yl-3H-imidazo[4,5-b]pyridine | | | |
| 150 | 2-(2,4-dichlorophenyl)-7-(2-naphthyl)-3- | 474 | 4.14 | C |
| | piperidin-4-yl-3H-imidazo[4,5-b]pyridine | | | |
| 151 | 2-(1-naphthyl)-7-(2-naphthyl)-3-piperidin-4-yl- | 455 | 4.06 | C |
| | 3H-imidazo[4,5-b]pyridine | | | |
| 152 | 2,7-di-2-naphthyl-3-piperidin-4-yl-3H- | 455 | 4.19 | C |
| | imidazo[4,5-b]pyridine | | | |
| 153 | 2-(2,4-difluorophenyl)-7-(2-naphthyl)-3- | 441 | 3.91 | C |
| | piperidin-4-yl-3H-imidazo[4,5-b]pyridine | | | |
| 154 | 2-(3-chlorophenyl)-7-(2-naphthyl)-3-piperidin- | 439 | 4.07 | C |
| | 4-yl-3H-imidazo[4,5-b]pyridine | | | |
| 155 | 2-(2-fluorophenyl)-7-(2-naphthyl)-3-piperidin- | 423 | 3.84 | C |
| | 4-yl-3H-imidazo[4,5-b]pyridine | | | |

### Example 156

### 4-[6-(2-naphthyl)-3-piperidin-4-yl-3H-imidazo[4,5-b]pyridin-2-yl]phenol

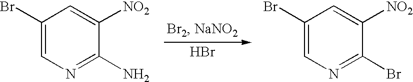

2,5-dibromo-3-nitropyridine. 2-Amino-5-bromo-3-nitropyridine (4.70 g, 21.5 mmol) was suspended in hydrobromic acid and cooled in an ice bath. Bromice (3.85 mL, 75.3 mmol) was carefully added dropwise, followed by sodium nitrite (4.45 g, 64.5 mmol) portionwise with stirring. After 45 min., the mixture was warmed to rt. An additional portion of sodium nitrite (10.0 g, 144.9 mmol) was added, followed by an additional 10 mL of hydrobromic acid and water (20 mL.) Stirring was continued at rt. until no additional effervescence was observed. The mixture was poured into an Erlenmeyer flask and carefully basified with aq. sodium carbonate followed by addition of excess aq. sodium thiosulfate. The mixture was extracted with dichloromethane (3×100 mL). The organic layers were washed with water, brine and 5% sodium thiosulfate, dried over magnesium sulfate, filtered and concentrated under reduced pressure to yield the title compound. 4.58 g (75%). HPLC (method A): Rt=8.6 min. MS: [M]⁺=280.

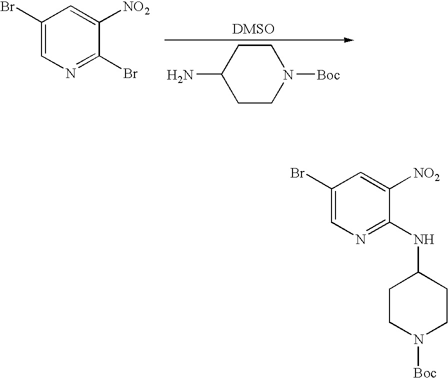

tert-Butyl 4-[(5-bromo-3-nitropyridin-2-yl)amino]piperidine-1-carboxylate. 2,5-Dibromo-3-nitropyridine (0.200 g, 0.71 mmol) was dissolved in DMSO (3 mL) and treated with 4-amino-1-boc-piperidine (0.428 g, 2.14 mmol.) The mixture was heated at 80° C. overnight and then diluted with ethyl acetate (30 mL.) The organic solution was washed with water (3×25 mL), saturated sodium bicarbonate (25 mL) and brine (25 mL). The organic layer was dried over magnesium sulfate, filtered and concentrated under reduced pressure. The crude material was purified by automated flash silica gel chromatography (ISCO Redi-Sep column) eluting with 0-100% ethyl acetate/hexane to yield the title compound. 0.229 g (80%). HPLC (method A): Rt=11.2 min. MS: [M+H-t-butyl]⁺=344.8.

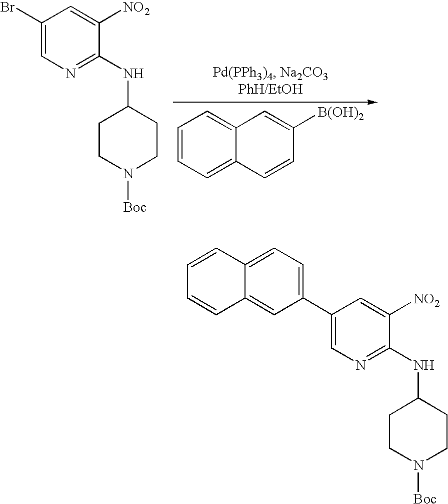

tert-Butyl 4-{[5-(2-naphthyl)-3-nitropyridin-2-yl]amino}piperidine-1-carboxylate. tert-Butyl 4-[(5-bromo-3-nitropyridin-2-yl)amino]piperidine-1-carboxylate (0.173 g, 0.43 mmol) and tetrakistriphenyl-phosphinepalladium(0) (0.015 g, 0.013 mmol) were combined in a flaskl and dissolved in degassed benzene. Separately, 2-naphthylboronic acid (0.081 g, 0.47 mmol) was dissolved in a minimum amount of degassed ethanol and treated with 0.5 mL of sodium carbonate solution (2.0 M, degassed.) The sodium carbonate mixture was added to the benzene solution. The flask was purged with nitrogen and heated to reflux for 1.5 h, at which time another portion (10 mg) of tetrakistriphenyl-phosphinepalladium(0) was added. After an additional 1.5 h, the mixture was diluted with ethyl acetate (25 mL) and washed with water and brine. The organic layer was dried over magnesium sulfate, filtered and concentrated under reduced pressure. The crude residue was purified by automated flash silica gel chromatography using ethyl acetate/hexane to yield the title compound. 0.107 g (55%). HPLC (method A): Rt=12.2 min. MS: [M+H]⁺=449.

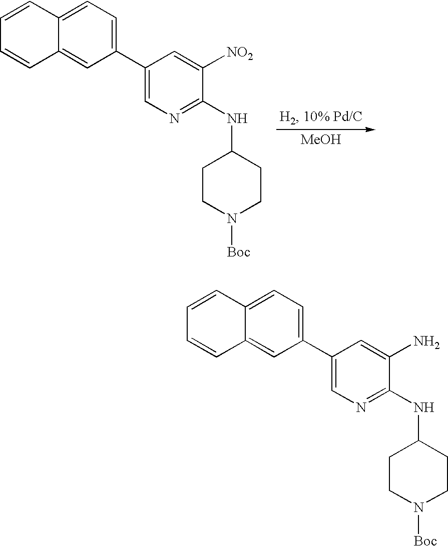

tert-Butyl 4-{[3-amino-5-(2-naphthyl)pyridin-2-yl]amino}piperidine-1-carboxylate. tert-Butyl 4-{[5-(2-naphthyl)-3-nitropyridin-2-yl]amino}piperidine-1-carboxylate (2.12 g, 4.73 mmol) was dissolved in methanol (60 mL) and added to 10% Pd/C (0.200 g) in a round-bottom flask. The flask was evacuated and refilled with hydrogen two times, and the reaction mixture was stirred under a balloon of hydrogen at rt overnight. The mixture was filtered through celite, and the celite was washed with methanol. The filtrate and washings were concentrated under reduced pressure to yield the title compound. 1.98 g (quant). HPLC (method E): Rt=10.9 min. MS: [M+H]⁺=419.3.

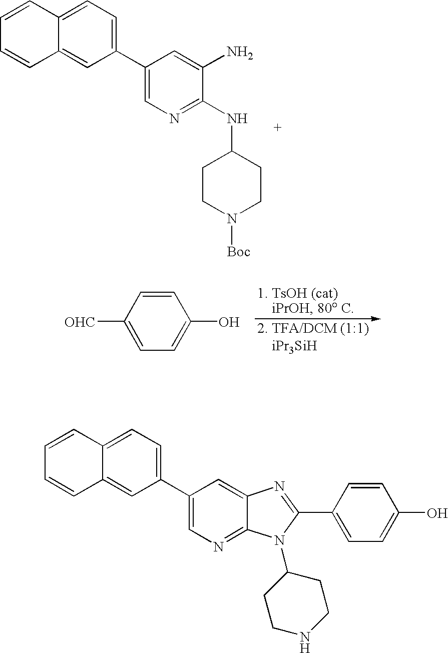

4-[6-(2-naphthyl)-3-piperidin-4-yl-3H-imidazo[4,5-b]pyridin-2-yl]phenol. An aliquot of a stock solution of tert-butyl 4-{[3-amino-5-(2-naphthyl)pyridin-2-yl]amino}piperidine-1-carboxylate (2 mL of 10 mg/mL solution, 0.0478 mmol, 0.020 mg) in isopropanol was added to a glass vial containing 4-hydroxybenzaldehyde (6.4 mg, 0.0526 mmol) followed by p-toluenesulfonic acid monohydrate (2.3 mg, 0.0119 mmol). The vial was sealed with a teflon-lined cap. The mixture was heated at 80° C. in an Orbital shaker for 3 h, removed from the heat and allowed to cool while open to air. The lid was tightened, and the vial was heated at 80° C. overnight. The solvent was removed in a Genevac evaporator, and the residue was re-dissolved in a mixture of TFA/DCM (1:1) containing triisopropylsilane (15 μL, 0.0574 mmol). The mixture was shaken at room temperature for 30 to 60 min, and then concentrated to a residue using a Genevac evaporator. The crude residue was purified by semi-prep HPLC (Method D) to obtain the TFA salt of the product (11.6 mg, 32%) as an oily residue. Examples 157-254 of the following table were prepared with the similar procedures as in Example 156.

| Example | | | R.t | HPLC |
|---|---|---|---|---|
| No. | Compound | MS | (mins.) | Method |
| 156 | 4-[6-(2-naphthyl)-3-piperidin-4-yl-3H-imidazo[4,5- | 421.2 | 1.93 | E |
| | b]pyridin-2-yl]phenol | | | |
| 157 | 3-[6-(2-naphthyl)-3-piperidin-4-yl-3H-imidazo[4,5- | 421.2 | 1.99 | E |
| | b]pyridin-2-yl]phenol | | | |
| 158 | 2-[6-(2-naphthyl)-3-piperidin-4-yl-3H-imidazo[4,5- | 421.2 | 1.97 | E |
| | b]pyridin-2-yl]phenol | | | |
| 159 | 3-[6-(2-naphthyl)-3-piperidin-4-yl-3H-imidazo[4,5- | 437.2 | 1.92 | E |
| | b]pyridin-2-yl]benzene-1,2-diol | | | |
| 160 | 2-methoxy-4-[6-(2-naphthyl)-3-piperidin-4-yl-3H- | 451.2 | 1.95 | E |
| | imidazo[4,5-b]pyridin-2-yl]phenol | | | |
| 161 | 3-[6-(2-naphthyl)-3-piperidin-4-yl-3H-imidazo[4,5- | 466.2 | 2 | E |
| | b]pyridin-2-yl]-4-nitrophenol | | | |
| 162 | 2-(4-methoxyphenyl)-6-(2-naphthyl)-3-piperidin-4-yl-3H- | 435.2 | 2.04 | E |
| | imidazo[4,5-b]pyridine | | | |
| 163 | 2-(2-bromophenyl)-6-(2-naphthyl)-3-piperidin-4-yl-3H- | 483.1 | 2.11 | E |
| | imidazo[4,5-b]pyridine | | | |
| 164 | 2-(3-bromophenyl)-6-(2-naphthyl)-3-piperidin-4-yl-3H- | 483.1 | 2.17 | E |
| | imidazo[4,5-b]pyridine | | | |
| 165 | 2-(4-bromophenyl)-6-(2-naphthyl)-3-piperidin-4-yl-3H- | 483.1 | 2.15 | E |
| | imidazo[4,5-b]pyridine | | | |
| 166 | 6-(2-naphthyl)-3-piperidin-4-yl-2-(3-vinylphenyl)-3H- | 431.2 | 2.15 | E |
| | imidazo[4,5-b]pyridine | | | |
| 167 | 4-[6-(2-naphthyl)-3-piperidin-4-yl-3H-imidazo[4,5- | 572.3 | 2.42 | E |
| | b]pyridin-2-yl]-N,N-diphenylaniline | | | |
| 168 | 2-(2-methoxy-1-naphthyl)-6-(2-naphthyl)-3-piperidin-4-yl- | 485.2 | 2.15 | E |
| | 3H-imidazo[4,5-b]pyridine | | | |
| 169 | 6-(2-naphthyl)-2-(3-phenoxyphenyl)-3-piperidin-4-yl-3H- | 497.2 | 2.26 | E |
| | imidazo[4,5-b]pyridine | | | |
| 170 | 6-(2-naphthyl)-3-piperidin-4-yl-2-{3-[3- | 565.2 | 2.38 | E |
| | (trifluoromethyl)phenoxy]phenyl}-3H-imidazo[4,5- | | | |
| | b]pyridine | | | |
| 171 | 6-(2-naphthyl)-3-piperidin-4-yl-2-[4- | 473.2 | 2.18 | E |
| | (trifluoromethyl)phenyl]-3H-imidazo[4,5-b]pyridine | | | |
| 172 | 6-(2-naphthyl)-3-piperidin-4-yl-2-[3- | 473.2 | 2.19 | E |
| | (trifluoromethyl)phenyl]-3H-imidazo[4,5-b]pyridine | | | |
| 173 | 6-(2-naphthyl)-3-piperidin-4-yl-2-[2- | 473.2 | 2.14 | E |
| | (trifluoromethyl)phenyl]-3H-imidazo[4,5-b]pyridine | | | |
| 174 | 2-(2,6-difluorophenyl)-6-(2-naphthyl)-3-piperidin-4-yl-3H- | 441.2 | 2.09 | E |
| | imidazo[4,5-b]pyridine | | | |
| 175 | 2-(2,3-difluorophenyl)-6-(2-naphthyl)-3-piperidin-4-yl-3H- | 441.2 | 2.11 | E |
| | imidazo[4,5-b]pyridine | | | |
| 176 | 6-(2-naphthyl)-3-piperidin-4-yl-2-[4- | 489.2 | 2.2 | E |
| | (trifluoromethoxy)phenyl]-3H-imidazo[4,5-b]pyridine | | | |
| 177 | 6-(2-naphthyl)-3-piperidin-4-yl-2-[2- | 489.2 | 2.18 | E |
| | (trifluoromethoxy)phenyl]-3H-imidazo[4,5-b]pyridine | | | |
| 178 | 2-[2,5-bis(trifluoromethyl)phenyl]-6-(2-naphthyl)-3- | 541.2 | 2.25 | E |
| | piperidin-4-yl-3H-imidazo[4,5-b]pyridine | | | |
| 179 | 2-[4-fluoro-2-(trifluoromethyl)phenyl]-6-(2-naphthyl)-3- | 491.2 | 3.97 | H |
| | piperidin-4-yl-3H-imidazo[4,5-b]pyridine | | | |
| 180 | 2-[2-chloro-3-(trifluoromethyl)phenyl]-6-(2-naphthyl)-3- | 507.1 | 2.22 | E |
| | piperidin-4-yl-3H-imidazo[4,5-b]pyridine | | | |
| 181 | 2-(3-fluorophenyl)-6-(2-naphthyl)-3-piperidin-4-yl-3H- | 423.2 | 2.08 | E |
| | imidazo[4,5-b]pyridine | | | |
| 182 | 2-(2-chlorophenyl)-6-(2-naphthyl)-3-piperidin-4-yl-3H- | 439.2 | 2.09 | E |
| | imidazo[4,5-b]pyridine | | | |
| 183 | 2-(4-fluorophenyl)-6-(2-naphthyl)-3-piperidin-4-yl-3H- | 423.2 | 2.05 | E |
| | imidazo[4,5-b]pyridine | | | |
| 184 | 4-[6-(2-naphthyl)-3-piperidin-4-yl-3H-imidazo[4,5- | 430.2 | 2.04 | E |
| | b]pyridin-2-yl]benzonitrile | | | |
| 185 | N-{4-[6-(2-naphthyl)-3-piperidin-4-yl-3H-imidazo[4,5- | 462.2 | 1.98 | E |
| | b]pyridin-2-yl]phenyl}acetamide | | | |
| 186 | 6-(2-naphthyl)-2-(4-nitrophenyl)-3-piperidin-4-yl-3H- | 450.2 | 2.09 | E |
| | imidazo[4,5-b]pyridine | | | |
| 187 | 2-(4-chlorophenyl)-6-(2-naphthyl)-3-piperidin-4-yl-3H- | 439.2 | 2.12 | E |
| | imidazo[4,5-b]pyridine | | | |
| 188 | 2-(1-ethyl-5-methyl-1H-imidazol-4-yl)-6-(2-naphthyl)-3- | 437.2 | 2.02 | E |
| | piperidin-4-yl-3H-imidazo[4,5-b]pyridine | | | |
| 189 | 2-(1-methyl-1H-imidazol-2-yl)-6-(2-naphthyl)-3-piperidin-4- | 409.2 | 3.52 | H |
| | yl-3H-imidazo[4,5-b]pyridine | | | |
| 190 | 2-(4-methyl-1H-imidazol-5-yl)-6-(2-naphthyl)-3-piperidin-4- | 409.2 | 1.86 | E |
| | yl-3H-imidazo[4,5-b]pyridine | | | |
| 191 | 2-(1H-imidazol-2-yl)-6-(2-naphthyl)-3-piperidin-4-yl-3H- | 395.2 | 1.96 | E |
| | imidazo[4,5-b]pyridine | | | |
| 192 | 2-(2-methyl-1H-imidazol-4-yl)-6-(2-naphthyl)-3-piperidin-4- | 409.2 | 1.76 | E |
| | yl-3H-imidazo[4,5-b]pyridine | | | |
| 193 | 6-(2-naphthyl)-2-(5-nitro-2-thienyl)-3-piperidin-4-yl-3H- | 456.1 | 2.22 | E |
| | imidazo[4,5-b]pyridine | | | |
| 194 | 6-(2-naphthyl)-3-piperidin-4-yl-2-(3-thienyl)-3H- | 411.2 | 2.02 | E |
| | imidazo[4,5-b]pyridine | | | |
| 195 | 2-(1-benzothien-3-yl)-6-(2-naphthyl)-3-piperidin-4-yl-3H- | 461.2 | 2.17 | E |
| | imidazo[4,5-b]pyridine | | | |
| 196 | 2-(5-methoxy-1H-indol-3-yl)-6-(2-naphthyl)-3-piperidin-4- | 474.2 | 1.99 | E |
| | yl-3H-imidazo[4,5-b]pyridine | | | |
| 197 | 2-(5-methyl-1H-indol-3-yl)-6-(2-naphthyl)-3-piperidin-4-yl- | 458.2 | 2.06 | E |
| | 3H-imidazo[4,5-b]pyridine | | | |
| 198 | 2-(1-acetyl-1H-indol-3-yl)-6-(2-naphthyl)-3-piperidin-4-yl- | 486.2 | 2.19 | E |
| | 3H-imidazo[4,5-b]pyridine | | | |
| 199 | 2-(3-furyl)-6-(2-naphthyl)-3-piperidin-4-yl-3H-imidazo[4,5- | 395.2 | 1.99 | E |
| | b]pyridine | | | |
| 200 | 2-(4,5-dimethyl-2-furyl)-6-(2-naphthyl)-3-piperidin-4-yl-3H- | 423.2 | 2.18 | E |
| | imidazo[4,5-b]pyridine | | | |
| 201 | 6-(2-naphthyl)-2-(1-oxidopyridin-4-yl)-3-piperidin-4-yl-3H- | 422.2 | 1.93 | E |
| | imidazo[4,5-b]pyridine | | | |
| 202 | 2-(3,5-dichloropyridin-4-yl)-6-(2-naphthyl)-3-piperidin-4-yl- | 474.1 | 2.11 | E |
| | 3H-imidazo[4,5-b]pyridine | | | |
| 203 | 2-(1-methyl-1H-pyrrol-2-yl)-6-(2-naphthyl)-3-piperidin-4-yl- | 408.2 | 3.72 | H |
| | 3H-imidazo[4,5-b]pyridine | | | |
| 204 | 5-[6-(2-naphthyl)-3-piperidin-4-yl-3H-imidazo[4,5- | 439.2 | 1.85 | E |
| | b]pyridin-2-yl]pyrimidine-2,4(1H,3H)-dione | | | |
| 205 | 2-(1,3-benzodioxol-5-yl)-6-(2-naphthyl)-3-piperidin-4-yl-3H- | 449.2 | 2.05 | E |
| | imidazo[4,5-b]pyridine | | | |
| 206 | 2-(2,3-dihydro-1,4-benzodioxin-6-yl)-6-(2-naphthyl)-3- | 463.2 | 2.06 | E |
| | piperidin-4-yl-3H-imidazo[4,5-b]pyridine | | | |
| 207 | 2-(2-chloro-4-methyl-1-phenyl-1H-pyrrol-3-yl)-6-(2- | 519.5 | 2.2 | E |
| | naphthyl)-3-piperidin-4-yl-3H-imidazo[4,5-b]pyridine | | | |
| 208 | 2-chloro-3-[6-(2-naphthyl)-3-piperidin-4-yl-3H-imidazo[4,5- | 490.2 | 2.15 | E |
| | b]pyridin-2-yl]quinoline | | | |
| 209 | 3-[6-(2-naphthyl)-3-piperidin-4-yl-3H-imidazo[4,5- | 456.2 | 2.11 | E |
| | b]pyridin-2-yl]quinoline | | | |
| 210 | 2-[6-(2-naphthyl)-3-piperidin-4-yl-3H-imidazo[4,5- | 456.2 | 2.26 | E |
| | b]pyridin-2-yl]quinoline | | | |
| 211 | 6-(2-naphthyl)-3-piperidin-4-yl-2-[3- | 489.2 | 2.21 | E |
| | (trifluoromethoxy)phenyl]-3H-imidazo[4,5-b]pyridine | | | |
| 212 | 2-(3-ethylphenyl)-6-(2-naphthyl)-3-piperidin-4-yl-3H- | 433.2 | 2.14 | E |
| | imidazo[4,5-b]pyridine | | | |
| 213 | 6-(2-naphthyl)-3-piperidin-4-yl-2-pyridin-2-yl-3H- | 406 | 3.7 | E |
| | imidazo[4,5-b]pyridine | | | |
| 214 | 6-(2-naphthyl)-3-piperidin-4-yl-2-pyridin-3-yl-3H- | 406 | 3.9 | E |
| | imidazo[4,5-b]pyridine | | | |
| 215 | 6-(2-naphthyl)-3-piperidin-4-yl-2-pyridin-4-yl-3H- | 406 | 2.7 | E |
| | imidazo[4,5-b]pyridine | | | |
| 216 | 6-(2-naphthyl)-3-piperidin-4-yl-2-(1H-pyrrol-2-yl)-3H- | | | E |
| | imidazo[4,5-b]pyridine | | | |
| 217 | 2-(1-methyl-1H-indol-2-yl)-6-(2-naphthyl)-3-piperidin-4-yl- | 458 | 3.8 | E |
| | 3H-imidazo[4,5-b]pyridine | | | |
| 218 | 6-(2-naphthyl)-3-piperidin-4-yl-2-(2-thienyl)-3H- | 411 | 3.5 | E |
| | imidazo[4,5-b]pyridine | | | |
| 219 | 2-(4-morpholin-4-yl-3-nitrophenyl)-6-(2-naphthyl)-3- | 535 | 3.5 | E |
| | piperidin-4-yl-3H-imidazo[4,5-b]pyridine | | | |
| 220 | 8-[6-(2-naphthyl)-3-piperidin-4-yl-3H-imidazo[4,5- | 456 | 3.4 | E |
| | b]pyridin-2-yl]quinoline | | | |
| 221 | 5-[6-(2-naphthyl)-3-piperidin-4-yl-3H-imidazo[4,5- | 456 | 2.8 | E |
| | b]pyridin-2-yl]quinoline | | | |
| 222 | 2-(1H-indol-3-yl)-6-(2-naphthyl)-3-piperidin-4-yl-3H- | 444 | 3.4 | E |
| | imidazo[4,5-b]pyridine | | | |
| 223 | 2-(1-benzofuran-5-yl)-6-(2-naphthyl)-3-piperidin-4-yl-3H- | 445 | 3.4 | E |
| | imidazo[4,5-b]pyridine | | | |
| 224 | 2-(5-methyl-2-furyl)-6-(2-naphthyl)-3-piperidin-4-yl-3H- | 409 | 3.5 | E |
| | imidazo[4,5-b]pyridine | | | |
| 225 | 2-(1-methyl-1H-indol-3-yl)-6-(2-naphthyl)-3-piperidin-4-yl- | | | E |
| | 3H-imidazo[4,5-b]pyridine | | | |
| 226 | 2-(2-methoxypyridin-3-yl)-6-(2-naphthyl)-3-piperidin-4-yl- | 436 | 3.3 | E |
| | 3H-imidazo[4,5-b]pyridine | | | |
| 227 | 4-[6-(2-naphthyl)-3-piperidin-4-yl-3H-imidazo[4,5- | 456 | 2.8 | E |
| | b]pyridin-2-yl]quinoline | | | |
| 228 | 2-(2-furyl)-6-(2-naphthyl)-3-piperidin-4-yl-3H-imidazo[4,5- | 395 | 3.4 | E |
| | b]pyridine | | | |
| 229 | 2-(1-benzofuran-2-yl)-6-(2-naphthyl)-3-piperidin-4-yl-3H- | 445 | 3.9 | E |
| | imidazo[4,5-b]pyridine | | | |
| 230 | 2-(1-benzothien-2-yl)-6-(2-naphthyl)-3-piperidin-4-yl-3H- | 461 | 4.1 | E |
| | imidazo[4,5-b]pyridine | | | |
| 231 | 2-isobutyl-6-(2-naphthyl)-3-piperidin-4-yl-3H-imidazo[4,5- | 385 | 3.6 | E |
| | b]pyridine | | | |
| 232 | 2-cyclohexyl-6-(2-naphthyl)-3-piperidin-4-yl-3H- | 411 | 3.6 | E |
| | imidazo[4,5-b]pyridine | | | |
| 233 | 6-(2-naphthyl)-3-piperidin-4-yl-2-propyl-3H-imidazo[4,5- | 371 | 3.6 | E |
| | b]pyridine | | | |
| 234 | 2-butyl-6-(2-naphthyl)-3-piperidin-4-yl-3H-imidazo[4,5- | 385 | 3.7 | E |
| | b]pyridine | | | |
| 235 | 2-(4-isopropylphenyl)-6-(2-naphthyl)-3-piperidin-4-yl-3H- | 447 | 3.8 | E |
| | imidazo[4,5-b]pyridine | | | |
| 236 | 2-(4-tert-butylphenyl)-6-(2-naphthyl)-3-piperidin-4-yl-3H- | 461 | 4 | E |
| | imidazo[4,5-b]pyridine | | | |
| 237 | 2-(3-methoxyphenyl)-6-(2-naphthyl)-3-piperidin-4-yl-3H- | 435 | 3.4 | E |
| | imidazo[4,5-b]pyridine | | | |
| 238 | 6-(2-naphthyl)-2-(2-nitrophenyl)-3-piperidin-4-yl-3H- | 450 | 3.4 | E |
| | imidazo[4,5-b]pyridine | | | |
| 239 | 2-(4-methylphenyl)-6-(2-naphthyl)-3-piperidin-4-yl-3H- | 419 | 3.5 | E |
| | imidazo[4,5-b]pyridine | | | |
| 240 | 6-(2-naphthyl)-2-(3-nitrophenyl)-3-piperidin-4-yl-3H- | 450 | 3.4 | E |
| | imidazo[4,5-b]pyridine | | | |
| 241 | 6-(2-naphthyl)-2-phenyl-3-piperidin-4-yl-3H-imidazo[4,5- | 405 | 3.4 | E |
| | b]pyridine | | | |
| 242 | 2-(2-methoxyphenyl)-6-(2-naphthyl)-3-piperidin-4-yl-3H- | 435 | 3.4 | E |
| | imidazo[4,5-b]pyridine | | | |
| 243 | 2-(3-methylphenyl)-6-(2-naphthyl)-3-piperidin-4-yl-3H- | 419 | 3.6 | E |
| | imidazo[4,5-b]pyridine | | | |
| 244 | 2-(2,4-dimethylphenyl)-6-(2-naphthyl)-3-piperidin-4-yl-3H- | 433 | 3.6 | E |
| | imidazo[4,5-b]pyridine | | | |
| 245 | 2-(2,4-dichlorophenyl)-6-(2-naphthyl)-3-piperidin-4-yl-3H- | 473 | 3.7 | E |
| | imidazo[4,5-b]pyridine | | | |
| 246 | 2-(1-naphthyl)-6-(2-naphthyl)-3-piperidin-4-yl-3H- | 455 | 3.6 | E |
| | imidazo[4,5-b]pyridine | | | |
| 247 | 2,6-di-2-naphthyl-3-piperidin-4-yl-3H-imidazo[4,5- | 455 | 3.7 | E |
| | b]pyridine | | | |
| 248 | 2-(2,4-difluorophenyl)-6-(2-naphthyl)-3-piperidin-4-yl-3H- | 441 | 3.4 | E |
| | imidazo[4,5-b]pyridine | | | |
| 249 | 2-(3-chlorophenyl)-6-(2-naphthyl)-3-piperidin-4-yl-3H- | 439 | 3.7 | E |
| | imidazo[4,5-b]pyridine | | | |
| 250 | 2-(2-fluorophenyl)-6-(2-naphthyl)-3-piperidin-4-yl-3H- | 423 | 3.5 | E |
| | imidazo[4,5-b]pyridine | | | |
| 251 | 6-(2-naphthyl)-3-piperidin-4-yl-3H-imidazo[4,5-b]pyridine | | | E |
| 252 | 2-methyl-6-(2-naphthyl)-3-piperidin-4-yl-3H-imidazo[4,5- | 343.2 | 1.92 | E |
| | b]pyridine | | | |
| 253 | 2-ethyl-6-(2-naphthyl)-3-piperidin-4-yl-3H-imidazo[4,5- | 357.2 | 1.92 | E |
| | b]pyridine | | | |
| 254 | 2-isopropyl-6-(2-naphthyl)-3-piperidin-4-yl-3H-imidazo[4,5- | 371.2 | 1.94 | E |
| | b]pyridine | | | |

### Example 255

### 7-(2-naphthyl)-3-piperidin-4-yl-1,3-dihydro-2H-imidazo[4,5-b]pyridine-2-thione

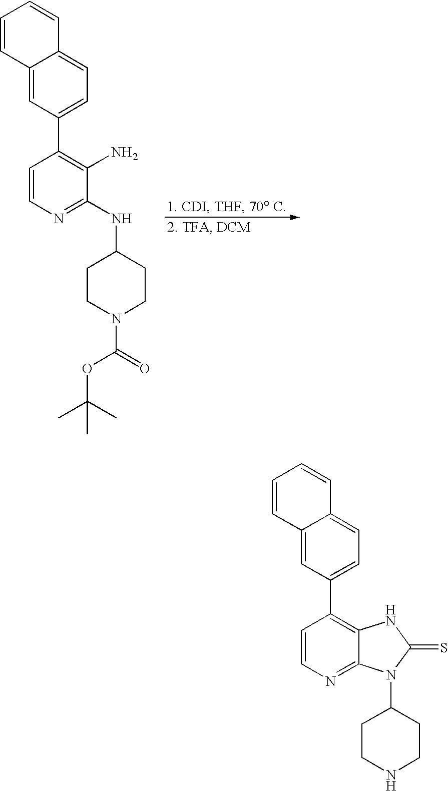

7-(2-naphthyl)-3-piperidin-4-yl-1,3-dihydro-2H-imidazo[4,5-b]pyridine-2-thione (WYE-101980): A solution of the phenylenediamine above (100 mg, 0.24 mmol) and 1,1′-thiocarbonyldiimidazole (85 mg, 0.48 mmol) in THF (2 mL) was stirred and heated to 70° C. for 2 hours. After cooling to room temperature the mixture was treated with water (0.5 mL) and purified directly by reversed phase HPLC (method D). The product fractions were evaporated and the residue was treated with TFA (1 mL) and methylene chloride (1 mL) for an hour. The solvents were evaporated to leave the product as a foamy solid. HPLC (method A) Rt=8.4 mins., purity=98.0%. HRMS ES [M+H]⁺=361.1479.

### Example 256

### tert-butyl 4-[7-(2-naphthyl)-2-oxo-1,2-dihydro-3H-imidazo[4,5-b]pyridin-3-yl]piperidine-1-carboxylate

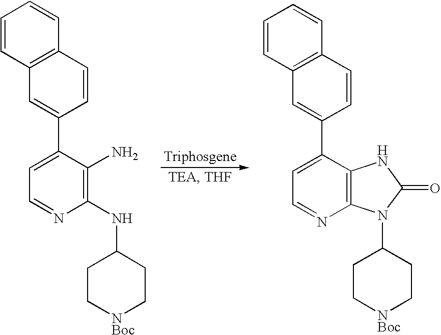

tert-butyl 4-[7-(2-naphthyl)-2-oxo-1,2-dihydro-3H-imidazo[4,5-b]pyridin-3-yl]piperidine-1-carboxylate. To a solution of the phenylenediamine above (50 mg, 0.120 mmol) in THF (1 mL) was added triethylamine (0.042 mL, 0.3 mmol) and triphosgene (14.2 mg, 0.048 mmol). The reaction was stirred at room temperature for 3 h. A solution of saturated sodium bicarbonate (1 mL) was added, the layers were separated, and the organic layer was concentrated to dryness. Recrystallized the product from methanol to yield the desired product. HPLC (method A) Rt=11.2 mins., purity=99.1%. HRMS ES [M+H]⁺=445.2218.

### Example 257

### tert-butyl 4-[1-methyl-7-(2-naphthyl)-2-oxo-1,2-dihydro-3H-imidazo[4,5-b]pyridin-3-yl]piperidine-1-carboxylate

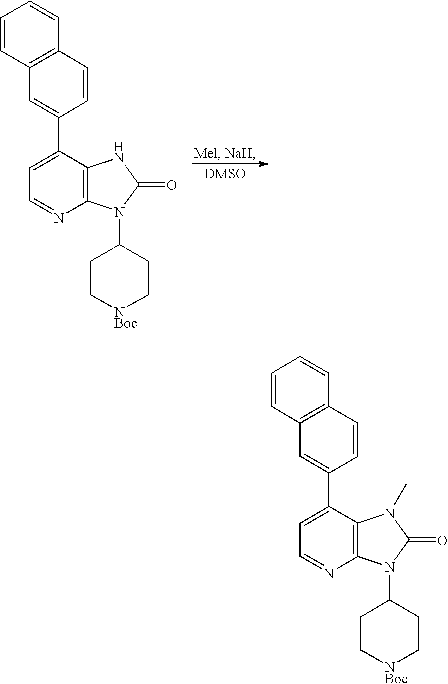

tert-butyl 4-[1-methyl-7-(2-naphthyl)-2-oxo-1,2-dihydro-3H-imidazo[4,5-b]pyridin-3-yl]piperidine-1-carboxylate. The urea above (350 mg, 0.788 mmol) was dissolved in DMSO (60 mL) with heating. The solution was cooled to room temperature and sodium hydride (49 mg, 1.22 mmol) was added. After 15 min of stirring, iodomethane (0.190 mL, 3.04 mmol) was added and the reaction was stirred at room temperature for 1 h. Water and ethyl acetate were added to the reaction. The aqueous layer was extracted with ethyl acetate (1×), and the combined organic extracts were washed with brine, dried with magnesium sulfate, concentrated and purified using silica gel chromatography (20%-50% ethyl acetate/hexane gradient) to yield the pure product. HPLC (method A) Rt=11.4 mins., purity=100.0%. HRMS ES [M+H-tbutyl]⁺=403.163

### Example 258

### 7-(2-naphthyl)-3-piperidin-4-yl-1,3-dihydro-2H-imidazo[4,5-b]pyridin-2-one

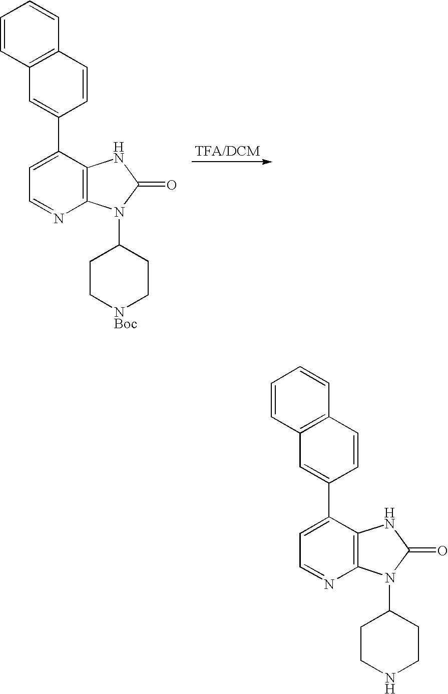

7-(2-naphthyl)-3-piperidin-4-yl-1,3-dihydro-2H-imidazo[4,5-b]pyridin-2-one. To a solution of the boc-protected piperidine (195 mg, 0.439 mmol.) in DCM (10 mL) was added TFA (10 mL). After 1 h, the reaction was concentrated to dryness. Toluene was added, and the mixture was concentrated to dryness. Repeated the toluene wash (2×) to yield the pure piperidine. HPLC (method A) Rt=7.7 mins., purity=99.6%. MS ES [M+H]⁺=344.9. Example 259 was prepared with the similar procedures as in Example 258.

| Example | | | R.t | HPLC |
|---|---|---|---|---|
| no. | Compound | MS | (mins.) | Method |
| 258 | 7-(2-naphthyl)-3-piperidin-4-yl-1,3-dihydro-2H- | 345 | 7.7 | A |
| | imidazo[4,5-b]pyridin-2-one | | | |
| 259 | 1-methyl-7-(2-naphthyl)-3-piperidin-4-yl-1,3-dihydro-2H- | 359 | 8.1 | A |
| | imidazo[4,5-b]pyridin-2-one | | | |

### Example 260

### N-isopropyl-4-[7-(2-naphthyl)-2-oxo-1,2-dihydro-3H-imidazo[4,5-b]pyridin-3-yl]piperidine-1-carboxamide

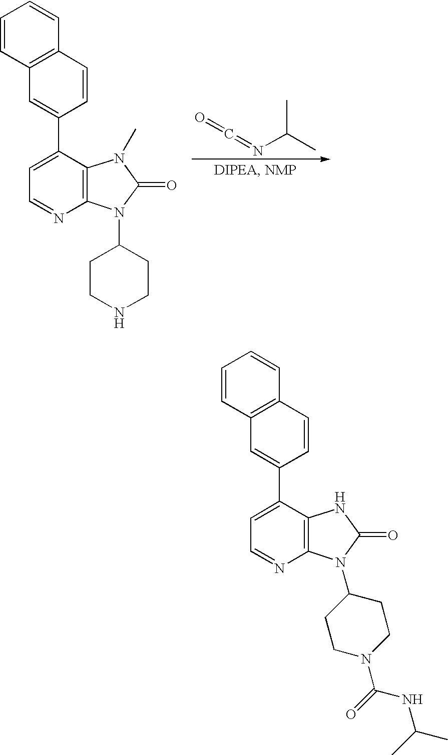

N-isopropyl-4-[7-(2-naphthyl)-2-oxo-1,2-dihydro-3H-imidazo[4,5-b]pyridin-3-yl]piperidine-1-carboxamide. To a solution of the piperidine above (20.0 mg, 0.035 mmol) in NMP (1 mL) was added diisopropylethylamine (0.019 mL, 0.105 mmol) and isopropylisocyanate (0.014 mL, 0.140 mmol). The reaction was stirred at room temperature for 18 h. Ethyl acetate and water were added and the aqueous layer was extracted with ethyl acetate (1×). The combined organic extracts were washed with water (2×) and brine (2×), dried with magnesium sulfate, concentrated and recrystallized from methanol to yield pure product. HPLC (open access) Rt=1.54 mins, MS ES [M+H]⁺=430.1. Examples 261-266 were prepared with the above similar procedures.

| Example | | | R.t | HPLC |
|---|---|---|---|---|
| no. | Compound | MS | (mins.) | Method |
| 260 | N-isopropyl-4-[7-(2-naphthyl)-2-oxo-1,2-dihydro-3H- | 430 | 1.54 | E |
| | imidazo[4,5-b]pyridin-3-yl]piperidine-1-carboxamide | | | |
| 261 | 3-[1-(morpholin-4-ylcarbonyl)piperidin-4-yl]-7-(2-naphthyl)- | 458 | 1.48 | E |
| | 1,3-dihydro-2H-imidazo[4,5-b]pyridin-2-one | | | |
| 262 | 3-(1-acetylpiperidin-4-yl)-1-methyl-7-(2-naphthyl)-1,3- | 401 | 1.57 | E |
| | dihydro-2H-imidazo[4,5-b]pyridin-2-one | | | |
| 263 | N-isopropyl-4-[1-methyl-7-(2-naphthyl)-2-oxo-1,2-dihydro- | 444 | 1.67 | E |
| | 3H-imidazo[4,5-b]pyridin-3-yl]piperidine-1-carboxamide | | | |
| 264 | N,N-dimethyl-4-[1-methyl-7-(2-naphthyl)-2-oxo-1,2- | 430 | 1.69 | E |
| | dihydro-3H-imidazo[4,5-b]pyridin-3-yl]piperidine-1- | | | |
| | carboxamide | | | |
| 265 | 1-methyl-3-[1-(morpholin-4-ylcarbonyl)piperidin-4-yl]-7-(2- | 472 | 1.63 | E |
| | naphthyl)-1,3-dihydro-2H-imidazo[4,5-b]pyridin-2-one | | | |
| 266 | 1-methyl-3-[1-(methylsulfonyl)piperidin-4-yl]-7-(2- | 437 | 1.71 | E |
| | naphthyl)-1,3-dihydro-2H-imidazo[4,5-b]pyridin-2-one | | | |

### Biological Evaluation—Functional Dkk1-LRP5-TCF-Luciferase Assay in U2OS Cells

U2OS Human Bone derived cells (Osteosarcoma) are grown in McCoy's 5A Medium (Modified), with L-glutamine (GIBCO Cat No. 16600-082)+1% Pen-Strep+5% FBS) plated at 1×107 cells/T175 cm flask. The next day, the cells are co-transfected overnight with the following plasmids: (a) Test reporter (16×TCF-TK-FireFly-Luci), (b) Internal Control Reporter (TK-Renilla-Luci), (c) Wnt3a and (d) Dkk1. GIBCO's Lipofectamine 2000 and OptiMEM were used for the transfection. After a minimum of 4 hr of transfection at 37° C., the plasmid-transfected cells are trypsinized, counted, and suspended in freezing medium (95% FBS+5% DMSO). The reporter cells are frozen at 1×107/ml concentrations, aliquoted into 0.5 ml or 2.5 ml/tube and stored at −70° C.

The following day, test compounds are added under HTS setup by Plate Track into 384 well plates (white, TC treated, Falcon plate) such that the final concentration of the compounds in 20 μL/well cell will be 5 μg/ml (final concentration of DMSO=0.25% and final compound concentration=20 μM). Vials of frozen reporter cells are thawed by warming the vial in a 37° C. water bath for 60-120 seconds with some shaking until the cells formed a suspension. The thawed cells are transferred into a cold 50 ml (or larger) tube and mixed well by gentle pipetting. The appropriate amount of cold Phenol Red Free RPMI medium-1640 (GIBCO, Cat # 11835-030) with L-glutamine is added, both with ˜5% FBS (GIBCO-BRL, Cat. # 16000-044), so that 20 μl of the final cell suspension will contain ˜5,000 cells. The cell dilution is done such that the final concentration of FBS was ˜5%. Diluted cells (20 μl) are added into each well in a 384 well plate. The plate is incubated at 37° C. under 5% CO₂ for ˜20 h. Bright-Glo substrate, 2.5 μl/well is added, and the Fire Fly Luciferase is measured using VLUX (60 second exposure) immediately after the substrate was added. Test compounds are dissolved in DMSO (100%) and added to specified wells. Raw luciferase signal data obtained as relative luminescence units (RLUs) for the test compounds are normalized to the signal of the mean of the sample reporter cell plate with DMSO.

Active compounds have TCF-luciferase ratios of 2.5 fold or greater over DMSO. All compounds show a signal increase of at least 10% compared to a signal with only DMSO added. Results from the above biologic procedures of example compounds are shown in the following table:

### A=MAX FOLD Induction/DMSO CONTROL

B=TCF ACTIVITY/DMSO CONTROL Fold induction @ 2.0 uM
C=TCF ACTIVITY/DMSO CONTROL Fold induction @ 20.0 uM

| | EC 50 | | | |
|---|---|---|---|---|
| Compound | (μm) | A | B | C |
| 7-(2-naphthyl)-3-piperidin-4-yl-2-pyridin-2-yl-3H-imidazo[4,5-b]pyridine | | | 1.120 | 0.020 |
| 7-(2-naphthyl)-3-piperidin-4-yl-2-pyridin-3-yl-3H-imidazo[4,5-b]pyridine | | | 1.210 | 0.000 |
| 7-(2-naphthyl)-3-piperidin-4-yl-2-pyridin-4-yl-3H-imidazo[4,5-b]pyridine | | 1.650 | 1.890 | 2.270 |
| 7-(2-naphthyl)-3-piperidin-4-yl-2-(1H-pyrrol-2-yl)-3H-imidazo[4,5-b]pyridine | | | 1.250 | 0.000 |
| 2-(1-methyl-1H-indol-2-yl)-7-(2-naphthyl)-3-piperidin-4-yl-3H-imidazo[4,5- | | | 1.170 | 0.000 |
| b]pyridine | | | | |
| 7-(2-naphthyl)-3-piperidin-4-yl-2-(2-thienyl)-3H-imidazo[4,5-b]pyridine | | | 1.220 | 0.040 |
| 2-(4-morpholin-4-yl-3-nitrophenyl)-7-(2-naphthyl)-3-piperidin-4-yl-3H-imidazo[4,5- | | | 1.440 | 0.060 |
| b]pyridine | | | | |
| 8-[7-(2-naphthyl)-3-piperidin-4-yl-3H-imidazo[4,5-b]pyridin-2-yl]quinoline | | | 0.000 | 1.010 |
| 5-[7-(2-naphthyl)-3-piperidin-4-yl-3H-imidazo[4,5-b]pyridin-2-yl]quinoline | | | 1.330 | 0.000 |
| 2-(1-benzofuran-5-yl)-7-(2-naphthyl)-3-piperidin-4-yl-3H-imidazo[4,5-b]pyridine | | | 0.900 | 1.020 |
| 2-(5-methyl-2-furyl)-7-(2-naphthyl)-3-piperidin-4-yl-3H-imidazo[4,5-b]pyridine | | | 1.170 | 0.000 |
| 2-(1-methyl-1H-indol-3-yl)-7-(2-naphthyl)-3-piperidin-4-yl-3H-imidazo[4,5- | | | 1.230 | 0.000 |
| b]pyridine | | | | |
| 2-(2-methoxypyridin-3-yl)-7-(2-naphthyl)-3-piperidin-4-yl-3H-imidazo[4,5-b]pyridine | | | 1.500 | 0.000 |
| 4-[7-(2-naphthyl)-3-piperidin-4-yl-3H-imidazo[4,5-b]pyridin-2-yl]quinoline | | | 1.070 | 0.000 |
| 2-(2-furyl)-7-(2-naphthyl)-3-piperidin-4-yl-3H-imidazo[4,5-b]pyridine | | | 1.820 | 0.010 |
| 2-(1-benzofuran-2-yl)-7-(2-naphthyl)-3-piperidin-4-yl-3H-imidazo[4,5-b]pyridine | | | 1.530 | 0.010 |
| 2-(1-benzothien-2-yl)-7-(2-naphthyl)-3-piperidin-4-yl-3H-imidazo[4,5-b]pyridine | | | 1.020 | 0.000 |
| 2-isobutyl-7-(2-naphthyl)-3-piperidin-4-yl-3H-imidazo[4,5-b]pyridine | | | 1.050 | 0.000 |
| 2-cyclohexyl-7-(2-naphthyl)-3-piperidin-4-yl-3H-imidazo[4,5-b]pyridine | | | 1.880 | 0.000 |
| 7-(2-naphthyl)-3-piperidin-4-yl-2-propyl-3H-imidazo[4,5-b]pyridine | | | 1.930 | 0.000 |
| 2-butyl-7-(2-naphthyl)-3-piperidin-4-yl-3H-imidazo[4,5-b]pyridine | | | 1.190 | 0.000 |
| 2-ethyl-7-(2-naphthyl)-3-piperidin-4-yl-3H-imidazo[4,5-b]pyridine | | | 1.390 | 0.000 |
| 2-(4-isopropylphenyl)-7-(2-naphthyl)-3-piperidin-4-yl-3H-imidazo[4,5-b]pyridine | | | 1.030 | 0.000 |
| 2-(4-tert-butylphenyl)-7-(2-naphthyl)-3-piperidin-4-yl-3H-imidazo[4,5-b]pyridine | | | 1.880 | 0.000 |
| 2-(3-methoxyphenyl)-7-(2-naphthyl)-3-piperidin-4-yl-3H-imidazo[4,5-b]pyridine | | | 1.250 | 0.000 |
| 7-(2-naphthyl)-2-(2-nitrophenyl)-3-piperidin-4-yl-3H-imidazo[4,5-b]pyridine | | | 1.140 | 0.000 |
| 2-(4-methylphenyl)-7-(2-naphthyl)-3-piperidin-4-yl-3H-imidazo[4,5-b]pyridine | | | 1.900 | 0.000 |
| 7-(2-naphthyl)-2-(3-nitrophenyl)-3-piperidin-4-yl-3H-imidazo[4,5-b]pyridine | | | 1.295 | 0.000 |
| 7-(2-naphthyl)-2-phenyl-3-piperidin-4-yl-3H-imidazo[4,5-b]pyridine | | | 1.230 | 0.050 |
| 2-(2-methoxyphenyl)-7-(2-naphthyl)-3-piperidin-4-yl-3H-imidazo[4,5-b]pyridine | | | 1.990 | 0.000 |
| 2-(3-methylphenyl)-7-(2-naphthyl)-3-piperidin-4-yl-3H-imidazo[4,5-b]pyridine | | | 1.910 | 0.000 |
| 2-(2,4-dimethylphenyl)-7-(2-naphthyl)-3-piperidin-4-yl-3H-imidazo[4,5-b]pyridine | | | 1.300 | 0.000 |
| 2-(2,4-dichlorophenyl)-7-(2-naphthyl)-3-piperidin-4-yl-3H-imidazo[4,5-b]pyridine | | | 1.100 | 0.000 |
| 2-(1-naphthyl)-7-(2-naphthyl)-3-piperidin-4-yl-3H-imidazo[4,5-b]pyridine | | | 1.390 | 0.000 |
| 2,7-di-2-naphthyl-3-piperidin-4-yl-3H-imidazo[4,5-b]pyridine | | | 1.020 | 0.000 |
| 2-(2,4-difluorophenyl)-7-(2-naphthyl)-3-piperidin-4-yl-3H-imidazo[4,5-b]pyridine | | | 1.420 | 0.000 |
| 2-(3-chlorophenyl)-7-(2-naphthyl)-3-piperidin-4-yl-3H-imidazo[4,5-b]pyridine | | | 1.500 | 0.000 |
| 2-(2-fluorophenyl)-7-(2-naphthyl)-3-piperidin-4-yl-3H-imidazo[4,5-b]pyridine | | | 1.290 | 0.000 |
| 4-[7-(2-naphthyl)-3-piperidin-4-yl-3H-imidazo[4,5-b]pyridin-2-yl]phenol | | | 1.300 | 0.000 |
| 3-[7-(2-naphthyl)-3-piperidin-4-yl-3H-imidazo[4,5-b]pyridin-2-yl]phenol | | | 1.580 | 0.000 |
| 2-[7-(2-naphthyl)-3-piperidin-4-yl-3H-imidazo[4,5-b]pyridin-2-yl]phenol | | | 1.350 | 0.010 |
| 3-[7-(2-naphthyl)-3-piperidin-4-yl-3H-imidazo[4,5-b]pyridin-2-yl]benzene-1,2-diol | | | 1.330 | 0.000 |
| 2-methoxy-4-[7-(2-naphthyl)-3-piperidin-4-yl-3H-imidazo[4,5-b]pyridin-2-yl]phenol | | | 1.630 | 0.010 |
| 3-[7-(2-naphthyl)-3-piperidin-4-yl-3H-imidazo[4,5-b]pyridin-2-yl]-4-nitrophenol | | | 1.400 | 0.980 |
| 2-(4-methoxyphenyl)-7-(2-naphthyl)-3-piperidin-4-yl-3H-imidazo[4,5-b]pyridine | | 1.320 | 1.980 | 0.000 |
| 2-(2-bromophenyl)-7-(2-naphthyl)-3-piperidin-4-yl-3H-imidazo[4,5-b]pyridine | | | 1.600 | 0.000 |
| 2-(3-bromophenyl)-7-(2-naphthyl)-3-piperidin-4-yl-3H-imidazo[4,5-b]pyridine | | | 1.520 | 0.000 |
| 2-(4-bromophenyl)-7-(2-naphthyl)-3-piperidin-4-yl-3H-imidazo[4,5-b]pyridine | | | 1.230 | 0.000 |
| 7-(2-naphthyl)-3-piperidin-4-yl-2-(3-vinylphenyl)-3H-imidazo[4,5-b]pyridine | | | 1.440 | 0.000 |
| 4-[7-(2-naphthyl)-3-piperidin-4-yl-3H-imidazo[4,5-b]pyridin-2-yl]-N,N- | | | 1.890 | 0.000 |
| diphenylaniline | | | | |
| 2-(2-methoxy-1-naphthyl)-7-(2-naphthyl)-3-piperidin-4-yl-3H-imidazo[4,5-b]pyridine | | | 1.170 | 0.000 |
| 7-(2-naphthyl)-2-(3-phenoxyphenyl)-3-piperidin-4-yl-3H-imidazo[4,5-b]pyridine | | | 1.400 | 0.000 |
| 7-(2-naphthyl)-3-piperidin-4-yl-2-{3-[3-(trifluoromethyl)phenoxy]phenyl}-3H- | | | 1.290 | 0.000 |
| imidazo[4,5-b]pyridine | | | | |
| 7-(2-naphthyl)-3-piperidin-4-yl-2-[4-(trifluoromethyl)phenyl]-3H-imidazo[4,5- | | | 1.280 | 0.000 |
| b]pyridine | | | | |
| 7-(2-naphthyl)-3-piperidin-4-yl-2-[3-(trifluoromethyl)phenyl]-3H-imidazo[4,5- | | | 1.540 | 0.000 |
| b]pyridine | | | | |
| 7-(2-naphthyl)-3-piperidin-4-yl-2-[2-(trifluoromethyl)phenyl]-3H-imidazo[4,5- | | | 1.720 | 0.000 |
| b]pyridine | | | | |
| 2-(2,6-difluorophenyl)-7-(2-naphthyl)-3-piperidin-4-yl-3H-imidazo[4,5-b]pyridine | | | 1.110 | 0.000 |
| 2-(2,3-difluorophenyl)-7-(2-naphthyl)-3-piperidin-4-yl-3H-imidazo[4,5-b]pyridine | | | 1.900 | 0.000 |
| 7-(2-naphthyl)-3-piperidin-4-yl-2-[4-(trifluoromethoxy)phenyl]-3H-imidazo[4,5- | | | 1.510 | 0.000 |
| b]pyridine | | | | |
| 7-(2-naphthyl)-3-piperidin-4-yl-2-[2-(trifluoromethoxy)phenyl]-3H-imidazo[4,5- | | | 1.880 | 0.000 |
| b]pyridine | | | | |
| 2-[2,5-bis(trifluoromethyl)phenyl]-7-(2-naphthyl)-3-piperidin-4-yl-3H-imidazo[4,5- | | | 1.450 | 0.000 |
| b]pyridine | | | | |
| 2-[4-fluoro-2-(trifluoromethyl)phenyl]-7-(2-naphthyl)-3-piperidin-4-yl-3H- | | | 1.110 | 0.000 |
| imidazo[4,5-b]pyridine | | | | |
| 2-[2-chloro-3-(trifluoromethyl)phenyl]-7-(2-naphthyl)-3-piperidin-4-yl-3H- | | | 1.210 | 0.000 |
| imidazo[4,5-b]pyridine | | | | |
| 2-(3-fluorophenyl)-7-(2-naphthyl)-3-piperidin-4-yl-3H-imidazo[4,5-b]pyridine | | | 1.550 | 0.000 |
| 2-(2-chlorophenyl)-7-(2-naphthyl)-3-piperidin-4-yl-3H-imidazo[4,5-b]pyridine | | | 1.360 | 0.000 |
| 2-(4-fluorophenyl)-7-(2-naphthyl)-3-piperidin-4-yl-3H-imidazo[4,5-b]pyridine | | 1.370 | 2.070 | 0.000 |
| 4-[7-(2-naphthyl)-3-piperidin-4-yl-3H-imidazo[4,5-b]pyridin-2-yl]benzonitrile | | | 1.580 | 0.000 |
| N-{4-[7-(2-naphthyl)-3-piperidin-4-yl-3H-imidazo[4,5-b]pyridin-2- | | 1.710 | 1.990 | 0.000 |
| yl]phenyl}acetamide | | | | |
| 7-(2-naphthyl)-2-(4-nitrophenyl)-3-piperidin-4-yl-3H-imidazo[4,5-b]pyridine | | | 1.420 | 0.000 |
| 2-(4-chlorophenyl)-7-(2-naphthyl)-3-piperidin-4-yl-3H-imidazo[4,5-b]pyridine | | | 1.210 | 0.000 |
| 2-(1-ethyl-5-methyl-1H-imidazol-4-yl)-7-(2-naphthyl)-3-piperidin-4-yl-3H- | | | 1.880 | 0.000 |
| imidazo[4,5-b]pyridine | | | | |
| 2-(1-methyl-1H-imidazol-2-yl)-7-(2-naphthyl)-3-piperidin-4-yl-3H-imidazo[4,5- | | | 1.840 | 0.000 |
| b]pyridine | | | | |
| 2-(4-methyl-1H-imidazol-5-yl)-7-(2-naphthyl)-3-piperidin-4-yl-3H-imidazo[4,5- | | | 1.310 | 0.000 |
| b]pyridine | | | | |
| 2-(1H-imidazol-2-yl)-7-(2-naphthyl)-3-piperidin-4-yl-3H-imidazo[4,5-b]pyridine | | | 1.420 | 0.000 |
| 2-(2-methyl-1H-imidazol-4-yl)-7-(2-naphthyl)-3-piperidin-4-yl-3H-imidazo[4,5- | | | 1.040 | 0.020 |
| b]pyridine | | | | |
| 7-(2-naphthyl)-2-(5-nitro-2-thienyl)-3-piperidin-4-yl-3H-imidazo[4,5-b]pyridine | | | 1.830 | 0.010 |
| 7-(2-naphthyl)-3-piperidin-4-yl-2-(3-thienyl)-3H-imidazo[4,5-b]pyridine | | | 1.170 | 0.010 |
| 2-(1-benzothien-3-yl)-7-(2-naphthyl)-3-piperidin-4-yl-3H-imidazo[4,5-b]pyridine | | | 1.280 | 0.000 |
| 2-(5-methoxy-1H-indol-3-yl)-7-(2-naphthyl)-3-piperidin-4-yl-3H-imidazo[4,5- | | | 1.200 | 0.000 |
| b]pyridine | | | | |
| 2-(5-methyl-1H-indol-3-yl)-7-(2-naphthyl)-3-piperidin-4-yl-3H-imidazo[4,5- | | | 1.920 | 0.000 |
| b]pyridine | | | | |
| 2-(1-acetyl-1H-indol-3-yl)-7-(2-naphthyl)-3-piperidin-4-yl-3H-imidazo[4,5-b]pyridine | | | 1.480 | 0.000 |
| 2-(4,5-dimethyl-2-furyl)-7-(2-naphthyl)-3-piperidin-4-yl-3H-imidazo[4,5-b]pyridine | | | 1.540 | 0.000 |
| 7-(2-naphthyl)-2-(1-oxidopyridin-4-yl)-3-piperidin-4-yl-3H-imidazo[4,5-b]pyridine | | | 1.450 | 0.060 |
| 2-(3,5-dichloropyridin-4-yl)-7-(2-naphthyl)-3-piperidin-4-yl-3H-imidazo[4,5- | | | 1.950 | 0.290 |
| b]pyridine | | | | |
| 2-(1-methyl-1H-pyrrol-2-yl)-7-(2-naphthyl)-3-piperidin-4-yl-3H-imidazo[4,5- | | | 1.520 | 0.010 |
| b]pyridine | | | | |
| 5-[7-(2-naphthyl)-3-piperidin-4-yl-3H-imidazo[4,5-b]pyridin-2-yl]pyrimidine- | | | 1.940 | 1.910 |
| 2,4(1H,3H)-dione | | | | |
| 2-(1,3-benzodioxol-5-yl)-7-(2-naphthyl)-3-piperidin-4-yl-3H-imidazo[4,5-b]pyridine | | | 1.360 | 0.000 |
| 2-(2,3-dihydro-1,4-benzodioxin-6-yl)-7-(2-naphthyl)-3-piperidin-4-yl-3H- | | | 1.180 | 0.000 |
| imidazo[4,5-b]pyridine | | | | |
| 2-(2-chloro-4-methyl-1-phenyl-1H-pyrrol-3-yl)-7-(2-naphthyl)-3-piperidin-4-yl-3H- | | | 1.480 | 0.000 |
| imidazo[4,5-b]pyridine | | | | |
| 2-chloro-3-[7-(2-naphthyl)-3-piperidin-4-yl-3H-imidazo[4,5-b]pyridin-2-yl]quinoline | | | 1.600 | 0.000 |
| 3-[7-(2-naphthyl)-3-piperidin-4-yl-3H-imidazo[4,5-b]pyridin-2-yl]quinoline | | | 1.440 | 0.000 |
| 2-[7-(2-naphthyl)-3-piperidin-4-yl-3H-imidazo[4,5-b]pyridin-2-yl]quinoline | | | 0.010 | 1.010 |
| 2-cyclopentyl-7-(2-naphthyl)-3-piperidin-4-yl-3H-imidazo[4,5-b]pyridine | | | 1.460 | 0.000 |
| 7-(2-naphthyl)-3-piperidin-4-yl-2-[3-(trifluoromethoxy)phenyl]-3H-imidazo[4,5- | | | 1.520 | 0.000 |
| b]pyridine | | | | |
| 7-(2-naphthyl)-3-piperidin-4-yl-3H-imidazo[4,5-b]pyridine | 0.725 | 3.881 | 5.403 | 0.063 |
| 2-methyl-7-(2-naphthyl)-3-piperidin-4-yl-3H-imidazo[4,5-b]pyridine | 0.895 | 3.215 | 2.840 | 0.000 |
| 7-(2-naphthyl)-3-[1-(trifluoroacetyl)piperidin-4-yl]-3H-imidazo[4,5-b]pyridine | | | 1.580 | 0.390 |
| methyl 4-[7-(2-naphthyl)-3H-imidazo[4,5-b]pyridin-3-yl]piperidine-1-carboxylate | | | 1.080 | 1.020 |
| N,N-dimethyl-4-[7-(2-naphthyl)-3H-imidazo[4,5-b]pyridin-3-yl]piperidine-1- | | 1.080 | 1.940 | 2.090 |
| carboxamide | | | | |
| N-ethyl-4-[7-(2-naphthyl)-3H-imidazo[4,5-b]pyridin-3-yl]piperidine-1-carboxamide | | | 1.520 | 1.290 |
| N-isopropyl-4-[7-(2-naphthyl)-3H-imidazo[4,5-b]pyridin-3-yl]piperidine-1- | | | 1.200 | 0.840 |
| carboxamide | | | | |
| 3-(1-benzylpiperidin-4-yl)-7-(2-naphthyl)-3H-imidazo[4,5-b]pyridine | | | 0.840 | 0.080 |
| 3-[1-(methylsulfonyl)piperidin-4-yl]-7-(2-naphthyl)-3H-imidazo[4,5-b]pyridine | | 1.230 | 1.660 | 2.520 |
| N-cyclohexyl-4-[7-(2-naphthyl)-3H-imidazo[4,5-b]pyridin-3-yl]piperidine-1- | | | 0.970 | 0.030 |
| carboxamide | | | | |
| 3-[1-(morpholin-4-ylcarbonyl)piperidin-4-yl]-7-(2-naphthyl)-3H-imidazo[4,5- | | | 1.080 | 1.250 |
| b]pyridine | | | | |
| N-(3′,6′-dihydroxy-3-oxo-3H-spiro[2-benzofuran-1,9′-xanthen]-5-yl)-4-[7-(2- | 10.720 | 12.158 | 1.400 | 6.490 |
| naphthyl)-3H-imidazo[4,5-b]pyridin-3-yl]piperidine-1-carbothioamide | | | | |
| N,N-dimethyl-4-[7-(2-naphthyl)-3H-imidazo[4,5-b]pyridin-3-yl]piperidine-1- | | | 1.190 | 1.460 |
| sulfonamide | | | | |
| 7-(2-naphthyl)-3-(piperidin-4-ylmethyl)-3H-imidazo[4,5-b]pyridine | | 1.930 | 2.720 | 0.000 |
| 3-[3-(1H-imidazol-1-yl)propyl]-7-(2-naphthyl)-3H-imidazo[4,5-b]pyridine | 10.190 | 2.870 | 1.760 | 3.310 |
| 2-methyl-7-(2-naphthyl)-3-[(3R)-pyrrolidin-3-yl]-3H-imidazo[4,5-b]pyridine | | | 1.440 | 0.550 |
| 2-methyl-3-[2-(4-methylphenyl)ethyl]-7-(2-naphthyl)-3H-imidazo[4,5-b]pyridine | | | 0.820 | 1.000 |
| 7-(2-naphthyl)-3-[(3S)-piperidin-3-yl]-3H-imidazo[4,5-b]pyridine | 0.018 | 2.303 | 1.020 | 0.010 |
| 3-[2-(4-methylphenyl)ethyl]-7-(2-naphthyl)-3H-imidazo[4,5-b]pyridine | | | 0.760 | 0.980 |
| 2-methyl-7-(2-naphthyl)-3-(piperidin-4-ylmethyl)-3H-imidazo[4,5-b]pyridine | | | 1.120 | 0.080 |
| 3-azetidin-3-yl-7-(2-naphthyl)-3H-imidazo[4,5-b]pyridine | | | 1.490 | 1.250 |
| 7-(2-naphthyl)-3-[(3R)-pyrrolidin-3-yl]-3H-imidazo[4,5-b]pyridine | 3.150 | 4.260 | 2.060 | 1.650 |
| 7-(2-naphthyl)-3-[(3S)-pyrrolidin-3-yl]-3H-imidazo[4,5-b]pyridine | | 2.780 | 2.100 | 0.440 |
| N-isopropyl-4-[7-(2-naphthyl)-2-oxo-1,2-dihydro-3H-imidazo[4,5-b]pyridin-3- | | | 1.420 | 1.100 |
| yl]piperidine-1-carboxamide | | | | |
| 3-(1-acetylpiperidin-4-yl)-1-methyl-7-(2-naphthyl)-1,3-dihydro-2H-imidazo[4,5- | | | 1.090 | 0.510 |
| b]pyridin-2-one | | | | |
| N,N-dimethyl-4-[1-methyl-7-(2-naphthyl)-2-oxo-1,2-dihydro-3H-imidazo[4,5- | | | 1.190 | 0.350 |
| b]pyridin-3-yl]piperidine-1-carboxamide | | | | |
| 2-methyl-7-(2-naphthyl)-3-[(3S)-pyrrolidin-3-yl]-3H-imidazo[4,5-b]pyridine | | | 1.570 | 0.400 |
| 2-methyl-7-(2-naphthyl)-3-[(3R)-piperidin-3-yl]-3H-imidazo[4,5-b]pyridine | 2.680 | 3.570 | 2.740 | 0.050 |
| 3-[1-(morpholin-4-ylcarbonyl)piperidin-4-yl]-7-(2-naphthyl)-1,3-dihydro-2H- | | | 1.330 | 1.020 |
| imidazo[4,5-b]pyridin-2-one | | | | |
| 7-(2-naphthyl)-3-[(3R)-piperidin-3-yl]-3H-imidazo[4,5-b]pyridine | 0.963 | 3.820 | 3.485 | 1.395 |
| 3-azetidin-3-yl-2-methyl-7-(2-naphthyl)-3H-imidazo[4,5-b]pyridine | | | 1.160 | 0.760 |
| 2-methyl-7-(2-naphthyl)-3-[(3S)-piperidin-3-yl]-3H-imidazo[4,5-b]pyridine | | 1.910 | 2.120 | 0.020 |
| N-isopropyl-4-[1-methyl-7-(2-naphthyl)-2-oxo-1,2-dihydro-3H-imidazo[4,5- | | | 1.070 | 0.260 |
| b]pyridin-3-yl]piperidine-1-carboxamide | | | | |
| 6-(2-naphthyl)-3-piperidin-4-yl-2-(1H-pyrrol-2-yl)-3H-imidazo[4,5-b]pyridine | | 1.580 | 1.010 | 1.990 |
| 2-(1-methyl-1H-indol-2-yl)-6-(2-naphthyl)-3-piperidin-4-yl-3H-imidazo[4,5- | | | 1.055 | 0.000 |
| b]pyridine | | | | |
| 2-(1H-indol-3-yl)-6-(2-naphthyl)-3-piperidin-4-yl-3H-imidazo[4,5-b]pyridine | | | 1.110 | 0.000 |
| 6-(2-naphthyl)-3-piperidin-4-yl-3H-imidazo[4,5-b]pyridine | | | 1.530 | 0.000 |
| 2-methyl-6-(2-naphthyl)-3-piperidin-4-yl-3H-imidazo[4,5-b]pyridine | | | 1.060 | 1.170 |
| 4-[6-(2-naphthyl)-3-piperidin-4-yl-3H-imidazo[4,5-b]pyridin-2-yl]quinoline | | | 1.810 | 0.000 |
| 2-(4-isopropylphenyl)-6-(2-naphthyl)-3-piperidin-4-yl-3H-imidazo[4,5-b]pyridine | | | 1.140 | 0.000 |
| 2-(2,4-dichlorophenyl)-6-(2-naphthyl)-3-piperidin-4-yl-3H-imidazo[4,5-b]pyridine | | | 1.040 | 0.000 |
| 2-(3-chlorophenyl)-6-(2-naphthyl)-3-piperidin-4-yl-3H-imidazo[4,5-b]pyridine | | | 0.980 | 0.000 |
| 6-(2-naphthyl)-3-piperidin-4-yl-2-pyridin-4-yl-3H-imidazo[4,5-b]pyridine | | | 1.560 | 0.005 |
| 6-(2-naphthyl)-3-piperidin-4-yl-2-(2-thienyl)-3H-imidazo[4,5-b]pyridine | | | 1.040 | 0.000 |
| 2-(2-methoxypyridin-3-yl)-6-(2-naphthyl)-3-piperidin-4-yl-3H-imidazo[4,5-b]pyridine | | 2.030 | 2.060 | 0.020 |
| 6-(2-naphthyl)-2-(3-nitrophenyl)-3-piperidin-4-yl-3H-imidazo[4,5-b]pyridine | | | 1.300 | 0.000 |
| 2-ethyl-6-(2-naphthyl)-3-piperidin-4-yl-3H-imidazo[4,5-b]pyridine | | | 1.270 | 0.780 |
| 1-methyl-3-[1-(morpholin-4-ylcarbonyl)piperidin-4-yl]-7-(2-naphthyl)-1,3-dihydro- | | | 1.290 | 0.210 |
| 2H-imidazo[4,5-b]pyridin-2-one | | | | |
| 1-methyl-3-[1-(methylsulfonyl)piperidin-4-yl]-7-(2-naphthyl)-1,3-dihydro-2H- | | | 1.520 | 0.970 |
| imidazo[4,5-b]pyridin-2-one | | | | |
| 6-(2-naphthyl)-3-piperidin-4-yl-2-pyridin-2-yl-3H-imidazo[4,5-b]pyridine | | | 1.605 | 0.005 |
| 2-(3-methoxyphenyl)-6-(2-naphthyl)-3-piperidin-4-yl-3H-imidazo[4,5-b]pyridine | | | 0.970 | 0.000 |
| 2-(4-methylphenyl)-6-(2-naphthyl)-3-piperidin-4-yl-3H-imidazo[4,5-b]pyridine | | | 1.000 | 0.000 |
| 4-[6-(2-naphthyl)-3-piperidin-4-yl-3H-imidazo[4,5-b]pyridin-2-yl]phenol | | 1.710 | | |
| 2-butyl-6-(2-naphthyl)-3-piperidin-4-yl-3H-imidazo[4,5-b]pyridine | | | 1.210 | 0.000 |
| 2-(2-fluorophenyl)-6-(2-naphthyl)-3-piperidin-4-yl-3H-imidazo[4,5-b]pyridine | | | 1.290 | 0.000 |
| 2-isopropyl-6-(2-naphthyl)-3-piperidin-4-yl-3H-imidazo[4,5-b]pyridine | | | 1.440 | 0.000 |
| 2-(2-furyl)-6-(2-naphthyl)-3-piperidin-4-yl-3H-imidazo[4,5-b]pyridine | | | 1.270 | 0.010 |
| 2-(1-benzothien-2-yl)-6-(2-naphthyl)-3-piperidin-4-yl-3H-imidazo[4,5-b]pyridine | | | 0.920 | 0.000 |
| 2-cyclohexyl-6-(2-naphthyl)-3-piperidin-4-yl-3H-imidazo[4,5-b]pyridine | | | 1.240 | 0.010 |
| 6-(2-naphthyl)-3-piperidin-4-yl-2-propyl-3H-imidazo[4,5-b]pyridine | | | 1.380 | 0.010 |
| 2-(2-methoxyphenyl)-6-(2-naphthyl)-3-piperidin-4-yl-3H-imidazo[4,5-b]pyridine | | | 1.370 | 0.010 |
| 2-(2,4-difluorophenyl)-6-(2-naphthyl)-3-piperidin-4-yl-3H-imidazo[4,5-b]pyridine | | | 1.220 | 0.000 |
| 2-(1-benzofuran-5-yl)-6-(2-naphthyl)-3-piperidin-4-yl-3H-imidazo[4,5-b]pyridine | | | 0.980 | 0.000 |
| 2-(5-methyl-2-furyl)-6-(2-naphthyl)-3-piperidin-4-yl-3H-imidazo[4,5-b]pyridine | | | 1.230 | 0.000 |
| 2-(1-benzofuran-2-yl)-6-(2-naphthyl)-3-piperidin-4-yl-3H-imidazo[4,5-b]pyridine | | | 1.000 | 0.000 |
| 2-(4-tert-butylphenyl)-6-(2-naphthyl)-3-piperidin-4-yl-3H-imidazo[4,5-b]pyridine | | | 1.130 | 0.000 |
| 2-(2,4-dimethylphenyl)-6-(2-naphthyl)-3-piperidin-4-yl-3H-imidazo[4,5-b]pyridine | | | 1.310 | 0.000 |
| 2-(1-naphthyl)-6-(2-naphthyl)-3-piperidin-4-yl-3H-imidazo[4,5-b]pyridine | | | 1.110 | 0.000 |
| 3-[6-(2-naphthyl)-3-piperidin-4-yl-3H-imidazo[4,5-b]pyridin-2-yl]phenol | 1.220 | 1.860 | | |
| 6-(2-naphthyl)-3-piperidin-4-yl-2-pyridin-3-yl-3H-imidazo[4,5-b]pyridine | | 1.100 | 1.755 | 0.005 |
| 2-(4-morpholin-4-yl-3-nitrophenyl)-6-(2-naphthyl)-3-piperidin-4-yl-3H-imidazo[4,5- | | | 1.010 | 0.000 |
| b]pyridine | | | | |
| 8-[6-(2-naphthyl)-3-piperidin-4-yl-3H-imidazo[4,5-b]pyridin-2-yl]quinoline | | | 1.610 | 0.000 |
| 5-[6-(2-naphthyl)-3-piperidin-4-yl-3H-imidazo[4,5-b]pyridin-2-yl]quinoline | | | 1.290 | 0.000 |
| 2-(1-methyl-1H-indol-3-yl)-6-(2-naphthyl)-3-piperidin-4-yl-3H-imidazo[4,5- | | | 1.040 | 0.000 |
| b]pyridine | | | | |
| 2-isobutyl-6-(2-naphthyl)-3-piperidin-4-yl-3H-imidazo[4,5-b]pyridine | | | 1.130 | 0.000 |
| 6-(2-naphthyl)-2-(2-nitrophenyl)-3-piperidin-4-yl-3H-imidazo[4,5-b]pyridine | | | 1.130 | 0.000 |
| 6-(2-naphthyl)-2-phenyl-3-piperidin-4-yl-3H-imidazo[4,5-b]pyridine | | | 1.600 | 0.000 |
| 2-(3-methylphenyl)-6-(2-naphthyl)-3-piperidin-4-yl-3H-imidazo[4,5-b]pyridine | | | 1.330 | 0.000 |
| 2,6-di-2-naphthyl-3-piperidin-4-yl-3H-imidazo[4,5-b]pyridine | | | 1.060 | 0.000 |
| tert-butyl (3R)-3-[7-(2-naphthyl)-3H-imidazo[4,5-b]pyridin-3-yl]pyrrolidine-1- | | | | |
| carboxylate | | | | |
| tert-butyl (3S)-3-[7-(2-naphthyl)-3H-imidazo[4,5-b]pyridin-3-yl]pyrrolidine-1- | | | | |
| carboxylate | | | | |
| 6-(2-naphthyl)-3-piperidin-4-yl-2-[2-(trifluoromethyl)phenyl]-3H-imidazo[4,5- | | 1.360 | | |
| b]pyridine | | | | |
| 6-(2-naphthyl)-3-piperidin-4-yl-2-[4-(trifluoromethoxy)phenyl]-3H-imidazo[4,5- | | | | |
| b]pyridine | | | | |
| 4-[6-(2-naphthyl)-3-piperidin-4-yl-3H-imidazo[4,5-b]pyridin-2-yl]benzonitrile | | | | |
| 2-(1H-imidazol-2-yl)-6-(2-naphthyl)-3-piperidin-4-yl-3H-imidazo[4,5-b]pyridine | | | | |
| 2-(3,5-dichloropyridin-4-yl)-6-(2-naphthyl)-3-piperidin-4-yl-3H-imidazo[4,5- | | | | |
| b]pyridine | | | | |
| 2-(1,3-benzodioxol-5-yl)-6-(2-naphthyl)-3-piperidin-4-yl-3H-imidazo[4,5-b]pyridine | 0.737 | 1.860 | | |
| tert-butyl (3S)-3-[2-methyl-7-(2-naphthyl)-3H-imidazo[4,5-b]pyridin-3-yl]piperidine- | | | | |
| 1-carboxylate | | | | |
| 5-(2-naphthyl)-3-piperidin-4-yl-3H-imidazo[4,5-b]pyridine | | | 0.320 | 0.000 |
| 4-[6-(2-naphthyl)-3-piperidin-4-yl-3H-imidazo[4,5-b]pyridin-2-yl]-N,N- | | | | |
| diphenylaniline | | | | |
| 6-(2-naphthyl)-3-piperidin-4-yl-2-[4-(trifluoromethyl)phenyl]-3H-imidazo[4,5- | | | | |
| b]pyridine | | | | |
| 2-[2-chloro-3-(trifluoromethyl)phenyl]-6-(2-naphthyl)-3-piperidin-4-yl-3H- | | | | |
| imidazo[4,5-b]pyridine | | | | |
| 6-(2-naphthyl)-2-(4-nitrophenyl)-3-piperidin-4-yl-3H-imidazo[4,5-b]pyridine | | | | |
| 2-(2-methyl-1H-imidazol-4-yl)-6-(2-naphthyl)-3-piperidin-4-yl-3H-imidazo[4,5- | | | | |
| b]pyridine | | | | |
| 6-(2-naphthyl)-3-piperidin-4-yl-2-[3-(trifluoromethoxy)phenyl]-3H-imidazo[4,5- | | | | |
| b]pyridine | | | | |
| tert-butyl 3-[7-(2-naphthyl)-3H-imidazo[4,5-b]pyridin-3-yl]azetidine-1-carboxylate | | | | |
| tert-butyl (3R)-3-[7-(2-naphthyl)-3H-imidazo[4,5-b]pyridin-3-yl]piperidine-1- | | | | |
| carboxylate | | | | |
| tert-butyl (3R)-3-[2-methyl-7-(2-naphthyl)-3H-imidazo[4,5-b]pyridin-3-yl]piperidine- | | | | |
| 1-carboxylate | | | | |
| 2-(4-methoxyphenyl)-6-(2-naphthyl)-3-piperidin-4-yl-3H-imidazo[4,5-b]pyridine | | 1.350 | | |
| 6-(2-naphthyl)-3-piperidin-4-yl-2-[2-(trifluoromethoxy)phenyl]-3H-imidazo[4,5- | | | | |
| b]pyridine | | | | |
| 2-(2-chlorophenyl)-6-(2-naphthyl)-3-piperidin-4-yl-3H-imidazo[4,5-b]pyridine | | | | |
| 2-(4-methyl-1H-imidazol-5-yl)-6-(2-naphthyl)-3-piperidin-4-yl-3H-imidazo[4,5- | | 1.870 | | |
| b]pyridine | | | | |
| 2-(1-acetyl-1H-indol-3-yl)-6-(2-naphthyl)-3-piperidin-4-yl-3H-imidazo[4,5-b]pyridine | | | | |
| 2-(2,3-dihydro-1,4-benzodioxin-6-yl)-6-(2-naphthyl)-3-piperidin-4-yl-3H- | | 1.460 | | |
| imidazo[4,5-b]pyridine | | | | |
| 2-chloro-3-[6-(2-naphthyl)-3-piperidin-4-yl-3H-imidazo[4,5-b]pyridin-2-yl]quinoline | | | | |
| 7-(2-naphthyl)-3-piperidin-4-yl-3H-imidazo[4,5-b]pyridine | 0.725 | 3.881 | 5.403 | 0.063 |
| 2-methyl-7-(2-naphthyl)-3-piperidin-4-yl-3H-imidazo[4,5-b]pyridine | 0.895 | 3.215 | 2.840 | 0.000 |
| 3-[6-(2-naphthyl)-3-piperidin-4-yl-3H-imidazo[4,5-b]pyridin-2-yl]benzene-1,2-diol | | 1.430 | | |
| 3-[6-(2-naphthyl)-3-piperidin-4-yl-3H-imidazo[4,5-b]pyridin-2-yl]-4-nitrophenol | | | | |
| 2-(2,6-difluorophenyl)-6-(2-naphthyl)-3-piperidin-4-yl-3H-imidazo[4,5-b]pyridine | | | | |
| 2-[4-fluoro-2-(trifluoromethyl)phenyl]-6-(2-naphthyl)-3-piperidin-4-yl-3H- | | | | |
| imidazo[4,5-b]pyridine | | | | |
| 2-(1-ethyl-5-methyl-1H-imidazol-4-yl)-6-(2-naphthyl)-3-piperidin-4-yl-3H- | | | | |
| imidazo[4,5-b]pyridine | | | | |
| 2-(5-methoxy-1H-indol-3-yl)-6-(2-naphthyl)-3-piperidin-4-yl-3H-imidazo[4,5- | | | | |
| b]pyridine | | | | |
| 2-(3-furyl)-6-(2-naphthyl)-3-piperidin-4-yl-3H-imidazo[4,5-b]pyridine | | | | |
| 5-[6-(2-naphthyl)-3-piperidin-4-yl-3H-imidazo[4,5-b]pyridin-2-yl]pyrimidine- | | | | |
| 2,4(1H,3H)-dione | | | | |
| 6-(2-naphthyl)-3-piperidin-4-yl-2-[3-(trifluoromethyl)phenyl]-3H-imidazo[4,5- | | | | |
| b]pyridine | | | | |
| 2-[2,5-bis(trifluoromethyl)phenyl]-6-(2-naphthyl)-3-piperidin-4-yl-3H-imidazo[4,5- | | | | |
| b]pyridine | | | | |
| 2-(4-fluorophenyl)-6-(2-naphthyl)-3-piperidin-4-yl-3H-imidazo[4,5-b]pyridine | | | | |
| N-{4-[6-(2-naphthyl)-3-piperidin-4-yl-3H-imidazo[4,5-b]pyridin-2- | | 1.460 | | |
| yl]phenyl}acetamide | | | | |
| 2-(1-benzothien-3-yl)-6-(2-naphthyl)-3-piperidin-4-yl-3H-imidazo[4,5-b]pyridine | | | | |
| 2-(5-methyl-1H-indol-3-yl)-6-(2-naphthyl)-3-piperidin-4-yl-3H-imidazo[4,5- | | | | |
| b]pyridine | | | | |
| 2-[6-(2-naphthyl)-3-piperidin-4-yl-3H-imidazo[4,5-b]pyridin-2-yl]quinoline | | | | |
| 7-(2-naphthyl)-3-piperidin-4-yl-1,3-dihydro-2H-imidazo[4,5-b]pyridin-2-one | | | 1.290 | 0.040 |
| 7-(2-naphthyl)-3-(2,2,6,6-tetramethylpiperidin-4-yl)-3H-imidazo[4,5-b]pyridine | | | | |
| 3-cyclohexyl-7-(2-naphthyl)-3H-imidazo[4,5-b]pyridine | | | | |
| 7-(2-naphthyl)-3-(tetrahydro-2H-pyran-4-yl)-3H-imidazo[4,5-b]pyridine | | | | |
| 2-(3-bromophenyl)-6-(2-naphthyl)-3-piperidin-4-yl-3H-imidazo[4,5-b]pyridine | | | | |
| 6-(2-naphthyl)-3-piperidin-4-yl-2-{3-[3-(trifluoromethyl)phenoxy]phenyl}-3H- | | | | |
| imidazo[4,5-b]pyridine | | | | |
| 6-(2-naphthyl)-3-piperidin-4-yl-2-(3-thienyl)-3H-imidazo[4,5-b]pyridine | | | | |
| 2-(4,5-dimethyl-2-furyl)-6-(2-naphthyl)-3-piperidin-4-yl-3H-imidazo[4,5-b]pyridine | | | | |
| tert-butyl 3-[2-methyl-7-(2-naphthyl)-3H-imidazo[4,5-b]pyridin-3-yl]azetidine-1- | | | | |
| carboxylate | | | | |
| tert-butyl (3R)-3-[2-methyl-7-(2-naphthyl)-3H-imidazo[4,5-b]pyridin-3- | | | | |
| yl]pyrrolidine-1-carboxylate | | | | |
| 1-methyl-7-(2-naphthyl)-3-piperidin-4-yl-1,3-dihydro-2H-imidazo[4,5-b]pyridin-2- | | | 1.070 | 0.000 |
| one | | | | |
| 2-[6-(2-naphthyl)-3-piperidin-4-yl-3H-imidazo[4,5-b]pyridin-2-yl]phenol | | 1.380 | | |
| 6-(2-naphthyl)-3-piperidin-4-yl-2-(3-vinylphenyl)-3H-imidazo[4,5-b]pyridine | | 1.290 | | |
| 2-(2-methoxy-1-naphthyl)-6-(2-naphthyl)-3-piperidin-4-yl-3H-imidazo[4,5-b]pyridine | | | | |
| 2-(2,3-difluorophenyl)-6-(2-naphthyl)-3-piperidin-4-yl-3H-imidazo[4,5-b]pyridine | | | | |
| 2-(4-chlorophenyl)-6-(2-naphthyl)-3-piperidin-4-yl-3H-imidazo[4,5-b]pyridine | | 1.160 | | |
| 6-(2-naphthyl)-2-(5-nitro-2-thienyl)-3-piperidin-4-yl-3H-imidazo[4,5-b]pyridine | | | | |
| 6-(2-naphthyl)-2-(1-oxidopyridin-4-yl)-3-piperidin-4-yl-3H-imidazo[4,5-b]pyridine | | | | |
| 7-(2-naphthyl)-3-piperidin-4-yl-1,3-dihydro-2H-imidazo[4,5-b]pyridine-2-thione | | | | |
| tert-butyl (3S)-3-[7-(2-naphthyl)-3H-imidazo[4,5-b]pyridin-3-yl]piperidine-1- | | | | |
| carboxylate | | | | |
| tert-butyl 4-{[7-(2-naphthyl)-3H-imidazo[4,5-b]pyridin-3-yl]methyl}piperidine-1- | | | | |
| carboxylate | | | | |
| tert-butyl (3S)-3-[2-methyl-7-(2-naphthyl)-3H-imidazo[4,5-b]pyridin-3-yl]pyrrolidine- | | | | |
| 1-carboxylate | | | | |
| 2,2,6,6-tetramethyl-4-[7-(2-naphthyl)-3H-imidazo[4,5-b]pyridin-3-yl]piperidine-1- | | | | |
| carbaldehyde | | | | |
| 2-methoxy-4-[6-(2-naphthyl)-3-piperidin-4-yl-3H-imidazo[4,5-b]pyridin-2-yl]phenol | 0.486 | 2.110 | | |
| 2-(2-bromophenyl)-6-(2-naphthyl)-3-piperidin-4-yl-3H-imidazo[4,5-b]pyridine | | | | |
| 2-(4-bromophenyl)-6-(2-naphthyl)-3-piperidin-4-yl-3H-imidazo[4,5-b]pyridine | | | | |
| 6-(2-naphthyl)-2-(3-phenoxyphenyl)-3-piperidin-4-yl-3H-imidazo[4,5-b]pyridine | | | | |
| 2-(3-fluorophenyl)-6-(2-naphthyl)-3-piperidin-4-yl-3H-imidazo[4,5-b]pyridine | | | | |
| 2-(1-methyl-1H-imidazol-2-yl)-6-(2-naphthyl)-3-piperidin-4-yl-3H-imidazo[4,5- | | 2.720 | | |
| b]pyridine | | | | |
| 2-(1-methyl-1H-pyrrol-2-yl)-6-(2-naphthyl)-3-piperidin-4-yl-3H-imidazo[4,5- | | 2.470 | | |
| b]pyridine | | | | |
| 2-(2-chloro-4-methyl-1-phenyl-1H-pyrrol-3-yl)-6-(2-naphthyl)-3-piperidin-4-yl-3H- | | | | |
| imidazo[4,5-b]pyridine | | | | |
| 3-[6-(2-naphthyl)-3-piperidin-4-yl-3H-imidazo[4,5-b]pyridin-2-yl]quinoline | | | | |
| 2-(3-ethylphenyl)-6-(2-naphthyl)-3-piperidin-4-yl-3H-imidazo[4,5-b]pyridine | | | | |

While particular embodiments of the present invention have been illustrated and described, it would be apparent to those skilled in the art that various other changes and modifications can be made without departing from the spirit and scope of the invention. It is therefore intended to cover in the appended claims all such changes and modifications that are within the scope of this invention.

What is claimed is:

1. A compound of Formula (A): 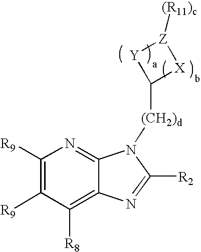
or a tautomer thereof or pharmaceutically acceptable salt thereof,
or Formula (B) 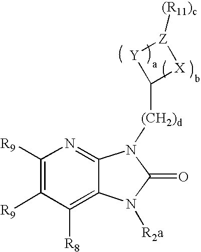
or a tautomer thereof or pharmaceutically acceptable salt thereof,
wherein
each R₁ is independently H, C₁-C₆ alkyl, C₂-C₆ alkenyl, C₂-C₆ alkynyl, C₁-C₆ alkyl carbonyl, C₃-C₈ cycloalkyl, fluorinated C₁-C₆ alkyl, CN, NO₂, halogen, COOR₃, OR₃, S(O)ₙR₃, NHC(O)C₁-C₆ alkyl, N(R₄)(R₅), SO₂N(R₄)(R₅), aryl, heteroaryl, or 3- to 7-membered heterocyclyl, wherein C₁-C₆ alkyl, C₂-C₆ alkenyl, C₂-C₆ alkynyl, C₁-C₆ alkoxyl, aryl, heteroaryl, or 3- to 7-membered heterocyclyl are optionally substituted with one or more R₉ group;
R₂ is H; C₁-C₆ alkyl; C₃-C₈ cycloalkyl; C₂-C₆ alkenyl, C₂-C₆ alkynyl, CN, NO2, CO—C₁-C₆ alkyl, halogen, COOR₃, OR₃, S(O)ₙR₃, NHC(O)C₁-C₆ alkyl, N(R₄)(R₅), SO₂N(R₄)(R₅), SH, aryl optionally fused to a heterocyclic ring, heteroary, 3- to 7-membered heterocyclyl, or fused aryl heterocyclyl, wherein C₁-C₆ alkyl, C₂-C₆ alkenyl, C₂-C₆ alkynyl, aryl, heteroaryl, or 3- to 7-membered heterocyclyl, fused arylheterocycle, or OR₃ are optionally substituted with one or more R₁ group;
R₂ₐ is C₁-C₆ alkyl or C₃-C₈ cycloalkyl, wherein each R₂ₐ is optionally substituted with halogen, hydroxyl, or C₁-C₆ alkoxyl;
each R₃ is independently H, CF₃, C₁-C₆ alkyl, C₃-C₈ cycloalkyl, 3- to 7-membered heterocyclyl, aryl, membered heteroaryl; wherein all except H and CF₃ are optionally substituted with halogen, hydroxyl, or C₁-C₆ alkoxyl;
R₄ and R₅ are each independently H, aryl, heteroaryl, C₁-C₆ alkyl, C₃-C₈ cycloalkyl, 3- to 7-membered heterocyclyl, aryl-C₁-C₆ alkyl, heteroaryl-C₁-C₆ alkyl, C₃-C₈ cycloalkyl-C₁-C₆ alkyl, 3- to 7-membered-heterocyclyl-C₁-C₆ alkyl, 3- to 7-membered-heterocyclyl-C₁-C₆alkyl-aryl, 3- to 7-membered-heterocyclyl-alkoxyaryl, 3- to 7-membered heterocyclyl-C₁-C₆ alkyl-heteroaryl, 3- to 7-membered-heterocyclyl-C₁-C₆ alkoxy-heteroaryl, C₁-C₆ alkoxyaryl, C₁-C₆ alkylamine-aryl, 3- to 7-membered heterocyclyl-C₁-C₆ alkylamine-aryl, 3- to 7-membered heterocyclyl-C₁-C₆ alkylamine-heteroaryl; wherein all except H are optionally substituted with C₁-C₆ alkyl optionally substituted with halogen, hydroxyl, or C₁-C₆ alkoxyl; or R₄ and R₅ when taken together with the ring to which they are attached form a 3- to 8-membered heterocyclyl having 0, 1, or 2 additional heteroatoms and optionally substituted with C₁-C₆ alkyl optionally substituted with halogen, hydroxyl, or C₁-C₆ alkoxyl;
R₆ and R₇ are each independently H, NH₂, CN, NO₂, C₁-C₆ alkyl, C₂-C₆ alkenyl, C₂-C₆ alkynyl, COOH, COOR₃, halogen, C₁-C₆ alkoxy, C₃-C₈ cycloalkyl, CF₃, S(O)ₙR₃ or OR₃; or R₆ and R₇ when taken together with the ring to which they are attached form C₅-C₇ cycloalkyl or C₆ aryl, optionally substituted with one or more R₁ groups;
R₈ is H or naphthyl optionally substituted with one or more R₁ groups;
each R₉ is independently H or C₆-C₁₀ aryl;
each R₁₁ is independently H, C₁-C₆ alkyl, C₂-C₆ alkenyl, C₂-C₆ alkynyl, C₁-C₆ alkyl carbonyl, C₃-C₈ cycloalkyl, fluorinated C₁-C₆ alkyl, CF₃, CN, NO₂, NH₂, halogen, COOR₃, OR₃, S(O)ₙR₃, NHC(O)C₁-C₆ alkyl, N(R₄)(R₅), SO₂N(R₄)(R₅), C(O)NR₄R₅, aryl, heteroaryl, and 3- to 7-membered heterocyclyl, wherein aryl, heteroaryl, heterocyclyl, C₁-C₆ alkyl, C₂-C₆ alkenyl, C₂-C₆ alkynyl, C₁-C₆ carbonyl, or cycloalkyl are optionally substituted with aryl, C₁-C₆ alkyl optionally substituted with halogen; halogen; hydroxyl; or C₁-C₆ alkoxy;
X is CR₆R₆ or CHR₆CHR₇;
Y is CR₁R₇;
Z is CH, N or O, with the proviso that when Z is O, c is zero;
a is 1, 2, 3 or 4;
b is 0, 1 or 2;
c is 0, 1 or 2;
d is 0, 1, 2 or 3; and
n is 0, 1 or 2.

2. The compound of claim 1, wherein Z is N.

3. The compound of claim 1, wherein a is 1, 2 or 3.

4. The compound of claim 1, wherein b is 1 or 2.

5. The compound of claim 1, wherein the ring containing X and Y is piperidinyl, pyrrolidinyl or azetidinyl, each being optionally independently substituted as defined in claim 1.

6. The compound of claim 1, wherein R₁, R₆ and R₇ are each independently H or C₁-C₆ alkyl, R₁ being optionally substituted as defined in claim 1.

7. The compound of claim 1, wherein each R₉ is independently H or naphthyl.

8. The compound of claim 1, wherein R₈ is H or naphthyl.

9. The compound of claim 1, wherein d is 0.

10. The compound of claim 1, wherein, R₂ is H; C₁-C₆ alkyl; C₃-C₈ cycloalkyl; 5- to 10-membered hetroaryl optionally substituted with one, two or three substituents independently selected from halogen, CN, OH, OC₁-C₆ alkyl optionally substituted with halogen, OC₆-C₁₀ aryl, C₁-C₆ alkyl optionally substituted with halogen, C₂-C₆ alkenyl, NO2, heterocyclyl, NHC(O)C₁-C₆ alkyl, NR₄R₅; C₆-C₁₀ aryl or CO—C₁-C₆ alkyl; heterocyclyl optionally substituted with one, two or three COOC₁-C₆ alkyl; C₆-C₁₀ aryl optionally fused to a heterocyclic ring or optionally substituted with one, two or three substituents independently selected from halogen, CN, OH, OC₁-C₆ alkyl optionally substituted with halogen, OC₆-C₁₀ aryl, C₁-C₆ alkyl optionally substituted with halogen, C₂-C₆ alkenyl, NO2, heterocyclyl, NHC(O)C₁-C₆ alkyl, NR₄R₅; OH; or SH.

11. The compound of claim 10, wherein the C₁-C₆ alkyl is methyl, ethyl, propyl, i-propyl, butyl, s-butyl, t-butyl or i-butyl.

12. The compound of claim 10, wherein the C₃-C₈ cycloalkyl is cyclohexyl.

13. The compound of claim 10, wherein the 5- to 10-membered heteroaryl is pyridyl, N-oxo pyridyl, pyrrolyl, indolyl, imidazolyl, thiophenyl, benzothiophenyl, quinolinyl, furanyl, or benzofuranyl, each being optionally independently substituted as defined in claim 10.

14. The compound of claim 10, wherein the heterocyclyl is piperidinyl or pyrimidinedion-yl, each being optionally independently substituted as defined in claim 10.

15. The compound of claim 10, wherein the C₆-C₁₀ aryl is phenyl or naphthyl, each being optionally independently substituted as defined in claim 10.

16. The compound of claim 1, wherein R₁₁ is H; COOR₃; C₁-C₆ alkyl carbonyl; CONR₄R₅; SO₂NR₄R₅; SO₂R₃; C₁-C₆ alkyl optionally substituted with one or two substituents independently selected from the group consisting of aryl and halogen; or NR₄R₅.

17. The compound of claim 16, wherein COOR₃ is COO—C₁-C₆ alkyl.

18. The compound of claim 16, wherein SO₂R₃ is SO₂aryl or SO₂C₁-C₆ alkyl.

19. The compound of claim 1, wherein the compound of Formula (A) is a compound of Formula (A₂): 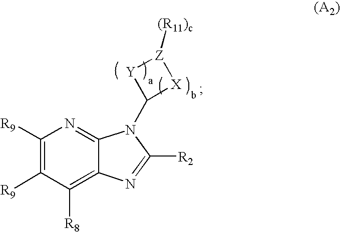
wherein R₂, R₈, R₉, R₁₁, X, Y, a, b, c and d are defined as in claim 1.

20. The compound of claim 1, wherein the compound is one of the following:
7-(2-naphthyl)-3-piperidin-4-yl-2-pyridin-2-yl-3H-imidazo[4,5-b]pyridine;
7-(2-naphthyl)-3-piperidin-4-yl-2-pyridin-3-yl-3H-imidazo[4,5-b]pyridine;
7-(2-naphthyl)-3-piperidin-4-yl-2-pyridin-4-yl-3H-imidazo[4,5-b]pyridine;
7-(2-naphthyl)-3-piperidin-4-yl-2-(1H-pyrrol-2-yl)-3H-imidazo[4,5-b]pyridine;
2-(1-methyl-1H-indol-2-yl)-7-(2-naphthyl)-3-piperidin-4-yl-3H-imidazo[4,5-b]pyridine;
7-(2-naphthyl)-3-piperidin-4-yl-2-(2-thienyl)-3H-imidazo[4,5-b]pyridine;
2-(4-morpholin-4-yl-3-nitrophenyl)-7-(2-naphthyl)-3-piperidin-4-yl-3H-imidazo[4,5-b]pyridine;
8-[7-(2-naphthyl)-3-piperidin-4-yl-3H-imidazo[4,5-b]pyridin-2-yl]quinoline;
5-[7-(2-naphthyl)-3-piperidin-4-yl-3H-imidazo[4,5-b]pyridin-2-yl]quinoline;
2-(1-benzofuran-5-yl)-7-(2-naphthyl)-3-piperidin-4-yl-3H-imidazo[4,5-b]pyridine;
2-(5-methyl-2-furyl)-7-(2-naphthyl)-3-piperidin-4-yl-3H-imidazo[4,5-b]pyridine;
2-(1-methyl-1H-indol-3-yl)-7-(2-naphthyl)-3-piperidin-4-yl-3H-imidazo[4,5-b]pyridine;
2-(2-methoxypyridin-3-yl)-7-(2-naphthyl)-3-piperidin-4-yl-3H-imidazo[4,5-b]pyridine;
4-[7-(2-naphthyl)-3-piperidin-4-yl-3H-imidazo[4,5-b]pyridin-2-yl]quinoline;
2-(2-furyl)-7-(2-naphthyl)-3-piperidin-4-yl-3H-imidazo[4,5-b]pyridine;
2-(1-benzofuran-2-yl)-7-(2-naphthyl)-3-piperidin-4-yl-3H-imidazo[4,5-b]pyridine;
2-(1-benzothien-2-yl)-7-(2-naphthyl)-3-piperidin-4-yl-3H-imidazo[4,5-b]pyridine;
2-isobutyl-7-(2-naphthyl)-3-piperidin-4-yl-3H-imidazo[4,5-b]pyridine;
2-cyclohexyl-7-(2-naphthyl)-3-piperidin-4-yl-3H-imidazo[4,5-b]pyridine;
7-(2-naphthyl)-3-piperidin-4-yl-2-propyl-3H-imidazo[4,5-b]pyridine;
2-butyl-7-(2-naphthyl)-3-piperidin-4-yl-3H-imidazo[4,5-b]pyridine;
2-ethyl-7-(2-naphthyl)-3-piperidin-4-yl-3H-imidazo[4,5-b]pyridine;
2-(4-isopropylphenyl)-7-(2-naphthyl)-3-piperidin-4-yl-3H-imidazo[4,5-b]pyridine;
2-(4-tert-butylphenyl)-7-(2-naphthyl)-3-piperidin-4-yl-3H-imidazo[4,5-b]pyridine;
2-(3-methoxyphenyl)-7-(2-naphthyl)-3-piperidin-4-yl-3H-imidazo[4,5-b]pyridine;
7-(2-naphthyl)-2-(2-nitrophenyl)-3-piperidin-4-yl-3H-imidazo[4,5-b]pyridine;
2-(4-methylphenyl)-7-(2-naphthyl)-3-piperidin-4-yl-3H-imidazo[4,5-b]pyridine;
7-(2-naphthyl)-2-(3-nitrophenyl)-3-piperidin-4-yl-3H-imidazo[4,5-b]pyridine;
7-(2-naphthyl)-2-phenyl-3-piperidin-4-yl-3H-imidazo[4,5-b]pyridine;
2-(2-methoxyphenyl)-7-(2-naphthyl)-3-piperidin-4-yl-3H-imidazo[4,5-b]pyridine;
2-(3-methylphenyl)-7-(2-naphthyl)-3-piperidin-4-yl-3H-imidazo[4,5-b]pyridine;
2-(2,4-dimethylphenyl)-7-(2-naphthyl)-3-piperidin-4-yl-3H-imidazo[4,5-b]pyridine;
2-(2,4-dichlorophenyl)-7-(2-naphthyl)-3-piperidin-4-yl-3H-imidazo[4,5-b]pyridine;
2-(1-naphthyl)-7-(2-naphthyl)-3-piperidin-4-yl-3H-imidazo[4,5-b]pyridine;
2,7-di-2-naphthyl-3-piperidin-4-yl-3H-imidazo[4,5-b]pyridine;
2-(2,4-difluorophenyl)-7-(2-naphthyl)-3-piperidin-4-yl-3H-imidazo[4,5-b]pyridine;
2-(3-chlorophenyl)-7-(2-naphthyl)-3-piperidin-4-yl-3H-imidazo[4,5-b]pyridine;
2-(2-fluorophenyl)-7-(2-naphthyl)-3-piperidin-4-yl-3H-imidazo[4,5-b]pyridine;
4-[7-(2-naphthyl)-3-piperidin-4-yl-3H-imidazo[4,5-b]pyridin-2-yl]phenol;
3-[7-(2-naphthyl)-3-piperidin-4-yl-3H-imidazo[4,5-b]pyridin-2-yl]phenol;
2-[7-(2-naphthyl)-3-piperidin-4-yl-3H-imidazo[4,5-b]pyridin-2-yl]phenol;
3-[7-(2-naphthyl)-3-piperidin-4-yl-3H-imidazo[4,5-b]pyridin-2-yl]benzene-1,2-diol;
2-methoxy-4-[7-(2-naphthyl)-3-piperidin-4-yl-3H-imidazo[4,5-b]pyridin-2-yl]phenol;
3-[7-(2-naphthyl)-3-piperidin-4-yl-3H-imidazo[4,5-b]pyridin-2-yl]-4-nitrophenol;
2-(4-methoxyphenyl)-7-(2-naphthyl)-3-piperidin-4-yl-3H-imidazo[4,5-b]pyridine;
2-(2-bromophenyl)-7-(2-naphthyl)-3-piperidin-4-yl-3H-imidazo[4,5-b]pyridine;
2-(3-bromophenyl)-7-(2-naphthyl)-3-piperidin-4-yl-3H-imidazo[4,5-b]pyridine;
2-(4-bromophenyl)-7-(2-naphthyl)-3-piperidin-4-yl-3H-imidazo[4,5-b]pyridine;
7-(2-naphthyl)-3-piperidin-4-yl-2-(3-vinylphenyl)-3H-imidazo[4,5-b]pyridine;
4-[7-(2-naphthyl)-3-piperidin-4-yl-3H-imidazo[4,5-b]pyridin-2-yl]-N,N-diphenylaniline;
2-(2-methoxy-1-naphthyl)-7-(2-naphthyl)-3-piperidin-4-yl-3H-imidazo[4,5-b]pyridine;
7-(2-naphthyl)-2-(3-phenoxyphenyl)-3-piperidin-4-yl-3H-imidazo[4,5-b]pyridine;
7-(2-naphthyl)-3-piperidin-4-yl-2-{3-[3-(trifluoromethyl)phenoxy]phenyl}-3H-imidazo[4,5-b]pyridine;
7-(2-naphthyl)-3-piperidin-4-yl-2-[4-(trifluoromethyl)phenyl]-3H-imidazo[4,5-b]pyridine;
7-(2-naphthyl)-3-piperidin-4-yl-2-[3-(trifluoromethyl)phenyl]-3H-imidazo[4,5-b]pyridine;
7-(2-naphthyl)-3-piperidin-4-yl-2-[2-(trifluoromethyl)phenyl]-3H-imidazo[4,5-b]pyridine;
2-(2,6-difluorophenyl)-7-(2-naphthyl)-3-piperidin-4-yl-3H-imidazo[4,5-b]pyridine;
2-(2,3-difluorophenyl)-7-(2-naphthyl)-3-piperidin-4-yl-3H-imidazo[4,5-b]pyridine;
7-(2-naphthyl)-3-piperidin-4-yl-2-[4-(trifluoromethoxy)phenyl]-3H-imidazo[4,5-b]pyridine;
7-(2-naphthyl)-3-piperidin-4-yl-2-[2-(trifluoromethoxy)phenyl]-3H-imidazo[4,5-b]pyridine;
2-[2,5-bis(trifluoromethyl)phenyl]-7-(2-naphthyl)-3-piperidin-4-yl-3H-imidazo[4,5-b]pyridine;
2-[4-fluoro-2-(trifluoromethyl)phenyl]-7-(2-naphthyl)-3-piperidin-4-yl-3H-imidazo[4,5-b]pyridine;
2-[2-chloro-3-(trifluoromethyl)phenyl]-7-(2-naphthyl)-3-piperidin-4-yl-3H-imidazo[4,5-b]pyridine;
2-(3-fluorophenyl)-7-(2-naphthyl)-3-piperidin-4-yl-3H-imidazo[4,5-b]pyridine;
2-(2-chlorophenyl)-7-(2-naphthyl)-3-piperidin-4-yl-3H-imidazo[4,5-b]pyridine;
2-(4-fluorophenyl)-7-(2-naphthyl)-3-piperidin-4-yl-3H-imidazo[4,5-b]pyridine;
4-[7-(2-naphthyl)-3-piperidin-4-yl-3H-imidazo[4,5-b]pyridin-2-yl]benzonitrile;
N-{4-[7-(2-naphthyl)-3-piperidin-4-yl-3H-imidazo[4,5-b]pyridin-2-yl]phenyl}acetamide;
7-(2-naphthyl)-2-(4-nitrophenyl)-3-piperidin-4-yl-3H-imidazo[4,5-b]pyridine;
2-(4-chlorophenyl)-7-(2-naphthyl)-3-piperidin-4-yl-3H-imidazo[4,5-b]pyridine;
2-(1-ethyl-5-methyl-1H-imidazol-4-yl)-7-(2-naphthyl)-3-piperidin-4-yl-3H-imidazo[4,5-b]pyridine;
2-(1-methyl-1H-imidazol-2-yl)-7-(2-naphthyl)-3-piperidin-4-yl-3H-imidazo[4,5-b]pyridine;
2-(4-methyl-1H-imidazol-5-yl)-7-(2-naphthyl)-3-piperidin-4-yl-3H-imidazo[4,5-b]pyridine;
2-(1H-imidazol-2-yl)-7-(2-naphthyl)-3-piperidin-4-yl-3H-imidazo[4,5-b]pyridine;
2-(2-methyl-1H-imidazol-4-yl)-7-(2-naphthyl)-3-piperidin-4-yl-3H-imidazo[4,5-b]pyridine;
7-(2-naphthyl)-2-(5-nitro-2-thienyl)-3-piperidin-4-yl-3H-imidazo[4,5-b]pyridine;
7-(2-naphthyl)-3-piperidin-4-yl-2-(3-thienyl)-3H-imidazo[4,5-b]pyridine;
2-(1-benzothien-3-yl)-7-(2-naphthyl)-3-piperidin-4-yl-3H-imidazo[4,5-b]pyridine;
2-(5-methoxy-1H-indol-3-yl)-7-(2-naphthyl)-3-piperidin-4-yl-3H-imidazo[4,5-b]pyridine;
2-(5-methyl-1H-indol-3-yl)-7-(2-naphthyl)-3-piperidin-4-yl-3H-imidazo[4,5-b]pyridine;
2-(1-acetyl-1H-indol-3-yl)-7-(2-naphthyl)-3-piperidin-4-yl-3H-imidazo[4,5-b]pyridine;
2-(4,5-dimethyl-2-furyl)-7-(2-naphthyl)-3-piperidin-4-yl-3H-imidazo[4,5-b]pyridine;
7-(2-naphthyl)-2-(1-oxidopyridin-4-yl)-3-piperidin-4-yl-3H-imidazo[4,5-b]pyridine;
2-(3,5-dichloropyridin-4-yl)-7-(2-naphthyl)-3-piperidin-4-yl-3H-imidazo[4,5-b]pyridine;
2-(1-methyl-1H-pyrrol-2-yl)-7-(2-naphthyl)-3-piperidin-4-yl-3H-imidazo[4,5-b]pyridine;
5-[7-(2-naphthyl)-3-piperidin-4-yl-3H-imidazo[4,5-b]pyridin-2-yl]pyrimidine-2,4(1H,3H)-dione;
2-(1,3-benzodioxol-5-yl)-7-(2-naphthyl)-3-piperidin-4-yl-3H-imidazo[4,5-b]pyridine;
2-(2,3-dihydro-1,4-benzodioxin-6-yl)-7-(2-naphthyl)-3-piperidin-4-yl-3H-imidazo[4,5-b]pyridine;
2-(2-chloro-4-methyl-1-phenyl-1H-pyrrol-3-yl)-7-(2-naphthyl)-3-piperidin-4-yl-3H-imidazo[4,5-b]pyridine;
2-chloro-3-[7-(2-naphthyl)-3-piperidin-4-yl-3H-imidazo[4,5-b]pyridin-2-yl]quinoline;
3-[7-(2-naphthyl)-3-piperidin-4-yl-3H-imidazo[4,5-b]pyridin-2-yl]quinoline;
2-[7-(2-naphthyl)-3-piperidin-4-yl-3H-imidazo[4,5-b]pyridin-2-yl]quinoline;
2-cyclopentyl-7-(2-naphthyl)-3-piperidin-4-yl-3H-imidazo[4,5-b]pyridine;
7-(2-naphthyl)-3-piperidin-4-yl-2-[3-(trifluoromethoxy)phenyl]-3H-imidazo[4,5-b]pyridine;
7-(2-naphthyl)-3-piperidin-4-yl-3H-imidazo[4,5-b]pyridine;
2-methyl-7-(2-naphthyl)-3-piperidin-4-yl-3H-imidazo[4,5-b]pyridine;
7-(2-naphthyl)-3-[1-(trifluoroacetyl)piperidin-4-yl]-3H-imidazo[4,5-b]pyridine;
methyl 4-[7-(2-naphthyl)-3H-imidazo[4,5-b]pyridin-3-yl]piperidine-1-carboxylate;
N,N-dimethyl-4-[7-(2-naphthyl)-3H-imidazo[4,5-b]pyridin-3-yl]piperidine-1-carboxamide;
N-ethyl-4-[7-(2-naphthyl)-3H-imidazo[4,5-b]pyridin-3-yl]piperidine-1-carboxamide;
N-isopropyl-4-[7-(2-naphthyl)-3H-imidazo[4,5-b]pyridin-3-yl]piperidine-1-carboxamide;
3-(1-benzylpiperidin-4-yl)-7-(2-naphthyl)-3H-imidazo[4,5-b]pyridine;
3-[1-(methylsulfonyl)piperidin-4-yl]-7-(2-naphthyl)-3H-imidazo[4,5-b]pyridine;
N-cyclohexyl-4-[7-(2-naphthyl)-3H-imidazo[4,5-b]pyridin-3-yl]piperidine-1-carboxamide;
3-[1-(morpholin-4-ylcarbonyl)piperidin-4-yl]-7-(2-naphthyl)-3H-imidazo[4,5-b]pyridine;
N-(3′,6′-dihydroxy-3-oxo-3H-spiro[2-benzofuran-1,9′-xanthen]-5-yl)-4-[7-(2-naphthyl)-3H-imidazo[4,5-b]pyridin-3-yl]piperidine-1-carbothioamide;
N,N-dimethyl-4-[7-(2-naphthyl)-3H-imidazo[4,5-b]pyridin-3-yl]piperidine-1-sulfonamide;
7-(2-naphthyl)-3-(piperidin-4-ylmethyl)-3H-imidazo[4,5-b]pyridine;
3-[3-(1H-imidazol-1-yl)propyl]-7-(2-naphthyl)-3H-imidazo[4,5-b]pyridine;
2-methyl-7-(2-naphthyl)-3-[(3R)-pyrrolidin-3-yl]-3H-imidazo[4,5-b]pyridine;
2-methyl-3-[2-(4-methylphenyl)ethyl]-7-(2-naphthyl)-3H-imidazo[4,5-b]pyridine;
7-(2-naphthyl)-3-[(3S)-piperidin-3-yl]-3H-imidazo[4,5-b]pyridine;
3-[2-(4-methylphenyl)ethyl]-7-(2-naphthyl)-3H-imidazo[4,5-b]pyridine;
2-methyl-7-(2-naphthyl)-3-(piperidin-4-ylmethyl)-3H-imidazo[4,5-b]pyridine;
3-azetidin-3-yl-7-(2-naphthyl)-3H-imidazo[4,5-b]pyridine;
7-(2-naphthyl)-3-[(3R)-pyrrolidin-3-yl]-3H-imidazo[4,5-b]pyridine;
7-(2-naphthyl)-3-[(3S)-pyrrolidin-3-yl]-3H-imidazo[4,5-b]pyridine;
N-isopropyl-4-[7-(2-naphthyl)-2-oxo-1,2-dihydro-3H-imidazo[4,5-b]pyridin-3-yl]piperidine-1-carboxamide;
3-(1-acetylpiperidin-4-yl)-1-methyl-7-(2-naphthyl)-1,3-dihydro-2H-imidazo[4,5-b]pyridin-2-one;
N,N-dimethyl-4-[1-methyl-7-(2-naphthyl)-2-oxo-1,2-dihydro-3H-imidazo[4,5-b]pyridin-3-yl]piperidine-1-carboxamide;
2-methyl-7-(2-naphthyl)-3-[(3S)-pyrrolidin-3-yl]-3H-imidazo[4,5-b]pyridine;
2-methyl-7-(2-naphthyl)-3-[(3R)-piperidin-3-yl]-3H-imidazo[4,5-b]pyridine;
3-[1-(morpholin-4-ylcarbonyl)piperidin-4-yl]-7-(2-naphthyl)-1,3-dihydro-2H-imidazo[4,5-b]pyridin-2-one;
7-(2-naphthyl)-3-[(3R)-piperidin-3-yl]-3H-imidazo[4,5-b]pyridine;
3-azetidin-3-yl-2-methyl-7-(2-naphthyl)-3H-imidazo[4,5-b]pyridine;
2-methyl-7-(2-naphthyl)-3-[(3S)-piperidin-3-yl]-3H-imidazo[4,5-b]pyridine;
N-isopropyl-4-[1-methyl-7-(2-naphthyl)-2-oxo-1,2-dihydro-3H-imidazo[4,5-b]pyridin-3-yl]piperidine-1-carboxamide;
6-(2-naphthyl)-3-piperidin-4-yl-2-(1H-pyrrol-2-yl)-3H-imidazo[4,5-b]pyridine;
2-(1-methyl-1H-indol-2-yl)-6-(2-naphthyl)-3-piperidin-4-yl-3H-imidazo[4,5-b]pyridine;
2-(1H-indol-3-yl)-6-(2-naphthyl)-3-piperidin-4-yl-3H-imidazo[4,5-b]pyridine;
6-(2-naphthyl)-3-piperidin-4-yl-3H-imidazo[4,5-b]pyridine;
2-methyl-6-(2-naphthyl)-3-piperidin-4-yl-3H-imidazo[4,5-b]pyridine;
4-[6-(2-naphthyl)-3-piperidin-4-yl-3H-imidazo[4,5-b]pyridin-2-yl]quinoline;
2-(4-isopropylphenyl)-6-(2-naphthyl)-3-piperidin-4-yl-3H-imidazo[4,5-b]pyridine;
2-(2,4-dichlorophenyl)-6-(2-naphthyl)-3-piperidin-4-yl-3H-imidazo[4,5-b]pyridine;
2-(3-chlorophenyl)-6-(2-naphthyl)-3-piperidin-4-yl-3H-imidazo[4,5-b]pyridine;
6-(2-naphthyl)-3-piperidin-4-yl-2-pyridin-4-yl-3H-imidazo[4,5-b]pyridine;
6-(2-naphthyl)-3-piperidin-4-yl-2-(2-thienyl)-3H-imidazo[4,5-b]pyridine;
2-(2-methoxypyridin-3-yl)-6-(2-naphthyl)-3-piperidin-4-yl-3H-imidazo[4,5-b]pyridine;
6-(2-naphthyl)-2-(3-nitrophenyl)-3-piperidin-4-yl-3H-imidazo[4,5-b]pyridine;
2-ethyl-6-(2-naphthyl)-3-piperidin-4-yl-3H-imidazo[4,5-b]pyridine;
1-methyl-3-[1-(morpholin-4-ylcarbonyl)piperidin-4-yl]-7-(2-naphthyl)-1,3-dihydro-2H-imidazo[4,5-b]pyridin-2-one;
1-methyl-3-[1-(methylsulfonyl)piperidin-4-yl]-7-(2-naphthyl)-1,3-dihydro-2H-imidazo[4,5-b]pyridin-2-one;
6-(2-naphthyl)-3-piperidin-4-yl-2-pyridin-2-yl-3H-imidazo[4,5-b]pyridine;
2-(3-methoxyphenyl)-6-(2-naphthyl)-3-piperidin-4-yl-3H-imidazo[4,5-b]pyridine;
2-(4-methylphenyl)-6-(2-naphthyl)-3-piperidin-4-yl-3H-imidazo[4,5-b]pyridine;
4-[6-(2-naphthyl)-3-piperidin-4-yl-3H-imidazo[4,5-b]pyridin-2-yl]phenol;
2-butyl-6-(2-naphthyl)-3-piperidin-4-yl-3H-imidazo[4,5-b]pyridine;
2-(2-fluorophenyl)-6-(2-naphthyl)-3-piperidin-4-yl-3H-imidazo[4,5-b]pyridine;
2-isopropyl-6-(2-naphthyl)-3-piperidin-4-yl-3H-imidazo[4,5-b]pyridine;
2-(2-furyl)-6-(2-naphthyl)-3-piperidin-4-yl-3H-imidazo[4,5-b]pyridine;
2-(1-benzothien-2-yl)-6-(2-naphthyl)-3-piperidin-4-yl-3H-imidazo[4,5-b]pyridine;
2-cyclohexyl-6-(2-naphthyl)-3-piperidin-4-yl-3H-imidazo[4,5-b]pyridine;
6-(2-naphthyl)-3-piperidin-4-yl-2-propyl-3H-imidazo[4,5-b]pyridine;
2-(2-methoxyphenyl)-6-(2-naphthyl)-3-piperidin-4-yl-3H-imidazo[4,5-b]pyridine;
2-(2,4-difluorophenyl)-6-(2-naphthyl)-3-piperidin-4-yl-3H-imidazo[4,5-b]pyridine;
2-(1-benzofuran-5-yl)-6-(2-naphthyl)-3-piperidin-4-yl-3H-imidazo[4,5-b]pyridine;
2-(5-methyl-2-furyl)-6-(2-naphthyl)-3-piperidin-4-yl-3H-imidazo[4,5-b]pyridine;
2-(1-benzofuran-2-yl)-6-(2-naphthyl)-3-piperidin-4-yl-3H-imidazo[4,5-b]pyridine;
2-(4-tert-butylphenyl)-6-(2-naphthyl)-3-piperidin-4-yl-3H-imidazo[4,5-b]pyridine;
2-(2,4-dimethylphenyl)-6-(2-naphthyl)-3-piperidin-4-yl-3H-imidazo[4,5-b]pyridine;
2-(1-naphthyl)-6-(2-naphthyl)-3-piperidin-4-yl-3H-imidazo[4,5-b]pyridine;
3-[6-(2-naphthyl)-3-piperidin-4-yl-3H-imidazo[4,5-b]pyridin-2-yl]phenol;
6-(2-naphthyl)-3-piperidin-4-yl-2-pyridin-3-yl-3H-imidazo[4,5-b]pyridine;
2-(4-morpholin-4-yl-3-nitrophenyl)-6-(2-naphthyl)-3-piperidin-4-yl-3H-imidazo[4,5-b]pyridine;
8-[6-(2-naphthyl)-3-piperidin-4-yl-3H-imidazo[4,5-b]pyridin-2-yl]quinoline;
5-[6-(2-naphthyl)-3-piperidin-4-yl-3H-imidazo[4,5-b]pyridin-2-yl]quinoline;
2-(1-methyl-1H-indol-3-yl)-6-(2-naphthyl)-3-piperidin-4-yl-3H-imidazo[4,5-b]pyridine;
2-isobutyl-6-(2-naphthyl)-3-piperidin-4-yl-3H-imidazo[4,5-b]pyridine;
6-(2-naphthyl)-2-(2-nitrophenyl)-3-piperidin-4-yl-3H-imidazo[4,5-b]pyridine;
6-(2-naphthyl)-2-phenyl-3-piperidin-4-yl-3H-imidazo[4,5-b]pyridine;
2-(3-methylphenyl)-6-(2-naphthyl)-3-piperidin-4-yl-3H-imidazo[4,5-b]pyridine;
2,6-di-2-naphthyl-3-piperidin-4-yl-3H-imidazo[4,5-b]pyridine;
tert-butyl (3R)-3-[7-(2-naphthyl)-3H-imidazo[4,5-b]pyridin-3-yl]pyrrolidine-1-carboxylate;
tert-butyl (3S)-3-[7-(2-naphthyl)-3H-imidazo[4,5-b]pyridin-3-yl]pyrrolidine-1-carboxylate;
6-(2-naphthyl)-3-piperidin-4-yl-2-[2-(trifluoromethyl)phenyl]-3H-imidazo[4,5-b]pyridine;
6-(2-naphthyl)-3-piperidin-4-yl-2-[4-(trifluoromethoxy)phenyl]-3H-imidazo[4,5-b]pyridine;
4-[6-(2-naphthyl)-3-piperidin-4-yl-3H-imidazo[4,5-b]pyridin-2-yl]benzonitrile;
2-(1H-imidazol-2-yl)-6-(2-naphthyl)-3-piperidin-4-yl-3H-imidazo[4,5-b]pyridine;
2-(3,5-dichloropyridin-4-yl)-6-(2-naphthyl)-3-piperidin-4-yl-3H-imidazo[4,5-b]pyridine;
2-(1,3-benzodioxol-5-yl)-6-(2-naphthyl)-3-piperidin-4-yl-3H-imidazo[4,5-b]pyridine;
tert-butyl (3S)-3-[2-methyl-7-(2-naphthyl)-3H-imidazo[4,5-b]pyridin-3-yl]piperidine-1-carboxylate;
5-(2-naphthyl)-3-piperidin-4-yl-3H-imidazo[4,5-b]pyridine;
4-[6-(2-naphthyl)-3-piperidin-4-yl-3H-imidazo[4,5-b]pyridin-2-yl]-N,N-diphenylaniline;
6-(2-naphthyl)-3-piperidin-4-yl-2-[4-(trifluoromethyl)phenyl]-3H-imidazo[4,5-b]pyridine;
2-[2-chloro-3-(trifluoromethyl)phenyl]-6-(2-naphthyl)-3-piperidin-4-yl-3H-imidazo[4,5-b]pyridine;
6-(2-naphthyl)-2-(4-nitrophenyl)-3-piperidin-4-yl-3H-imidazo[4,5-b]pyridine;
2-(2-methyl-1H-imidazol-4-yl)-6-(2-naphthyl)-3-piperidin-4-yl-3H-imidazo[4,5-b]pyridine;
6-(2-naphthyl)-3-piperidin-4-yl-2-[3-(trifluoromethoxy)phenyl]-3H-imidazo[4,5-b]pyridine;
tert-butyl 3-[7-(2-naphthyl)-3H-imidazo[4,5-b]pyridin-3-yl]azetidine-1-carboxylate;
tert-butyl (3R)-3-[7-(2-naphthyl)-3H-imidazo[4,5-b]pyridin-3-yl]piperidine-1-carboxylate;
tert-butyl (3R)-3-[2-methyl-7-(2-naphthyl)-3H-imidazo[4,5-b]pyridin-3-yl]piperidine-1-carboxylate;
2-(4-methoxyphenyl)-6-(2-naphthyl)-3-piperidin-4-yl-3H-imidazo[4,5-b]pyridine;
6-(2-naphthyl)-3-piperidin-4-yl-2-[2-(trifluoromethoxy)phenyl]-3H-imidazo[4,5-b]pyridine;
2-(2-chlorophenyl)-6-(2-naphthyl)-3-piperidin-4-yl-3H-imidazo[4,5-b]pyridine;
2-(4-methyl-1H-imidazol-5-yl)-6-(2-naphthyl)-3-piperidin-4-yl-3H-imidazo[4,5-b]pyridine;
2-(1-acetyl-1H-indol-3-yl)-6-(2-naphthyl)-3-piperidin-4-yl-3H-imidazo[4,5-b]pyridine;
2-(2,3-dihydro-1,4-benzodioxin-6-yl)-6-(2-naphthyl)-3-piperidin-4-yl-3H-imidazo[4,5-b]pyridine;
2-chloro-3-[6-(2-naphthyl)-3-piperidin-4-yl-3H-imidazo[4,5-b]pyridin-2-yl]quinoline;
7-(2-naphthyl)-3-piperidin-4-yl-3H-imidazo[4,5-b]pyridine;
2-methyl-7-(2-naphthyl)-3-piperidin-4-yl-3H-imidazo[4,5-b]pyridine;
3-[6-(2-naphthyl)-3-piperidin-4-yl-3H-imidazo[4,5-b]pyridin-2-yl]benzene-1,2-diol;
3-[6-(2-naphthyl)-3-piperidin-4-yl-3H-imidazo[4,5-b]pyridin-2-yl]-4-nitrophenol;
2-(2,6-difluorophenyl)-6-(2-naphthyl)-3-piperidin-4-yl-3H-imidazo[4,5-b]pyridine;
2-[4-fluoro-2-(trifluoromethyl)phenyl]-6-(2-naphthyl)-3-piperidin-4-yl-3H-imidazo[4,5-b]pyridine;
2-(1-ethyl-5-methyl-1H-imidazol-4-yl)-6-(2-naphthyl)-3-piperidin-4-yl-3H-imidazo[4,5-b]pyridine;
2-(5-methoxy-1H-indol-3-yl)-6-(2-naphthyl)-3-piperidin-4-yl-3H-imidazo[4,5-b]pyridine;
2-(3-furyl)-6-(2-naphthyl)-3-piperidin-4-yl-3H-imidazo[4,5-b]pyridine;
5-[6-(2-naphthyl)-3-piperidin-4-yl-3H-imidazo[4,5-b]pyridin-2-yl]pyrimidine-2,4(1H,3H)-dione;
6-(2-naphthyl)-3-piperidin-4-yl-2-[3-(trifluoromethyl)phenyl]-3H-imidazo[4,5-b]pyridine;
2-[2,5-bis(trifluoromethyl)phenyl]-6-(2-naphthyl)-3-piperidin-4-yl-3H-imidazo[4,5-b]pyridine;
2-(4-fluorophenyl)-6-(2-naphthyl)-3-piperidin-4-yl-3H-imidazo[4,5-b]pyridine;
N-{4-[6-(2-naphthyl)-3-piperidin-4-yl-3H-imidazo[4,5-b]pyridin-2-yl]phenyl}acetamide;
2-(1-benzothien-3-yl)-6-(2-naphthyl)-3-piperidin-4-yl-3H-imidazo[4,5-b]pyridine;
2-(5-methyl-1H-indol-3-yl)-6-(2-naphthyl)-3-piperidin-4-yl-3H-imidazo[4,5-b]pyridine;
2-[6-(2-naphthyl)-3-piperidin-4-yl-3H-imidazo[4,5-b]pyridin-2-yl]quinoline;
7-(2-naphthyl)-3-piperidin-4-yl-1,3-dihydro-2H-imidazo[4,5-b]pyridin-2-one;
7-(2-naphthyl)-3-(2,2,6,6-tetramethylpiperidin-4-yl)-3H-imidazo[4,5-b]pyridine;
3-cyclohexyl-7-(2-naphthyl)-3H-imidazo[4,5-b]pyridine;
7-(2-naphthyl)-3-(tetrahydro-2H-pyran-4-yl)-3H-imidazo[4,5-b]pyridine;
2-(3-bromophenyl)-6-(2-naphthyl)-3-piperidin-4-yl-3H-imidazo[4,5-b]pyridine;
6-(2-naphthyl)-3-piperidin-4-yl-2-{3-[3-(trifluoromethyl)phenoxy]phenyl}-3H-imidazo[4,5-b]pyridine;
6-(2-naphthyl)-3-piperidin-4-yl-2-(3-thienyl)-3H-imidazo[4,5-b]pyridine;
2-(4,5-dimethyl-2-furyl)-6-(2-naphthyl)-3-piperidin-4-yl-3H-imidazo[4,5-b]pyridine;
tert-butyl 3-[2-methyl-7-(2-naphthyl)-3H-imidazo[4,5-b]pyridin-3-yl]azetidine-1-carboxylate;
tert-butyl (3R)-3-[2-methyl-7-(2-naphthyl)-3H-imidazo[4,5-b]pyridin-3-yl]pyrrolidine-1-carboxylate;
1-methyl-7-(2-naphthyl)-3-piperidin-4-yl-1,3-dihydro-2H-imidazo[4,5-b]pyridin-2-one;
2-[6-(2-naphthyl)-3-piperidin-4-yl-3H-imidazo[4,5-b]pyridin-2-yl]phenol;
6-(2-naphthyl)-3-piperidin-4-yl-2-(3-vinylphenyl)-3H-imidazo[4,5-b]pyridine;
2-(2-methoxy-1-naphthyl)-6-(2-naphthyl)-3-piperidin-4-yl-3H-imidazo[4,5-b]pyridine;
2-(2,3-difluorophenyl)-6-(2-naphthyl)-3-piperidin-4-yl-3H-imidazo[4,5-b]pyridine;
2-(4-chlorophenyl)-6-(2-naphthyl)-3-piperidin-4-yl-3H-imidazo[4,5-b]pyridine;
6-(2-naphthyl)-2-(5-nitro-2-thienyl)-3-piperidin-4-yl-3H-imidazo[4,5-b]pyridine;
6-(2-naphthyl)-2-(1-oxidopyridin-4-yl)-3-piperidin-4-yl-3H-imidazo[4,5-b]pyridine;
7-(2-naphthyl)-3-piperidin-4-yl-1,3-dihydro-2H-imidazo[4,5-b]pyridine-2-thione;
tert-butyl (3S)-3-[7-(2-naphthyl)-3H-imidazo[4,5-b]pyridin-3-yl]piperidine-1-carboxylate;
tert-butyl 4-{[7-(2-naphthyl)-3H-imidazo[4,5-b]pyridin-3-yl]methyl}piperidine-1-carboxylate;
tert-butyl (3S)-3-[2-methyl-7-(2-naphthyl)-3H-imidazo[4,5-b]pyridin-3-yl]pyrrolidine-1-carboxylate;
2,2,6,6-tetramethyl-4-[7-(2-naphthyl)-3H-imidazo[4,5-b]pyridin-3-yl]piperidine-1-carbaldehyde;
2-methoxy-4-[6-(2-naphthyl)-3-piperidin-4-yl-3H-imidazo[4,5-b]pyridin-2-yl]phenol;
2-(2-bromophenyl)-6-(2-naphthyl)-3-piperidin-4-yl-3H-imidazo[4,5-b]pyridine;
2-(4-bromophenyl)-6-(2-naphthyl)-3-piperidin-4-yl-3H-imidazo[4,5-b]pyridine;
6-(2-naphthyl)-2-(3-phenoxyphenyl)-3-piperidin-4-yl-3H-imidazo[4,5-b]pyridine;
2-(3-fluorophenyl)-6-(2-naphthyl)-3-piperidin-4-yl-3H-imidazo[4,5-b]pyridine;
2-(1-methyl-1H-imidazol-2-yl)-6-(2-naphthyl)-3-piperidin-4-yl-3H-imidazo[4,5-b]pyridine;
2-(1-methyl-1H-pyrrol-2-yl)-6-(2-naphthyl)-3-piperidin-4-yl-3H-imidazo[4,5-b]pyridine;
2-(2-chloro-4-methyl-1-phenyl-1H-pyrrol-3-yl)-6-(2-naphthyl)-3-piperidin-4-yl-3H-imidazo[4,5-b]pyridine;
3-[6-(2-naphthyl)-3-piperidin-4-yl-3H-imidazo[4,5-b]pyridin-2-yl]quinoline;
2-(3-ethylphenyl)-6-(2-naphthyl)-3-piperidin-4-yl-3H-imidazo[4,5-b]pyridine;
or a tautomer thereof or pharmaceutically acceptable salt thereof.

21. The compound of claim 1, wherein the compound is 7-(2-naphthyl)-3-piperidin-4-yl-3H-imidazo[4,5-b]pyridine, or a pharmaceutically acceptable salt thereof.

22. A pharmaceutical composition comprising a compound of claim 1 or a tautomer thereof or pharmaceutically acceptable salt thereof and a pharmaceutically acceptable carrier.

23. A method of treating a canonical Wnt-β-catenin cellular messaging system related disorder, comprising administering to a mammal in need thereof a compound of claim 1 or a tautomer thereof or pharmaceutically acceptable salt thereof in an amount effect to treat a canonical Wnt-β-catenin cellular messaging system related disorder.

24. The method of claim 23, wherein the canonical Wnt-β-catenin cellular messaging system related disorder is selected from the group consisting of bone disorders, cancer, and Alzheimer's disease.

25. The method of claim 23, wherein the canonical Wnt-β-catenin cellular messaging system related disorder is cancer.

26. The method of claim 25, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

27. The method of claim 23, wherein the canonical Wnt-β-catenin cellular messaging system related disorder is Alzheimer's disease.

28. The method of claim 23, wherein the canonical Wnt-β-catenin cellular messaging system related disorder is a bone disorder.

29. The method of claim 28, wherein the bone disorder is selected from the group consisting of osteoarthritis, osteolysis from multiple myeloma, osteoporosis, and rheumatoid arthritis.

30. A process for preparing a compound of Formula (A₂): 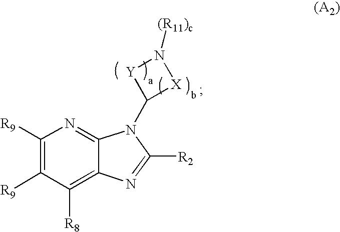
wherein R₂, R₈, R₉, R₁₁, X, Y, a, b, c and d are defined as in claim 1;
comprising deprotection of a compound of Formula VIII 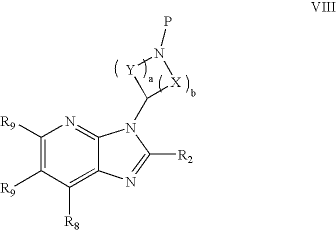
wherein P is an amino protecting group.

31. The process of claim 30, wherein, wherein P is a C₁-C₆ alkoxylcarbonyl.

32. The process of claim 30, wherein, wherein P is tert-butoxycarbonyl.

33. The process of claim 30, wherein the deprotection comprises treating the compound of Formula VIII with trifluoroacetic acid in presence of dichloromethane.

34. The process of claim 30, further comprising cyclization of a compound of Formula VII: 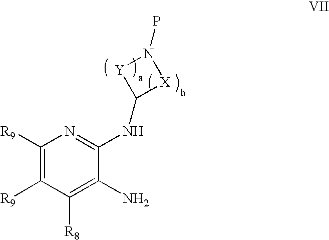
in presence of an R₂CHO or R₂COOH to provide Formula VIII; wherein P, X, Y, a, b, R₈, and R₉ are defined as in claim 30.

35. The process of claim 34, further comprising reducing a compound of Formula VI: 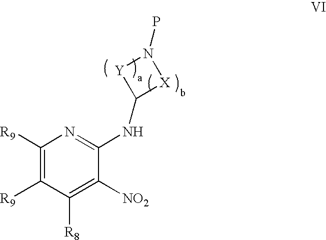
to provide the compound of Formula VII using hydrogen and palladium on carbon in presence of alcohol; wherein P, X, Y, a, b, R₈, and R₉ are defined as in claim 30.

36. The process of claim 35, further comprising substitution of G in Formula V: 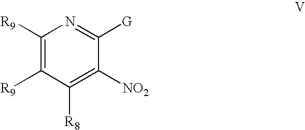
with a compound of Formula IV 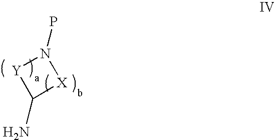
to provide the compound of Formula VI; wherein G is halogen; P, X, Y, a, b, R₈ and R₉ are defined as in claim 30.

37. The process of claim of 36, wherein the compound of Formula IV is

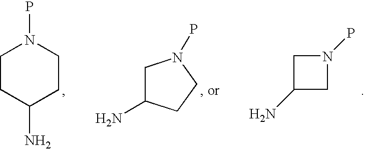

38. A compound of Formula (A₀): 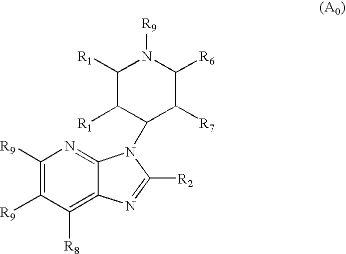
or tautomers thereof or pharmaceutically acceptable salts thereof, wherein
each R₁ is independently selected from H, C₁-C₆ alkyl, C₂-C₆ alkenyl, C₂-C₆ alkynyl, ═O, C₁-C₆ carbonyl, cycloalkyl, fluorinatedalkyl, CF₃, CN, NO₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, or heterocyclyl, wherein C₁-C₆ alkyl, C₂-C₆ alkenyl, C₂-C₆ alkynyl, C₁-C₆ alkoxy, aryl, heteroaryl, or heterocyclyl, are optionally substituted with one or more substituents selected from H, C₁-C₆ alkyl, C₂-C₆ alkenyl, C₂-C₆ alkynyl, ═O, C₁-C₆ carbonyl, cycloalkyl, fluorinatedalkyl, CF₃, CN, NO₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, or heterocyclyl, wherein C₁-C₆ alkyl, C₂-C₆ alkenyl, C₂-C₆ alkynyl, C₁-C₆ alkoxy, aryl, heteroaryl, and heterocyclyl;
R₂ is selected from H, C₁-C₆ alkyl, C₂-C₆ alkenyl, C₂-C₆ alkynyl, C₁-C₆ carbonyl, cycloalkyl, fluorinatedalkyl, CF₃, CN, NO₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, heterocyclyl, or fused aryl-heterocyclyl, wherein C₁-C₆ alkyl, C₂-C₆ alkenyl, C₂-C₆ alkynyl, C₁-C₆ alkoxy, aryl, heteroaryl, heterocyclyl, or fused aryl-heterocyclyl are optionally substituted with one or more R₁ groups;
R₃ is H, CF₃, C₁-C₆ alkyl, C₃-C₇ cycloalkyl, heterocyclyl, heterocyclyl alkyl, aryl, arylalkyl, heteroaryl, whereas all except H and CF₃ are optionally substituted with zero, one or more R₁ groups;
R₄ and R₅ are each independently H, aryl, heteroaryl, C₁-C₈ alkyl, cycloalkyl, heterocyclyl, arylalkyl, heteroarylalkyl, cycloalkyl-alkyl, heterocyclyl-alkyl, heterocyclyl-alkyl-aryl, heterocyclyl-alkoxyaryl, heterocyclyl-alkyl-heteroaryl, heterocyclyl-alkoxy-heteroaryl, alkoxyaryl, alkylamine-aryl, heterocyclyl-alkylamine-aryl, heterocyclyl-alkylamine-heteroaryl, whereas all except H are optionally substituted with zero, one or more R₁ groups; or R₄ and R₅ when taken together with the nitrogen to which they are attached form a 4 to 8 membered ring with 0, 1, or 2 additional heteroatoms optionally substituted with one or more R₁ groups;
R₆ and R₇ are each independently H, NH₂, CN, NO₂, C₁-C₆ alkyl, C₂-C₆ alkenyl, C₂-C₆ alkynyl, COOH, COOR₃, halogen, C₁-C₆ alkoxy, cycloalkyl, CF₃, S(O)ₙR₃ or OR₃; or R₆ and R₇ when taken together with the ring to which they are attached form C₅-C₇ cycloalkyl or C₆ aryl, optionally substituted with one or more R₁ groups;
R₈ is naphthlyl optionally substituted with one or more R₁ groups;
each R₉ is independently selected from H, C₁-C₆ alkyl, C₂-C₆ alkenyl, C₂-C₆ alkynyl, C₁-C₆ carbonyl, cycloalkyl, fluorinatedalkyl, CF₃, CN, NO₂, NH₂, halogen, COOR₃, C₁-C₆ alkoxy, OR₃, S(O)ₙR₃, NHC(O)C₁-C₆ alkyl, N(R₄)(R₅), SO₂N(R₄)(R₅), aryl, heteroaryl, heterocyclyl, wherein aryl, heteroaryl, heterocyclyl, C₁-C₆ alkyl, C₂-C₆ alkenyl, C₂-C₆ alkynyl, C₁-C₆ carbonyl, fluorinatedalkyl, or cycloalkyl are optionally substituted with one or more R₁ groups; and
n is 0, 1, or 2.
